SEQUENCE LISTING (104 sequences)

SEQ ID NO: 1 - protein, 782 aa, Homo sapiens
MSINLTVDIYIYLLSNARSVCGKQRSKQLYFLFSPKHYWRISHISLQRGFHTNIIRCKWTKSEAHSCSKHCYSPSNHGLHIGILKLSTSAPKGLTKVNICMSRIKSTLNSVSKAVFGNQNEMISRLAQFKPSSQILRKVSDSGWLKQKNIKQAIKSLKKYSDKSAEKSPFPEEKSHIIDKEEDIGKRSLFHYTSSITTKFGDSFYFLSNHINSYFKRKEKMSQQKENEHFRDKSELEDKKVEEGKLRSPDPGILAYKPGSESVHTVDKPTSPSAIPDVLQVSTKQSIANFLSRPTEGVQALVGGYIGGLVPKLKYDSKSQSEEQEEPAKTDQAVSKDRNAEEKKRLSLQREKIIARVSIDNRTRALVQALRRTTDPKLCITRVEELTFHLLEFPEGKGVAVKERIIPYLLRLRQIKDETLQAAVREILALIGYVDPVKGRGIRILSIDGGGTRGVVALQTLRKLVELTQKPVHQLFDYICGVSTGAILAFMLGLFHMPLDECEELYRKLGSDVFSQNVIVGTVKMSWSHAFYDSQTWENILKDRMGSALMIETARNPTCPKVAAVSTIVNRGITPKAFVFRNYGHFPGINSHYLGGCQYKMWQAIRASSAAPGYFAEYALGNDLHQDGGLLLNNPSALAMHECKCLWPDVPLECIVSLGTGRYESDVRNTVTYTSLKTKLSNVINSATDTEEVHIMLDGLLPPDTYFRFNPVMCENIPLDESRNEKLDQLQLEGLKYIERNEQKMKKVAKILSQEKTTLQKINDWIKLKTDMYEGLPFFSKL

SEQ ID NO: 2 - protein, 60 aa, Homo sapiens
MQAGWSGRHCRERASRRCSPSDCGLHPDCLLLQGLHDYLKFNNNARSVCGKQRSKQLYFL

SEQ ID NO: 3 - DNA, 320 bp, Homo sapiens
tggaagctcagctgatgcaggccggttggagtggacgtcattgccgggaacgagcgagtcgccgctgcagccctagtgactgcggcctgcatcccgattgtcttctcctccaaggtctacatgattacctgaagtttaataagtaagaccatgaattatggcatttcttaaatgaagcgttcaagaagtgagagaatgtcatagaaaataaatgatttttaagttatgtctattaatctgactgtagatatatatatttacctccttagtaatgcaagaagtgtttgtgggaagcagagaagcaagcaactgtatttctt

SEQ ID NO: 4 - DNA, 274 bp, Homo sapiens
  modified_base (121): a, t, c, g, unknown or other
tggaagctcagctgatgcaggccggttggagtggacttcattgccgggaacgagcgagtcgccgctgcagccctagtgactgcggcctgcatcccggtaagaccatgaattatggcatttnttaaatgaagcgttcaagaagtgagagaatgtcatanaaaataaatgatttttaagttatgtctattaatctgactgtagatatatatatttacctccttagtaatgcaagaagtgtttgtgggaagcagagaagcaagcaactgtatttctt

SEQ ID NO: 5 - DNA, 193 bp, Homo sapiens
  CDS (15)..(193)
tggaagctcagctgatgcaggccggttggagtggacgtcattgccgggaacgagcgagtcgccgctgcagccctagtgactgcggcctgcatcccgattgtcttctcctccaaggtctacatgattacctgaagtttaataataatgcaagaagtgtttgtgggaagcagagaagcaagcaactgtatttctt

SEQ ID NO: 6 - DNA, 3420 bp, Homo sapiens
  CDS (226)..(2571)
tggaagctcagctgatgcaggccggttggagtggacgtcattgccgggaacgagcgagtcgccgctgcagccctagtgactgcggcctgcatcccgattgtcttctcctccaaggtctacatgattacctgaagtttaataagtaagaccatgaattatggcatttcttaaatgaagcgttcaagaagtgagagaatgtcatagaaaataaatgatttttaagttatgtctattaatctgactgtagatatatatatttacctccttagtaatgcaagaagtgtttgtgggaagcagagaagcaagcaactgtatttcttgttctcacctaagcattactggaggataagccacatcagtctacaaagaggttttcatacaaacataataagatgtaaatggaccaaaagtgaagcacattcttgcagtaagcactgttactctccaagcaaccatggtttacatattgggattttgaaacttagcacttctgctcccaagggacttacaaaagtgaacatttgtatgtcccgtattaaaagtactttgaactctgtttcaaaggctgtttttggcaatcaaaatgaaatgatttcacgtttagctcaatttaagccaagttcccaaattttaagaaaagtatcggatagtggctggttaaaacagaaaaacatcaaacaagccatcaaatctctgaaaaaatatagtgacaaatcagcagaaaagagtccttttccagaagagaaaagtcacattatagacaaagaagaagatataggtaaacgcagtctttttcattacacaagttctataaccacaaaatttggagactcattctactttttatcaaatcatattaattcatatttcaaacgtaaggaaaaaatgtctcaacaaaaggaaaatgaacatttccgggacaaatcagaacttgaagataaaaaggtagaagaggggaaattaagatctccagatcctggcatcctggcttataagccaggctcagaatctgtacatacggtggacaagcctacaagtccttctgcgatacctgatgttcttcaagtttcaactaaacaaagtattgctaactttctttctcgtcccacggaaggtgtacaagctttagtaggtggttatattggtggacttgtccccaaattaaagtatgattcaaagagtcagtcagaagaacaggaagagcctgctaaaactgatcaggctgtcagcaaagacagaaatgcagaggagaaaaagcgtttatctcttcagcgagaaaagattatcgcaagggtgagtattgataacaggacccgggcattagttcaggcattaagaagaacaactgacccaaagctctgcattactagggttgaagaactgacttttcatcttctagaatttcctgaaggaaaaggagtggctgtcaaggaaagaattattccatatttattacgactgagacaaattaaggatgaaactcttcaggctgcagttagagaaattttggccctaattggctatgtggatccagtgaaagggagaggaatccgaattctctcaattgatggtggaggaacaaggggcgtggttgctctccagaccctacgaaaattagttgaacttactcagaagccagttcatcagctctttgattacatttgtggtgtaagcacaggtgccatattagctttcatgttggggttgtttcatatgcccttggatgaatgtgaggaactttatcgaaaattaggatcagatgtattttcacaaaatgtcattgttggaacagtaaaaatgagttggagccatgcattttatgacagtcaaacatgggaaaacattcttaaggataggatgggatctgcactgatgattgaaacagcaagaaaccccacatgtcctaaggtagctgctgtaagtaccatagtaaatagagggataacacccaaagcttttgtgttcagaaactatggtcattttcctggaatcaactctcattatttgggaggctgtcagtataaaatgtggcaggccattagagcctcatctgctgctccaggctactttgcagaatatgcattgggaaatgatcttcatcaagatggaggtttgcttctgaataacccttcggcattagctatgcatgagtgtaaatgtctttggccagatgtgccgttagagtgcatagtatccctgggcactggacgttatgagagtgatgtgagaaacacggtaacatacacaagcttgaaaactaaactttctaatgttatcaacagtgctacagatacagaagaagtccatataatgcttgatggcctgttacctcctgacacctattttagattcaatcctgtaatgtgtgaaaacatacctctagatgaaagtcgaaatgaaaagctggatcagctgcagttggaagggttgaaatacatagaaagaaatgaacaaaaaatgaaaaaagttgcaaaaatattaagtcaagaaaaaacaactctgcagaaaattaatgattggataaaattaaaaactgatatgtatgaaggacttccattcttttcaaaattgtgatgagtatatgcttatgttctcataaatgaaggtctgtttagaagatcaaccacattcaataaggaattgtggggttcgacatgagttaactttgaaatacgtatgaattctggagaatcctgaaaaagacggtgcttcaaccagcttgcatagcacagagaatattcttggttacagaattcatatgggaactaggcttttaagatgttaataattagctaagctttagtaacccttactgtgctagtagattttagtagatattggtgttatattgtttgatgtttgaaaatatattaatatatgtgccgaacaagaaaccgaaagctatattgtactgtgtatttttactttagtcctcataatcatgttgaatttatgtgatcattgattttatttcatatggaaaagctaatttcttcttaaatttacattacctaatattctcactagctatgttctccaatccacactgccttttattgtaatatcatctaaatagatgcagaaaaatggaattttctctattaaagtattttacatttgacataaaaaagaaccagatacagttttctattcagatatgtttattttaacattgtttggttaaaaaaggtgaagttccagtcaaccactttttacccctgaaatttcaagataatgctatattaacttttccagatctaacactagcttattcttccctgttataaaatggtttgaacttactgaggagatattcctatcattaacaaaaataaactatttaaataatctgttgttaaaaggctaatgtcattttaaaattaatttttgttcataatgtagctcccctttagcctttga

SEQ ID NO: 7 - DNA, 320 bp, Homo sapiens
aagaaatacagttgcttgcttctctgcttcccacaaacacttcttgcattactaaggaggtaaatatatatatctacagtcagattaatagacataacttaaaaatcatttattttctatgacattctctcacttcttgaacgcttcatttaagaaatgccataattcatggtcttacttattaaacttcaggtaatcatgtagaccttggaggagaagacaatcgggatgcaggccgcagtcactagggctgcagcggcgactcgctcgttcccggcaatgacgtccactccaaccggcctgcatcagctgagcttcca

SEQ ID NO: 8 - DNA, 274 bp, Homo sapiens
  modified_base (117): a, t, c, g, unknown or other
aagaaatacagttgcttgcttctctgcttcccacaaacacttcttgcattactaaggaggtaaatatatatatctacagtcagattaatagacataacttaaaaatcatttattttntatgacattctctcacttcttgaacgcttcatttaanaaatgccataattcatggtcttaccgggatgcaggccgcagtcactagggctgcagcggcgactcgctcgttcccggcaatgaagtccactccaaccggcctgcatcagctgagcttcca

SEQ ID NO: 9 - DNA, 193 bp, Homo sapiens
aagaaatacagttgcttgcttctctgcttcccacaaacacttcttgcattattattaaacttcaggtaatcatgtagaccttggaggagaagacaatcgggatgcaggccgcagtcactagggctgcagcggcgactcgctcgttcccggcaatgacgtccactccaaccggcctgcatcagctgagcttcca

SEQ ID NO: 10 - DNA, 92 bp, Artificial Sequence
  Description of Artificial Sequence  Synthetic oligonucleotide
atgatttcacgtttagctcaatttaagccaagttcccaaattttaagaaaagtatcggatagtggctggttaaaacagaaaaacatcaaaca

SEQ ID NO: 11 - DNA, 35 bp, Artificial Sequence
  Description of Artificial Sequence Primer
ttttgtcgacatgtctattaatctgactgtagata

SEQ ID NO: 12 - DNA, 38 bp, Artificial Sequence
  Description of Artificial Sequence Primer
gcatagcatgctcacaattttgaaaagaatgcaagtcc

SEQ ID NO: 13 - DNA, 2349 bp, Homo sapiens
  CDS (1)..(2346)
atgtctattaatctgactgtagatatatatatttacctccttagtaatgcaagaagtgtttgtgggaagcagagaagcaagcaactgtatttcttgttctcacctaagcattactggaggataagccacatcagtctacaaagaggttttcatacaaacataataagatgtaaatggaccaaaagtgaagcacattcttgcagtaagcactgttactctccaagcaaccatggtttacatattgggattttgaaacttagcacttctgctcccaagggacttacaaaagtgaacatttgtatgtcccgtattaaaagtactttgaactctgtttcaaaggctgtttttggcaatcaaaatgaaatgatttcacgtttagctcaatttaagccaagttcccaaattttaagaaaagtatcggatagtggctggttaaaacagaaaaacatcaaacaagccatcaaatctctgaaaaaatatagtgacaaatcagcagaaaagagtccttttccagaagagaaaagtcacattatagacaaagaagaagatataggtaaacgcagtctttttcattacacaagttctataaccacaaaatttggagactcattctactttttatcaaatcatattaattcatatttcaaacgtaaggaaaaaatgtctcaacaaaaggaaaatgaacatttccgggacaaatcagaacttgaagataaaaaggtagaagaggggaaattaagatctccagatcctggcatcctggcttataagccaggctcagaatctgtacatacggtggacaagcctacaagtccttctgcgatacctgatgttcttcaagtttcaactaaacaaagtattgctaactttctttctcgtcccacggaaggtgtacaagctttagtaggtggttatattggtggacttgtccccaaattaaagtatgattcaaagagtcagtcagaagaacaggaagagcctgctaaaactgatcaggctgtcagcaaagacagaaatgcagaggagaaaaagcgtttatctcttcagcgagaaaagattatcgcaagggtgagtattgataacaggacccgggcattagttcaggcattaagaagaacaactgacccaaagctctgcattactagggttgaagaactgacttttcatcttctagaatttcctgaaggaaaaggagtggctgtcaaggaaagaattattccatatttattacgactgagacaaattaaggatgaaactcttcaggctgcagttagagaaattttggccctaattggctatgtggatccagtgaaagggagaggaatccgaattctctcaattgatggtggaggaacaaggggcgtggttgctctccagaccctacgaaaattagttgaacttactcagaagccagttcatcagctctttgattacatttgtggtgtaagcacaggtgccatattagctttcatgttggggttgtttcatatgcccttggatgaatgtgaggaactttatcgaaaattaggatcagatgtattttcacaaaatgtcattgttggaacagtaaaaatgagttggagccatgcattttatgacagtcaaacatgggaaaacattcttaaggataggatgggatctgcactgatgattgaaacagcaagaaaccccacatgtcctaaggtagctgctgtaagtaccatagtaaatagagggataacacccaaagcttttgtgttcagaaactatggtcattttcctggaatcaactctcattatttgggaggctgtcagtataaaatgtggcaggccattagagcctcatctgctgctccaggctactttgcagaatatgcattgggaaatgatcttcatcaagatggaggtttgcttctgaataacccttcggcattagctatgcatgagtgtaaatgtctttggccagatgtgccgttagagtgcatagtatccctgggcactggacgttatgagagtgatgtgagaaacacggtaacatacacaagcttgaaaactaaactttctaatgttatcaacagtgctacagatacagaagaagtccatataatgcttgatggcctgttacctcctgacacctattttagattcaatcctgtaatgtgtgaaaacatacctctagatgaaagtcgaaatgaaaagctggatcagctgcagttggaagggttgaaatacatagaaagaaatgaacaaaaaatgaaaaaagttgcaaaaatattaagtcaagaaaaaacaactctgcagaaaattaatgattggataaaattaaaaactgatatgtatgaaggacttccattcttttcaaaattgtga

SEQ ID NO: 14 - DNA, 27 bp, Artificial Sequence
  Description of Artificial Sequence Primer
tgaacgtcgacatgtcccgtattaaaa

SEQ ID NO: 15 - protein, 682 aa, Homo sapiens
MSRIKSTLNSVSKAVFGNQNEMISRLAQFKPSSQILRKVSDSGWLKQKNIKQAIKSLKKYSDKSAEKSPFPEEKSHIIDKEEDIGKRSLFHYTSSITTKFGDSFYFLSNHINSYFKRKEKMSQQKENEHFRDKSELEDKKVEEGKLRSPDPGILAYKPGSESVHTVDKPTSPSAIPDVLQVSTKQSIANFLSRPTEGVQALVGGYIGGLVPKLKYDSKSQSEEQEEPAKTDQAVSKDRNAEEKKRLSLQREKIIARVSIDNRTRALVQALRRTTDPKLCITRVEELTFHLLEFPEGKGVAVKERIIPYLLRLRQIKDETLQAAVREILALIGYVDPVKGRGIRILSIDGGGTRGVVALQTLRKLVELTQKPVHQLFDYICGVSTGAILAFMLGLFHMPLDECEELYRKLGSDVFSQNVIVGTVKMSWSHAFYDSQTWENILKDRMGSALMIETARNPTCPKVAAVSTIVNRGITPKAFVFRNYGHFPGINSHYLGGCQYKMWQAIRASSAAPGYFAEYALGNDLHQDGGLLLNNPSALAMHECKCLWPDVPLECIVSLGTGRYESDVRNTVTYTSLKTKLSNVINSATDTEEVHIMLDGLLPPDTYFRFNPVMCENIPLDESRNEKLDQLQLEGLKYIERNEQKMKKVAKILSQEKTTLQKINDWIKLKTDMYEGLPFFSKL

SEQ ID NO: 16 - DNA, 2049 bp, Homo sapiens
  CDS (1)..(2046)
atgtcccgtattaaaagtactttgaactctgtttcaaaggctgtttttggcaatcaaaatgaaatgatttcacgtttagctcaatttaagccaagttcccaaattttaagaaaagtatcggatagtggctggttaaaacagaaaaacatcaaacaagccatcaaatctctgaaaaaatatagtgacaaatcagcagaaaagagtccttttccagaagagaaaagtcacattatagacaaagaagaagatataggtaaacgcagtctttttcattacacaagttctataaccacaaaatttggagactcattctactttttatcaaatcatattaattcatatttcaaacgtaaggaaaaaatgtctcaacaaaaggaaaatgaacatttccgggacaaatcagaacttgaagataaaaaggtagaagaggggaaattaagatctccagatcctggcatcctggcttataagccaggctcagaatctgtacatacggtggacaagcctacaagtccttctgcgatacctgatgttcttcaagtttcaactaaacaaagtattgctaactttctttctcgtcccacggaaggtgtacaagctttagtaggtggttatattggtggacttgtccccaaattaaagtatgattcaaagagtcagtcagaagaacaggaagagcctgctaaaactgatcaggctgtcagcaaagacagaaatgcagaggagaaaaagcgtttatctcttcagcgagaaaagattatcgcaagggtgagtattgataacaggacccgggcattagttcaggcattaagaagaacaactgacccaaagctctgcattactagggttgaagaactgacttttcatcttctagaatttcctgaaggaaaaggagtggctgtcaaggaaagaattattccatatttattacgactgagacaaattaaggatgaaactcttcaggctgcagttagagaaattttggccctaattggctatgtggatccagtgaaagggagaggaatccgaattctctcaattgatggtggaggaacaaggggcgtggttgctctccagaccctacgaaaattagttgaacttactcagaagccagttcatcagctctttgattacatttgtggtgtaagcacaggtgccatattagctttcatgttggggttgtttcatatgcccttggatgaatgtgaggaactttatcgaaaattaggatcagatgtattttcacaaaatgtcattgttggaacagtaaaaatgagttggagccatgcattttatgacagtcaaacatgggaaaacattcttaaggataggatgggatctgcactgatgattgaaacagcaagaaaccccacatgtcctaaggtagctgctgtaagtaccatagtaaatagagggataacacccaaagcttttgtgttcagaaactatggtcattttcctggaatcaactctcattatttgggaggctgtcagtataaaatgtggcaggccattagagcctcatctgctgctccaggctactttgcagaatatgcattgggaaatgatcttcatcaagatggaggtttgcttctgaataacccttcggcattagctatgcatgagtgtaaatgtctttggccagatgtgccgttagagtgcatagtatccctgggcactggacgttatgagagtgatgtgagaaacacggtaacatacacaagcttgaaaactaaactttctaatgttatcaacagtgctacagatacagaagaagtccatataatgcttgatggcctgttacctcctgacacctattttagattcaatcctgtaatgtgtgaaaacatacctctagatgaaagtcgaaatgaaaagctggatcagctgcagttggaagggttgaaatacatagaaagaaatgaacaaaaaatgaaaaaagttgcaaaaatattaagtcaagaaaaaacaactctgcagaaaattaatgattggataaaattaaaaactgatatgtatgaaggacttccattcttttcaaaattgtga

SEQ ID NO: 17 - DNA, 27 bp, Artificial Sequence
  Description of Artificial Sequence Primer
tcaagtcgacatgatttcacgtttagc

SEQ ID NO: 18 - protein, 661 aa, Homo sapiens [sequence omitted]

SEQ ID NO: 19 - DNA, 1986 bp, Homo sapiens
  CDS (1)..(1983)
atgatttcacgtttagctcaatttaagccaagttcccaaattttaagaaaagtatcggatagtggctggttaaaacagaaaaacatcaaacaagccatcaaatctctgaaaaaatatagtgacaaatcagcagaaaagagtccttttccagaagagaaaagtcacattatagacaaagaagaagatataggtaaacgcagtctttttcattacacaagttctataaccacaaaatttggagactcattctactttttatcaaatcatattaattcatatttcaaacgtaaggaaaaaatgtctcaacaaaaggaaaatgaacatttccgggacaaatcagaacttgaagataaaaaggtagaagaggggaaattaagatctccagatcctggcatcctggcttataagccaggctcagaatctgtacatacggtggacaagcctacaagtccttctgcgatacctgatgttcttcaagtttcaactaaacaaagtattgctaactttctttctcgtcccacggaaggtgtacaagctttagtaggtggttatattggtggacttgtccccaaattaaagtatgattcaaagagtcagtcagaagaacaggaagagcctgctaaaactgatcaggctgtcagcaaagacagaaatgcagaggagaaaaagcgtttatctcttcagcgagaaaagattatcgcaagggtgagtattgataacaggacccgggcattagttcaggcattaagaagaacaactgacccaaagctctgcattactagggttgaagaactgacttttcatcttctagaatttcctgaaggaaaaggagtggctgtcaaggaaagaattattccatatttattacgactgagacaaattaaggatgaaactcttcaggctgcagttagagaaattttggccctaattggctatgtggatccagtgaaagggagaggaatccgaattctctcaattgatggtggaggaacaaggggcgtggttgctctccagaccctacgaaaattagttgaacttactcagaagccagttcatcagctctttgattacatttgtggtgtaagcacaggtgccatattagctttcatgttggggttgtttcatatgcccttggatgaatgtgaggaactttatcgaaaattaggatcagatgtattttcacaaaatgtcattgttggaacagtaaaaatgagttggagccatgcattttatgacagtcaaacatgggaaaacattcttaaggataggatgggatctgcactgatgattgaaacagcaagaaaccccacatgtcctaaggtagctgctgtaagtaccatagtaaatagagggataacacccaaagcttttgtgttcagaaactatggtcattttcctggaatcaactctcattatttgggaggctgtcagtataaaatgtggcaggccattagagcctcatctgctgctccaggctactttgcagaatatgcattgggaaatgatcttcatcaagatggaggtttgcttctgaataacccttcggcattagctatgcatgagtgtaaatgtctttggccagatgtgccgttagagtgcatagtatccctgggcactggacgttatgagagtgatgtgagaaacacggtaacatacacaagcttgaaaactaaactttctaatgttatcaacagtgctacagatacagaagaagtccatataatgcttgatggcctgttacctcctgacacctattttagattcaatcctgtaatgtgtgaaaacatacctctagatgaaagtcgaaatgaaaagctggatcagctgcagttggaagggttgaaatacatagaaagaaatgaacaaaaaatgaaaaaagttgcaaaaatattaagtcaagaaaaaacaactctgcagaaaattaatgattggataaaattaaaaactgatatgtatgaaggacttccattcttttcaaaattgtga

SEQ ID NO: 20 - DNA, 27 bp, Artificial Sequence
  Description of Artificial Sequence Primer
gtaagtcgacaatgtctcaacaaaagg

SEQ ID NO: 21 - protein, 562 aa, Homo sapiens [sequence omitted]

SEQ ID NO: 22 - DNA, 1689 bp, Homo sapiens
  CDS (1)..(1686)
atgtctcaacaaaaggaaaatgaacatttccgggacaaatcagaacttgaagataaaaaggtagaagaggggaaattaagatctccagatcctggcatcctggcttataagccaggctcagaatctgtacatacggtggacaagcctacaagtccttctgcgatacctgatgttcttcaagtttcaactaaacaaagtattgctaactttctttctcgtcccacggaaggtgtacaagctttagtaggtggttatattggtggacttgtccccaaattaaagtatgattcaaagagtcagtcagaagaacaggaagagcctgctaaaactgatcaggctgtcagcaaagacagaaatgcagaggagaaaaagcgtttatctcttcagcgagaaaagattatcgcaagggtgagtattgataacaggacccgggcattagttcaggcattaagaagaacaactgacccaaagctctgcattactagggttgaagaactgacttttcatcttctagaatttcctgaaggaaaaggagtggctgtcaaggaaagaattattccatatttattacgactgagacaaattaaggatgaaactcttcaggctgcagttagagaaattttggccctaattggctatgtggatccagtgaaagggagaggaatccgaattctctcaattgatggtggaggaacaaggggcgtggttgctctccagaccctacgaaaattagttgaacttactcagaagccagttcatcagctctttgattacatttgtggtgtaagcacaggtgccatattagctttcatgttggggttgtttcatatgcccttggatgaatgtgaggaactttatcgaaaattaggatcagatgtattttcacaaaatgtcattgttggaacagtaaaaatgagttggagccatgcattttatgacagtcaaacatgggaaaacattcttaaggataggatgggatctgcactgatgattgaaacagcaagaaaccccacatgtcctaaggtagctgctgtaagtaccatagtaaatagagggataacacccaaagcttttgtgttcagaaactatggtcattttcctggaatcaactctcattatttgggaggctgtcagtataaaatgtggcaggccattagagcctcatctgctgctccaggctactttgcagaatatgcattgggaaatgatcttcatcaagatggaggtttgcttctgaataacccttcggcattagctatgcatgagtgtaaatgtctttggccagatgtgccgttagagtgcatagtatccctgggcactggacgttatgagagtgatgtgagaaacacggtaacatacacaagcttgaaaactaaactttctaatgttatcaacagtgctacagatacagaagaagtccatataatgcttgatggcctgttacctcctgacacctattttagattcaatcctgtaatgtgtgaaaacatacctctagatgaaagtcgaaatgaaaagctggatcagctgcagttggaagggttgaaatacatagaaagaaatgaacaaaaaatgaaaaaagttgcaaaaatattaagtcaagaaaaaacaactctgcagaaaattaatgattggataaaattaaaaactgatatgtatgaaggacttccattcttttcaaaattgtga

SEQ ID NO: 23 - DNA, 38 bp, Artificial Sequence
  Description of Artificial Sequence Primer
gcatactcgagtcacaattttgaaaagaatggaagtcc

SEQ ID NO: 24 - DNA, 22 bp, Artificial Sequence
  Description of Artificial Sequence Primer
gtacatacggtggacaagccta

SEQ ID NO: 25 - DNA, 33 bp, Artificial Sequence
  Description of Artificial Sequence Primer
cattcctctccctttcactggatccacatagcc

SEQ ID NO: 26 - protein, 16 aa, Artificial Sequence
  Description of Artificial Sequence Synthetic peptide
CENIPLDESRNEKLDQ

SEQ ID NO: 27 - DNA, 38 bp, Artificial Sequence
  Description of Artificial Sequence Primer
gaaaacctctttgtagactgatgtggcttatcctccag

SEQ ID NO: 28 - protein, 5 aa, Homo sapiens
ASCSV

SEQ ID NO: 29 - DNA, 302 bp, Homo sapiens
tcagcagggtcagaacctataatttcattcggtatattctgtatgggtgaagatgtacagccagcaaaagctttttaattcgggaaaacacgattggacttgcactttcaaaagattaccgtggttgcacagaagagactgactgggtcagaggttagttacaggctggaaaaccagtttagatgaaactgaagagcaaggatgaaagcctgaactagagcagtggaaatgcgaatgtggagcagaggaacgattcaagaaattctgcggtaaaactcatcagacttcatgactgattaaag

SEQ ID NO: 30 - DNA, 118 bp, Homo sapiens
ttttgcctttctagagtgttatacagctggaatcatactggtaggtctatggtccgaatgtttgtgcctctcaaaattcatgtggaaatcataaccgctaaagtgatggtattaagag

SEQ ID NO: 31 - DNA, 92 bp, Artificial Sequence
  Description of Artificial Sequence Synthetic oligonucleotide
tgtttgatgtttttctgttttaaccagccactatccgatacttttcttaaaatttgggaacttggcttaaattgagctaaacgtgaaatcat

SEQ ID NO: 32 - DNA, 28 bp, Artificial Sequence
  Description of Artificial Sequence Synthetic oligonucleotide
tcgacctgatttcacgtttagctcaatt

SEQ ID NO: 33 - DNA, 28 bp, Artificial Sequence
  Description of Artificial Sequence Synthetic oligonucleotide
tcgactaagccaagttcccaaattttaa

SEQ ID NO: 34 - DNA, 28 bp, Artificial Sequence
  Description of Artificial Sequence Synthetic oligonucleotide
tcgacgaaaagtatcggatagtggctgg

SEQ ID NO: 35 - DNA, 28 bp, Artificial Sequence
  Description of Artificial Sequence Synthetic oligonucleotide
tcgacttaaaacagaaaaacatcaaaca

SEQ ID NO: 36 - DNA, 28 bp, Artificial Sequence
  Description of Artificial Sequence Synthetic oligonucleotide
ggactaaagtgcaaatcgagttaaccgg

SEQ ID NO: 37 - DNA, 28 bp, Artificial Sequence
  Description of Artificial Sequence Synthetic oligonucleotide
gattcggttcaagggtttaaaattccgg

SEQ ID NO: 38 - DNA, 28 bp, Artificial Sequence
  Description of Artificial Sequence Synthetic oligonucleotide
gcttttcatagcctatcaccgaccccgg

SEQ ID NO: 39 - DNA, 28 bp, Artificial Sequence
  Description of Artificial Sequence Synthetic oligonucleotide
gaattttgtctttttgtagtttgtccgg

SEQ ID NO: 40 - protein, 353 aa, Mus sp.
MSINLTLDIYIYFLNNARSLCGKQRSKQLHFVCSKQYWRMNHVNVHREFHTSKKSCKWNRSEAHCSKHWHSPSNHGLHFGIVRLSTSAPKGLTKVSIHMSRIKSTLNSVSKAIFGSQNEMVTRLAQFKPSSRILRKVSDKGWLKQKNVKQAVESLKNYSDKSAGKNSLAEQKSYFADKEEDSGKHSLFHYTYGITTRFGESFSVLANHINSYFKSKGKMSQTKEDKQLQDKPDLEERKSSSPGPDTVADRPDSESPLEVKDKLSSPTQMPEAHPVSAKQSIANFLSRPTEGVQALVGGYIGGLVPKLKSDPKSPPEEQEVSAKTEQAVBKDKKAEEKKRVLLQQEKIIARVSI

SEQ ID NO: 41 - protein, 353 aa, Rattus sp.
MSINLILDIYIYFLNNARSFCGKQRSKQLNFLCSKQYWRMNHVNVHREFHTSKKSCKWNRSEAHCSKHWHSSSNHGVHIGIVKLSTSAPKGLTKVSIHMSRIKSTLNSVSKAIFGSQNEMVSRLAQFKPSSRIFRKVSDRGWLKHKNVKQAIESLKNYSDKSAEKNSFAEQKSYFADKEEGSDKHSLYHYAYRITTRFGESFYFLANHINSYFKNKEKMSQTKEDRQLQDKPCLEESKSISPSPDILTDRPDSGPPLNVEDKLSSSTQLPEALPVSTKQSIANFLSRPTEGVQALVGGYIGGLVPKLKSDPKSQPEEEEEPSKTDEPICKDKKAEEKKRVLLQREKIIARVSI

SEQ ID NO: 42 - protein, 359 aa, Homo sapiens
MSINLTVDIYIYLLSNARSVCGKQRSKQLYFLFSPKHYWRISHISLQRGFHTNIIRCKWTKSEAHSCSKHCYSPSNHGLHIGILKLSTSAPKGLTKVNICMSRIKSTLNSVSKAVFGNQNEMISRLAQFKPSSQILRKVSDSGWLKQKNIKQAIKSLKKYSDKSAEKSPFPEEKSHIIDKEEDIGKRSLFHYTSSITTKFGDSFYFLSNHINSYFKRKEKMSQQKENEHFRDKSELEDKKVEEGKLRSPDPGILAYKPGSESVHTVDKPTSPSAIPDVLQVSTKQSIANFLSRPTEGVQALVGGYIGGLVPKLKYDSKSQSEEQEEPAKTDQAVSKDRNAEEKKRLSLQREKIIARVSI

SEQ ID NO: 43 - DNA, 19 bp, Homo sapiens
  CDS (1)..(12)
cagcgagaaaaggcaagtt

SEQ ID NO: 44 - protein, 4 aa, Homo sapiens
QREK

SEQ ID NO: 45 - DNA, 18 bp, Homo sapiens
  CDS (1)..(18)
attatcgcaagggtgagt

SEQ ID NO: 46 - protein, 6 aa, Homo sapiens
IIARVS

SEQ ID NO: 47 - DNA, 26 bp, Homo sapiens
  CDS (1)..(20)
gaaaaggcaagttgttcagtgtgctt

SEQ ID NO: 48 - protein, 7 aa, Homo sapiens
EKASCSV

SEQ ID NO: 49 - protein, 5 aa, Artificial Sequence
  Description of Artificial Sequence Illustrative conserved lipase motif
GXSGS

SEQ ID NO: 50 - protein, 5 aa, Artificial Sequence
  Description of Artificial Sequence Illustrative conserved motif
GXSTG

SEQ ID NO: 51 - protein, 4 aa, Homo sapiens
IIAR

SEQ ID NO: 52 - protein, 7 aa, Homo sapiens
ICGVSTG

SEQ ID NO: 53 - protein, 10 aa, Homo sapiens
LSIDGGGTRG

SEQ ID NO: 54 - protein, 6 aa, Artificial Sequence
  Description of Artificial Sequence 6-His tag
HHHHHH

SEQ ID NO: 55 - DNA, 28 bp, Artificial Sequence
  Description of Artificial Sequence Primer
aaaagtcgacatgaagccatcaaatctc

SEQ ID NO: 56 - DNA, 27 bp, Artificial Sequence
  Description of Artificial Sequence Primer
aagagtcgacatgaggtaaacgcagtc

SEQ ID NO: 57 - DNA, 315 bp, Homo sapiens
atgtctattaatctgactgtagatatatatatttacctccttagtaatgcaagaagtgtttgtgggaagcagagaagcaagcaactgtatttcttgttctcacctaagcattactggaggataagccacatcagtctacaaagaggttttcatacaaacataataagatgtaaatggaccaaaagtgaagcacattcttgcagtaagcactgttactctccaagcaaccatggtttacatattgggattttgaaacttagcacttctgctcccaagggacttacaaaagtgaacatttgtatgtcccgtattaaa

SEQ ID NO: 58 - protein, 19 aa, Homo sapiens
MISRLAQFKPSSQILRKVS

SEQ ID NO: 59 - DNA, 45 bp, Homo sapiens
tattaatctgactgtagatatatatatttacctccttagtaatgc

SEQ ID NO: 60 - DNA, 6 bp, Homo sapiens
caagtg

SEQ ID NO: 61 - DNA, 6 bp, Homo sapiens
caggtg

SEQ ID NO: 62 - DNA, 6 bp, Homo sapiens
caggtg

SEQ ID NO: 63 - DNA, 31 bp, Artificial Sequence
  Description of Artificial Sequence Primer
gttgaagcttgtgtctattaatctgactgta

SEQ ID NO: 64 - DNA, 31 bp, Artificial Sequence
  Description of Artificial Sequence Primer
tagaccatggtggcttatcctccagtaatgc

SEQ ID NO: 65 - DNA, 32 bp, Artificial Sequence
  Description of Artificial Sequence Primer
gtgtaagcttgaagcagagaagcaagcaactg

SEQ ID NO: 66 - DNA, 35 bp, Artificial Sequence
  Description of Artificial Sequence Primer
actgccatggtggccttcacttttggtccatttac

SEQ ID NO: 67 - DNA, 29 bp, Artificial Sequence
  Description of Artificial Sequence Primer
tggaaagcttgccacatcagtctacaaag

SEQ ID NO: 68 - DNA, 32 bp, Artificial Sequence
  Description of Artificial Sequence Primer
tgctccatggtggcatcccaatatgtaaacca

SEQ ID NO: 69 - DNA, 34 bp, Artificial Sequence
  Description of Artificial Sequence Primer
gaaccaagcttgaagcacattcttgcagtaagca

SEQ ID NO: 70 - DNA, 35 bp, Artificial Sequence
  Description of Artificial Sequence Primer
caaaacatgttggctacgggacatacaaatgttca

SEQ ID NO: 71 - DNA, 31 bp, Artificial Sequence
  Description of Artificial Sequence Primer
gttgaagctttttgaaacttagcacttctgc

SEQ ID NO: 72 - DNA, 37 bp, Artificial Sequence
  Description of Artificial Sequence Primer
attccatggtggctgaaatcatttcattttgattgcc

SEQ ID NO: 73 - DNA, 29 bp, Artificial Sequence
  Description of Artificial Sequence Primer
tcaaaagcttatgatttcacgtttagctc

SEQ ID NO: 74 - DNA, 32 bp, Artificial Sequence
  Description of Artificial Sequence Primer
ctttccatggtggctgtcactatattttttca

SEQ ID NO: 75 - DNA, 18 bp, Homo sapiens
gattaaaggtatggtggt

SEQ ID NO: 76 - DNA, 19 bp, Homo sapiens
acctccttagtaatgcaag

SEQ ID NO: 77 - DNA, 22 bp, Homo sapiens
gcgtcacttccgctgggggcgg

SEQ ID NO: 78 - DNA, 11 bp, Homo sapiens
gccagtgtttg

SEQ ID NO: 79 - DNA, 13 bp, Homo sapiens
ttttaagttatgt

SEQ ID NO: 80 - DNA, 13 bp, Homo sapiens
aacatttgtatgt

SEQ ID NO: 81 - DNA, 13 bp, Homo sapiens
caaaatgaaatga

SEQ ID NO: 82 - DNA, 13 bp, Homo sapiens
aaggaaaaaatgt

SEQ ID NO: 83 - DNA, 13 bp, Artificial Sequence
  Description of Artificial Sequence Consensus sequence
gccgccrccatgg

SEQ ID NO: 84 - DNA, 270 bp, Homo sapiens
attataatactgtgcagcaagggcaataagagaagtgagcacaggtggaaggaatgattcattctatgagtagtgaggtaagattttcctggctgaaggacaaacaaatctttaggaggacaaggtggaaggggagctaagccaacagcatgaccaaggcactaagtatgaaaaggaacaagagtatctggggaagtacaggtgtggctggaggatagagagtgagaggcaagtggtgaaagtaaaggctggaaggtcagcagggtcaga

SEQ ID NO: 85 - DNA, 11 bp, Homo sapiens
ggacaggtggg

SEQ ID NO: 86 - DNA, 11 bp, Homo sapiens
cacaggtggtg

SEQ ID NO: 87 - DNA, 12 bp, Homo sapiens
cgacaggtggtg

SEQ ID NO: 88 - DNA, 64 bp, Mus sp.
gcgtcacttccgctgggggcggggcgtagcggtgggtggtgctggtcacgccagtgtttggggt

SEQ ID NO: 89 - DNA, 63 bp, Rattus sp.
gcgtcacttccgctgggggcggggcgtagcggagggtgaggctgtagcgccagtgtttggggt

SEQ ID NO: 90 - DNA, 76 bp, Homo sapiens
gcgtcacttccgctgggggcggagcggggcggggctgagtgggtggcgacctagctgctgcgccagtgtttgtgtt

SEQ ID NO: 91 - DNA, 18 bp, Homo sapiens
gcatcccggtaagtagcc

SEQ ID NO: 92 - DNA, 19 bp, Homo sapiens
acctccttagtaatgcaag

SEQ ID NO: 93 - DNA, 22 bp, Artificial Sequence
  Description of Artificial Sequence Synthetic oligonucleotide
gtggccgggtggtccacctcgg

SEQ ID NO: 94 - DNA, 39 bp, Artificial Sequence
  Description of Artificial Sequence Synthetic oligonucleotide
ttgatagttatctattacagtcttcttagattgaaacaa

SEQ ID NO: 95 - protein, 5 aa, Homo sapiens
LRKVS

SEQ ID NO: 96 - protein, 6 aa, Homo sapiens
GGGTRG

SEQ ID NO: 97 - protein, 5 aa, Homo sapiens
GVSTG

SEQ ID NO: 98 - DNA, 11 bp, Homo sapiens
tctcacctaag

SEQ ID NO: 99 - protein, 14 aa, Homo sapiens
NHGLIGILKLSTSA

SEQ ID NO: 100 - protein, 6 aa, Homo sapiens
KVNICM

SEQ ID NO: 101 - protein, 6 aa, Homo sapiens
NQNEMI

SEQ ID NO: 102 - protein, 4 aa, Homo sapiens
ISRL

SEQ ID NO: 103 - protein, 4 aa, Homo sapiens
IGKR

SEQ ID NO: 104 - DNA, 45 bp, Artificial Sequence; identical to SEQ ID NO: 59
  Description of Artificial Sequence Synthetic oligonucleotide

(19) United States
(12) Patent Application Publication
(10) Pub. No.: US 2005/0003388 A1
(43) Pub. Date: Jan. 6, 2005

(54) Calcium independent phospholipase A2upsilon polynucleotides and polypeptides and methods therefor

(76) Inventors: Richard W. Gross, Chesterfield, MO (US); David Mancuso, St. Louis, MO (US)

Correspondence Address: Patrick W. Rasche;Armstrong Teasdale LLP, One Metropolitan Square, Suite 2600, St. Louis, MO, 63102, US

(21) Appl. No.: 10/786,505

(22) Filed: Feb. 25, 2004

Related U.S. Application Data
(63) Continuation-in-part of application No. 09/618,623, filed on Jul. 18, 2000, now abandoned.

(51) Int. Cl. C12Q 1/68; C07H 21/04; C12N 9/20; A01H 1/00; C12N 15/82; C12N 5/04

(52) U.S. Cl. 435/6; 800/281; 435/69.1; 435/198; 435/419; 435/468; 536/23.2

(57) ABSTRACT

A novel human phospholipase, iPLA₂γ (iPLA₂γ), is described and characterized. The complete genomic organization, complete nucleic acid sequence, and amino acid sequence have been identified. The sn-2-lipase activity of iPLA₂γ is also described. In addition, utilitarian methods for detecting and monitoring patient levels of iPLA₂γ are described, as well as methods for treating disease conditions by modulating the activity of iPLA₂γ.

### CROSS-REFERENCE TO RELATED APPLICATIONS

This Application is a continuation in part of pending U.S. application Ser. No. 09/168,623 filed Jul. 18, 2000, which is incorporated in its entirety by reference.

### REFERENCE TO GOVERNMENT GRANT

This invention was made with U.S. government support under National Institute of Health Grant Numbers 1 PO1 HL 57278-02, 2 RO2 HL 41250-08A1, and 5RO1 AA11094. The government has certain rights in this invention.

### STATEMENT REGARDING SEQUENCE LISTING

Applicants assert that the attached paper copy of the Sequence Listing for this utility application, a Continuation-in-Part (C.I.P.) to U.S. patent application Ser. No. 09/618,623, filed Jul. 18, 2000 pending, is identical to the Sequence Listing in computer readable form found on the accompanying computer disk, as required by 37 CFR 1.821 (c) and is hereby incorporated by reference in its entirety.

### FIELD OF THE INVENTION

This invention relates to phospholipases and, more particularly, to novel calcium-independent phospholipase A₂γ polypeptides and to nucleic acids encoding these polypeptides, as well as to methods of making and using these nucleic acids and polypeptides.

### BACKGROUND OF THE INVENTION

The function of complex living biological organisms relies on the meticulous control of cellular activity, including close regulation of cell growth, proliferation and function. The family of enzymes known as the phospholipases A₂ has been implicated in the control of cellular activity by catalyzing the esterolytic cleavage of fatty acids from phospholipids, thereby regulating the release of lipid second messengers, cellular growth factors, and the properties of the cellular membrane (Samuelsson et al., Annu. Rev. Biochem. 47:997-1029, 1978; Moolenaar, W. H., Curr. Opin. Cell. Biol. 7:203-10, 1995). In particular, by controlling the production of second messengers such as arachidonic acid and its biologically active eicosanoid metabolites, phospholipases A₂ are involved in modulating such processes as cellular growth programs, inflammation, vascular tone and ion channel function. (Needleman et al., Annu. Rev. Biochem. 55:69-102, 1986).

However, phospholipases A₂ are a broad family of enzymes with varying kinetic and physical properties, and distinct functions. Early research focused on distinguishing broad classes of the enzymes within the larger family. Several classes were distinguished using in vitro activity assays, and are categorized based on the dependence of their enzymatic activity on the presence of calcium ion. (See e.g., Demel et al, Biochim. Biopliys. Acta 406:97-107,1975). Thus, one class, the secretory phospholipases A₂ are distinguished by an obligatory dependence on high (millimolar) concentrations of calcium ions, as well as low molecular weights (14-18 kDa) and relative heat stability. (Demel et al., supra; Tischfield, J. A., J. Biol. Chem. 272:17247-50, 1997). The activity of a second class, the calcium-facilitated phospholipases A₂ is facilitated by the presence of nanomolar concentrations of calcium ions, but the presence of the calcium ion is not obligatory. (Loeb et al., J. Biol. Chem. 261:10467-70, 1986; Kramer et al., Biochim. Biophys. Acta 878:394-403; Glover et al., J. Biol. Chem. 270:15359-67, 1986). A third class of enzymes is entirely calcium-independent in vitro studies, and is also distinguished by a finely tuned inhibition by (E)-6-(bromomethylene)-3-(1-napthalenyl)-2H-tetrahydropyran-2-one (BEL). (Wolf et al., J. Biol. Chem. 260:7295-303; Hirashima et al., J. Neurochem. 59:708-14; Lehman et al., J. Biol. Chem. 268:20713-16).

Application of molecular biological techniques has provided some insights into the structure and function of founding members in each class of phospholipases A₂ and has provided a further basis for distinguishing among the classes. (See, e.g. Demel et al, supra; Evenberg et al., J. Biol. Chem. 252: 1189-96, 1977; Tischfeld, J. A., J. Biol. Chem. 272: 17247-50, 1997). For example, members of the secretory phospholipases A₂ use a calcium ion to polarize the carbonyl for attack by a histidine-activated H₂O molecule, while the intracellular phospholipases use a nucleophilic serine. The calcium-facilitated phospholipases A₂ have a GXSGS consensus lipase motif (SEQ ID NO: 49), in contrast to the iPLA₂ group which has a GXSTG consensus motif (SEQ ID NO: 50). The calcium-independent phospholipases A₂ are also distinguished by a consensus sequence for nucleotide binding. (Andrews et al., Biochem. J. 252:199-206, 1988; Tang et al., J. Biol. Chem. 272:8567-75, 1998). These findings have clearly boosted progress toward identifying the polypeptides responsible for catalyzing the synthesis of the eicosanoid metabolites and toward understanding the regulatory mechanisms of phospholipases A₂ that are involved in normal and disease states.

The more recent developments of intense genome sequencing efforts have produced partial sequence data on the phospholipases and led to related structural insights. For example, two new calcium-facilitated phospholipases have recently been described based on data from protein and nucleotide databases. (Underwood et al., J. Biol. Chem. 273: 21926-32, 1998; Pickardetal., J Biol. Chem. 274: 8823-31, 1999). Further, during sequencing of the long arm of chromosome 7 in the Human Genome Sequencing Project, a predicted protein product of 40 kDa was identified. The polypeptide contained two 10 amino acid segments homologous to the lipase and nucleotide-binding consensus sequences described for the founding members of the iPLA₂ family. (Tang et al., supra).

Earlier work has suggested a connection between some types of phospholipases and certain disease conditions in animals. For example, intensive study of reperfusion injury in myocardial tissue has led to the hypothesis that pathology is ultimately generated because of membrane phospholipid breakdown attributable to activation of myocardial phospholipase A₂ activity. (See e.g. Van der Vusse et al., Hydrolysis of phospholipids and cellular integrity, In: H. M. Piper (ed.) Pathophysiology of Severe Ischemic Myocardial injury, Kluwer Academic Publishers, Dordrecht, The Netherlands, 1990, 167-93). Furthermore, calcium-dependent and calcium-independent phospholipase A₂ activities have also been found to be present in the human cerebral cortex. Some reports have suggested a possible link between the activity of both calcium-dependent calcium independent phospholipases A₂ and cortical degenerative diseases such as Alzheimer's disease. (For reviews, see e.g. Farooqui et al., Neurochem. Int. 30: 5 17-22, 1997; Farooqui et al., Brain Res. Bull. 49: 139-53, 1999).

Certain inhibitors of phospholipases A₂ have been identified as possible therapeutic candidates for treating PLA₂-mediated diseases. For example, fatty acid trifluoromethyl ketones, bromoenol lactone, methyl arachidonyl fluorophosphonate, bezenesulfonamides and other specific inhibitors of phospholipases A₂ have been shown to decrease PLA₂ activity and all have been considered for treating inflammatory diseases thought to be mediated by PLA₂. (See e.g. Farooqui et al, 1999, supra). Nevertheless, as noted above, the phospholipases A₂, as well as the iPLA₂ subfamily itself, are a heterogeneous group of enzymes, with differing molecular weights, substrates, and responses to inhibitors. Because of this, the development of agents for treating diseases mediated by these compounds is ideally based upon determining and characterizing the structure and functional characteristics of the particular iPLA₂ involved in the disease process. Thus, it is important to identify and characterize the phospholipases A₂ family members of interest in order to identify and develop new drugs with specific affinity and/or activity for those phospholipases A₂.

### BRIEF SUMMARY OF THE INVENTION

Accordingly, the inventors have discovered a novel phospholipase A₂, referred to herein as calcium-independent phospholipase A₂γ (iPLA₂γ), and nucleic acid sequences encoding and expressing iPLA₂γ. The novel calcium-independent phospholipase A₂γ is involved with supplying fatty acids for β-oxidation or hydrolyzing lipids to provide signaling molecules which regulate energy storage and bioenergenetics and is believed to be involved in certain disease processes in animals. More specifically, the present invention is based on the collective work reported herein which identifies the complete genomic organization, nucleic acid sequence, and lipase activity of the novel calcium-independent membrane-associated phospholipase A₂.

In one embodiment, the invention is directed to an isolated nucleic acid molecule comprising a set of iPLA₂γ polynucleotides. In one aspect of this embodiment, the iPLA₂γ polynucleotides encode and express an iPLA₂γ polypeptide. The iPLA₂γ polypeptide catalyzes cleavage of fatty acids from the phospholipids by either sn-1 or sn-2 hydrolysis. Preferred sequences of the iPLA₂γ polypeptide are as set forth in SEQ ID NO: 1 and SEQ ID NO: 2. SEQ ID NO: 1 represents the amino acid sequences corresponding to alternative splice variants of iPLA₂γ, identified herein as gamma 1 and gamma 2, respectively. The two splice variants involve splice sites 5′ to the start codon so that both splice variants gamma 1 and 2 encode the same iPLA₂γ polypeptide SEQ ID NO: 1. However, splice variant gamma 3, SEQ ID NO:5 utilizes a start codon 5′ to that of splice variant gamma 1 and splice variant gamma 2 to encode a longer polypeptide SEQ ID NO:2.

SEQ ID NO:3 and SEQ ID NO:4 constitute the cDNA of the coding regions of splice variant gamma 1 and 2, respectively. SEQ ID NO:5 constitutes the cDNA of the coding region of splice variant gamma 3. SEQ ID NO: 6 is the nucleotide sequence for the full length iPLA₂γc DNA.

The present invention is also directed to iPLA₂γ polynucleotides and fragments thereof which specifically hybridize under stringent conditions to the complements of the coding regions of iPLA₂γ polynucleotides set forth in SEQ ID NO:3, SEQ ID NO:4, and SEQ ID NO:5, and their corresponding complements, SEQ ID NO:7, SEQ ID NO:8, and SEQ ID NO:9, respectively.

In another aspect of this embodiment, the present invention is directed to iPLA₂γ polynucleotides comprising polynucleotides having at least about 90% identity with SEQ ID NO:3, SEQ ID NO:4, SEQ ID NO:5 or SEQ ID NO:6, SEQ ID NO:7, SEQ ID NO:8 and SEQ ID NO:9; and, preferably, such polynucleotides comprising SEQ ID NO:3, SEQ ID NO:4, SEQ ID NO:5, or SEQ ID NO:6, or SEQ ID NO:7, SEQ ID NO:8, and SEQ ID NO:9.

In another aspect of this embodiment, the present invention is also directed to vectors and cells comprising a polynucleotide encoding an iPLA₂γ polypeptide. In addition, the present invention encompasses antisense compounds which specifically hybridize to SEQ ID NO: 3, SEQ ID NO:4, SEQ ID NO:5 or SEQ ID NO: 6.

The present invention is also directed to iPLA₂γ polynucleotides (SEQ ID NOS 13, 16, 19, and 22) which encode full-length or truncated polypeptides (SEQ ID NOS 1, 15, 18, 21) through use of alternative ATG start sites. Each of these constructs is identical but truncated compared to full-length iPLA₂γ (SEQ ID NO: 1) including sequence corresponding to the lipase active site encoding amino acids 481-485) of iPLA₂γ and thus each potentially retains catalytic activity. However, it is expected that modifications such as removal of an N-terminal membrane binding domain through use of a downstream ATG start site (or use of an alternative promoter through alternative splicing) will have profound effects on the functional properties of the polypeptide. These changes may increase or decrease enzymatic activity or specificity or change the intracellular site at which the polypeptide's activity is exerted. In fact, the major thrust of this patent is to describe the diversity of this molecule (in terms of splicing variants, use of alternative promoters, and use of downstream ATG start sites) with the implication being that the functional properties of this polypeptide are highly regulated by these diverse modifications.

It is possible that the full-length nucleotide sequence (SEQ ID NO: 13) encoding the 88 kDa polypeptide (SEQ ID NO: 1) may not represent the true endogenous form of iPLA₂γ having significant biologically functional activity. When constructs containing the full length 88 kDa form (SEQ ID NO: 13) are expressed in the baculovirus system and in transgenic mice, 74 and 63 kDa polypeptides are expressed. These results may indicate that the true endogenous activity derives from these truncated forms. It is also equally possible (and likely) that different forms of iPLA₂γ derived from differential splicing or use of alternative ATG start sites or other mechanisms) are expressed to different degrees under different biological conditions. For example, we show that canine iPLA₂γ is expressed both as different sized isoforms and at different levels in aorta vs. liver (FIG. 30). Likewise, functional regulation of iPLA₂γ in normal and pathological conditions may also be regulated at transcriptional and translational levels by generation of a alternatively spliced forms, alternative promoter usage, and use of alternative ATG start sites as well as by other mechanisms.

In a particular aspect of this embodiment, the present invention is directed to a fragment of an iPLA₂γ polynucleotide comprising an iPLA₂γ repressor binding site. Preferably, the polynucleotide comprises SEQ ID NO:10, 5′-ATGATTTCACGTTTAGCTCAATTTAAGCCAAGTTCCCAAATTTTAAGAAAAGTAT CGGATAGTGGCTGGTTAAAACAGAAAAACATCAAACA-3′ or its complement SEQ ID NO:31 or subparts thereof further comprising SEQ ID NO:32, 33, 34, 35 or their complementary oligonucleotide pairs including appropriate sticky ends for ligation (SEQ ID NOS 36, 37, 38, or 39).

In another embodiment, the present invention is directed to an isolated polypeptide comprising a phospholipase A₂γ. The isolated polypeptide catalyzes cleavage of fatty acids from the phospholipids. In one aspect of this embodiment, the polypeptide has at least about 90% identity with SEQ ID NO:1 or SEQ ID NO:2; and, more preferably, the polypeptide comprises SEQ ID NO:1 or SEQ ID NO:2.

In another aspect of this embodiment, the present invention comprises a conservatively substituted variant of SEQ ID NO:1 or SEQ ID NO:2. The present invention also encompasses antibodies capable of binding to a phospholipase A₂γ polypeptide.

Another embodiment of the present invention constitutes a method of treating inflammation in a patient. The method comprises treating the patient in need of such treatment to alter (decrease or increase) calcium-independent phospholipase A₂γ activity in the patient. It is contemplated that patients in need of such treatment include those patients suffering from one or more of Alzheimer's disease, myocardial ischemia and myocardial infarction. Preferably, one method comprises administering to the patient an effective amount of phospholipase A₂γ translational repressor molecule.

As used herein, the term “patient” includes humans and other living animals.

In another aspect of this invention, the method comprises administering to the patient an antisense sequence which specifically hybridizes to SEQ ID NO:3 or SEQ ID NO:4 or SEQ ID NO:5.

In another embodiment, the present invention is directed to a method of modulating fatty acid utilization in a living patient in need thereof. In this aspect, the method comprises increasing or decreasing iPLA₂γ activity in the patient. Patients in need of such treatment include those patients suffering from diabetes and/or obesity. In an aspect, this method comprises administering to the patient a substance in an effective amount which blocks translational repression of iPLA₂γ, which results in increasing iPLA₂γ activity in the patient. In another aspect of this invention, the method comprises administering to the patient an iPLA₂γ polypeptide or a polynucleotide encoding said iPLA₂γ polypeptide.

Another embodiment of the present invention is directed to a method for measuring activity of a phospholipase A₂γ polypeptide of cells in a biological sample. The method comprises introducing into the sample a phospholipid substrate for cleavage of fatty acids by the phospholipase A₂γ, wherein the phospholipase A₂γ cleaves fatty acid from either the sn-1 or sn-2-position of the phospholipid substrate. This method further comprises measuring cleavage of the phospholipid substrate and/or quantifying the release of fatty acid from the substrate.

The present invention is also directed to an assay method for identifying substances which modulate iPLA₂γ expression in a cell. The method comprises effectively contacting a candidate substance with cells comprising a promoter sequence operably linked to an iPLA₂γ repressor or enhancer binding site and a reporter gene and measuring expression of the reporter gene. Alternatively, the method comprises contacting a candidate substance with cells comprising an iPLA₂γ promoter sequence operably linked to a reporter gene and measuring expression of the reporter gene. By iPLA₂γ repressor or enhancer binding site it is meant that portion of the nucleotide sequence of an iPLA_{2γ} polynucleotide to which one or more endogenous substances within a cell binds to regulate transcription or translation of that iPLA₂γ polynucleotide.

In one embodiment, the iPLA₂γ repressor binding site comprises SEQ ID NO:10 or fragments SEQ ID NOS 32, 33, 34 and 35 or their complementary oligonucleotide pairs including appropriate sticky ends for ligation (SEQ ID NOS 36-39).

In another embodiment, the iPLA₂γ promoter comprises sequences (generally within 2 kb) upstream from iPLA₂γ exons 1 or 2. The reporter gene preferably encodes an enzyme capable of being detected by a suitable colorimetric, fluorometric or luminometric assay such as, for example, a reporter sequence encoding and expressing a luciferase. In a particularly preferred embodiment, the promoter sequence is a baculovirus promoter sequence and the cells are Sf9 cells.

The present invention also encompasses genetically engineered cells capable of identifying substances which modulate iPLA₂γ expression in a cell. Such cells comprise a promoter operably linked to an iPLA₂γ repressor binding site and a reporter gene, or alternatively an iPLA₂γ promoter linked to a reporter gene. Preferably, the iPLA_{2γ} repressor binding site comprises SEQ ID NO:10 while the iPLA₂γ promoter comprises a sequence within 2 kb upstream of exons 1 or 2. This reporter gene preferably encodes an enzyme capable of being detected by a suitable colorimetric, fluorometric or luminometric assay such as, for example, a reporter sequence encoding a luciferase. In an aspect, the promoter sequence is a baculovirus promoter sequence and the cells are Sf9 cells.

The present invention includes a method for identifying substances which modulate iPLA₂γ expression. In an aspect, the method comprises contacting a candidate substance with cells capable of expressing iPLA₂γ or a fragment thereof, and measuring the expression of iPLA₂γ or a fragment thereof by the cells. A level of expression greater or less than expression in an absence of the substance indicates and is determinant of activity in modulating iPLA₂γ expression.

Therefore, the present invention affords several advantages, including the provision of a novel phospholipase A₂γ, iPLA₂γ, which catalyzes the release of fatty acids from the sn-1 or sn-2-position of phospholipid substrate; the provision of methods for identifying a substance which represses or enhances and thus controls iPLA₂γ expression in a sample; the provision of methods for treating diseases involving inflammatory processes, inflammation or fatty acid utilization; and the provision of methods for determining the presence and level of activity or expression of iPLA₂γ in a sample.

In one embodiment, a transcriptional repressor region was defined including nucleotide residues 1-315 (SEQ ID NO: 57) of the 88 kDa coding sequence (SEQ ID NO: 13) utilizing gel shift analysis.

In another embodiment, a human iPLA₂γ N-terminal mitochondrial localization signal and cleavage site are defined comprising sequence MISRLAQFKPSSQILRKAVS (SEQ ID NO: 58). Cleavage occurs after the lysine (K) in iPLA_{2γ}.

In one embodiment, a transcription factor binding region is defined between nucleotide residues 6-50 encoding the 88 kDa protein and including the sequence 5′-TATTAATCTGACTGTAGATATATATATTTACCTCCTTAGTAATGC-3 (SEQ ID NO: 59) within the N-terminal coding region of iPLA₂γ by gel shift analysis.

In another embodiment, three MyoD transcription factor binding sites (E-boxes) defined by the consensus nucleotide sequence CANNTG are identified in promoter 1 (pre exon 1) sequence of the iPLA₂γ gene. The MyoD binding sites are located at nucleotide residues −22 through −27 corresponding to nucleotide sequence 5′-CAAGTG-3′ (SEQ ID NO: 60), −53 through −58 corresponding to nucleotide sequence 5′-CAGGTG-3′ (SEQ ID NO: 61), and −349 through −354 corresponding to nucleotide sequence 5′-CAGGTG-3′ (SEQ ID NO:62) upstream from start of exon 1(see SEQ ID NO: 29 in Table I).

In another embodiment, utilizing gel shift analysis, we provide evidence for an initiator (Inr) sequence with a consensus sequence of Py-Py-A-N-T/A-Py-Py at which nuclear protein constituents bind to 5′-GCG TCA CTT CCG CTG GGG GCG G-3′ (SEQ ID NO: 77) at nucleotide residues −54 through −75 upstream from the putative start of exon 2 (FIG. 39). The Inr as well another sequence 5′-GCCAGTGTTTG-3′ (SEQ ID NO 78) which is consistent with sequence for a CORE promoter element are highly conserved in comparisons of human, mouse, and rat sequence within this region of the iPLA₂γ gene (see FIG. 39).

### BRIEF DESCRIPTION OF THE DRAWINGS

FIG. 1 shows the iPLA₂γ cDNA sequence (SEQ ID NO:6) which contains the coding region for the 88 kDa iPLA₂γ polypeptide along with the corresponding amino acid sequence (SEQ ID NO:1).

FIG. 2 illustrates three alternative splice variants of the iPLA₂γ gene.

FIG. 3 illustrates the 5′ portion of three alternative splice variants, identified as 1 (SEQ ID NO:3), 2 (SEQ ID NO:4) and 3 (SEQ ID NO:5) along with amino terminal portion of the encoded polypeptides corresponding to splice variants 1 and 2 (SEQ ID NO:1) and splice variant 3 (SEQ ID NO:2).

FIG. 4 illustrates the full-length 88 kDa polypeptide (SEQ ID NO:1) and the cDNA encoding the polypeptide (SEQ ID NO:13) along with sense primer M444 (SEQ ID NO:11) and reverse primer M458 (SEQ ID NO:12) used in the PCR (polymerase chain reaction) amplification of the 88 kDa polypeptide.

FIG. 5 illustrates the 77 kDa truncated polypeptide starting at amino acid 101 and nucleotide 301 (SEQ ID NO:15) and the cDNA encoding the polypeptide (SEQ ID NO: 16) along with sense primer m534 (SEQ ID NO: 14) and reverse primer M458 (SEQ ID NO:12) used in the PCR amplification of the 74 kDa polypeptide.

FIG. 6 illustrates the 74 kDa truncated polypeptide starting at amino acid 122 and nucleotide 364 (SEQ ID NO:18) and the cDNA encoding the polypeptide (SEQ ID NO:19) along with sense primer m533 (SEQ ID NO:17) and reverse primer M458 (SEQ ID NO:12) used in the PCR amplification of the 74 kDa polypeptide.

FIG. 7 illustrates the 63 kDa truncated polypeptide starting at amino acid 221 and nucleotide 661 (SEQ ID NO:21) and the cDNA encoding the polypeptide (SEQ ID NO:22) along with sense primer m530 (SEQ ID NO:20) and reverse primer M458 (SEQ ID NO:12) used in the PCR amplification of the 74 kDa polypeptide.

FIG. 8 is a schematic illustration mapping expressed sequence tags (ESTs) overlapping the full-length sequence of iPLA₂γ.

FIG. 9 illustrates the analysis of PCR amplified human iPLA₂γ on agarose gel electrophoresis.

FIG. 10 shows a potential alternative exon 5 splice variant of human iPLA₂γ. In gene splicing, introns typically begin with the dinucleotide “gt” and end with “ag”. This gt/ag splice signal occurs with a frequency of 98.71% in gene splicing and can be used as a reliable tool for identifying the boundaries of exons in genes. The previously reported 3′ splice signal of iPLA₂γ exon 5 is illustrated in ‘A’. This splice site utilizes an unusual gc/ag splice signal which is utilized at a frequency of only 0.56% in splicing of genes. The use of the more common gt/ag splice signal will result in splicing shown in ‘B’. As illustrated, this splicing will not alter the reading frame of the encoded protein but will result in a modified portion of the iPLA₂γ sequence which contains the sequence “ASCSV” (SEQ ID NO: 28) in place of “ILIAR” (SEQ ID NO: 51) shown in ‘A’. Sequence encoding the altered sequence in ‘B’ has not yet been cloned from a cDNA library nor has sequence with identity to SEQ ID NO: 28 been reported in GenBank® (NIH genetic sequence database). However, because of the extensive alternative splicing that the iPLA₂γ gene undergoes (at least 10 splice variants), it is at least plausible that the more common gt/ag splicing may be utilized at the 3′ end of exon 5 resulting in a polypeptide containing SEQ ID NO: 28. Because of the proximity of the amino acids affected by this alternative splicing to the lipase consensus site (within 126 amino acids), the generation of a polypeptide with SEQ ID NO: 28 could potentially result in modulation of iPLA₂γ activity or functional properties.

FIG. 11 is a hydropathy plot analysis of the deduced amino acid sequence of human iPLA₂γ.

FIG. 12 is a Northern blot analysis of human iPLA₂γ mRNA obtained from multiple tissue samples.

FIG. 13 is a schematic illustration of the genomic organization of the human iPLA₂γ gene.

FIG. 14 shows the results of gel electrophoresis and autoradiographic visualization of the in vitro translation products of iPLA₂γ.

FIG. 15 shows the results of Western blot analysis of iPLA₂γ expression in Sf9 cells.

FIG. 16 is a graph of an initial rate analysis of the iPLA₂ activity of iPLA₂γ.

FIG. 17 is a substrate selectivity profile of the iPLA₂ activity of iPLA₂γ.

FIG. 18 is a graph of inhibition of iPLA₂ activity of iPLA₂γ by BEL.

FIG. 19 shows the results of Western analysis of expression of truncated iPLA₂γ constructs in Sf9 cells.

Western analysis is sometimes called Western blotting or immunoblotting and is a reliable method for checking any sample for the presence of a specific antigen. The detection is based on the molecular weight of an antigen and the interaction of the antigen with a specific primary antibody.

FIG. 20 is a schematic representation of iPLA₂γ translational repression constructs.

FIG. 21 is a schematic representation of the pFASTBAC vector containing gamma 23mer sequences and luciferase coding sequence.

FIG. 22 is a schematic representation of baculovirus promoter constructs containing gamma 23mer sequences and luciferase coding sequence.

FIG. 23 shows phosphorylated oligo pairs for sequences between nucleotide 364 and nucleotide 455 for use in translational repression of iPLA₂γ in the luciferase expression system.

FIG. 24 is a bar graph providing luciferase assay results showing the relative levels of expression of three different iPLA₂γ constructs in Sf9 cells.

FIG. 25 shows the N-terminal regions of mouse (SEQ ID NO: 40), rat (SEQ ID NO: 41) and human iPLA₂γ (SEQ ID NO: 42) aligned using the Internet-based MultAlign program. MultAlign does a simultaneous alignment for two or more DNA or protein sequences. The program is based on a generalization of the algorithm of M. S. Waterman, T. F. Smith and W. A. Beyer (Adv.Math. Vol. 20, pp. 367- 387 (1976)) by M.Krueger and G. Osterburg (Comp. Prog. in Biomed. Vol. 16, pp. 68- 69 (1983)).

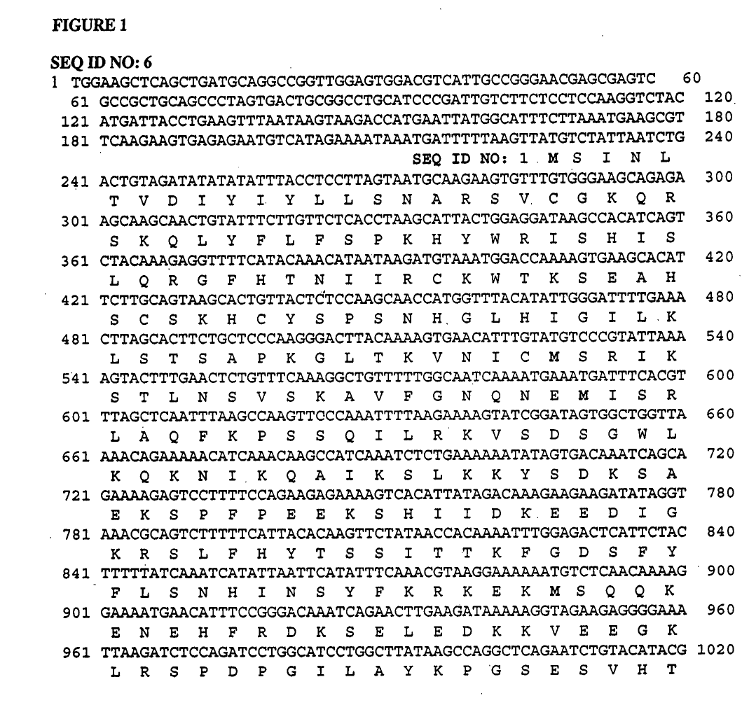

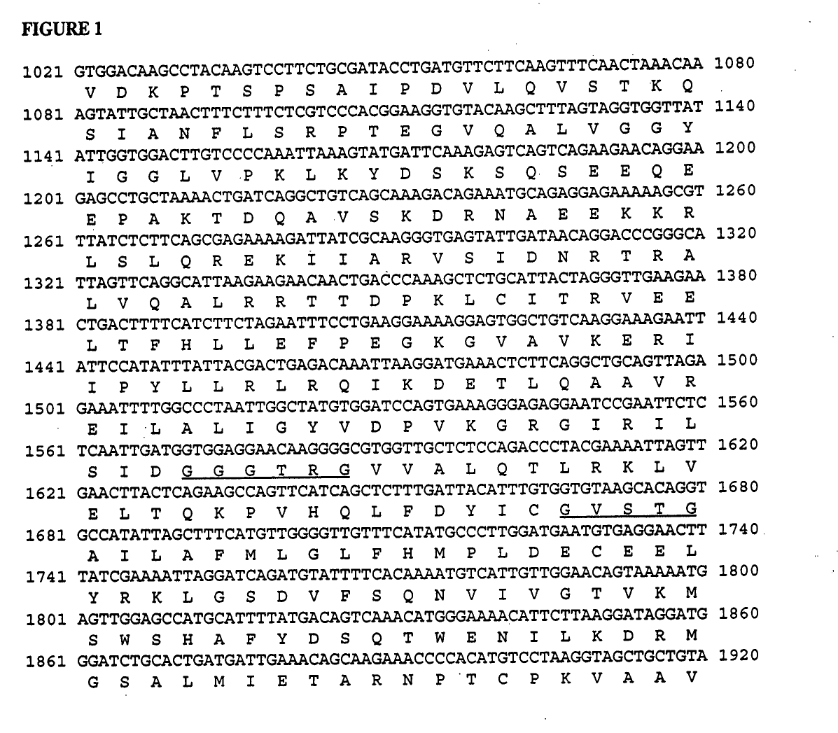

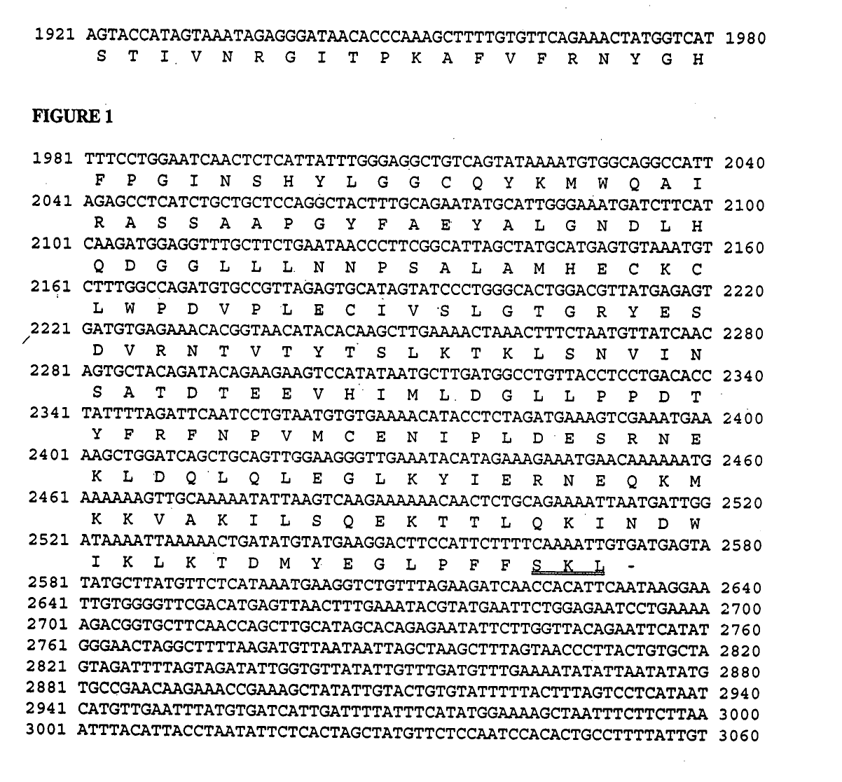

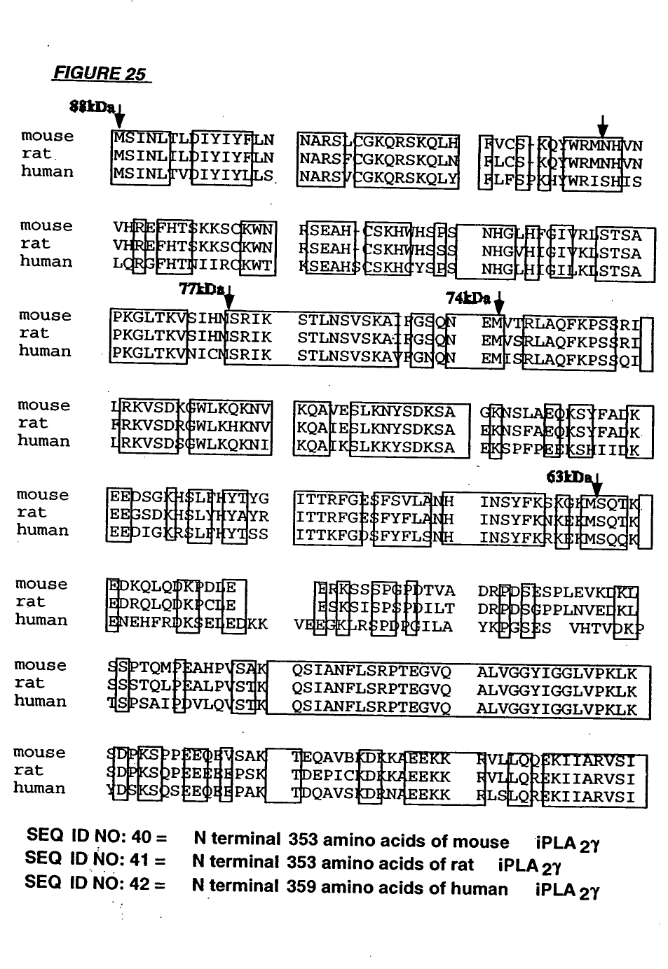

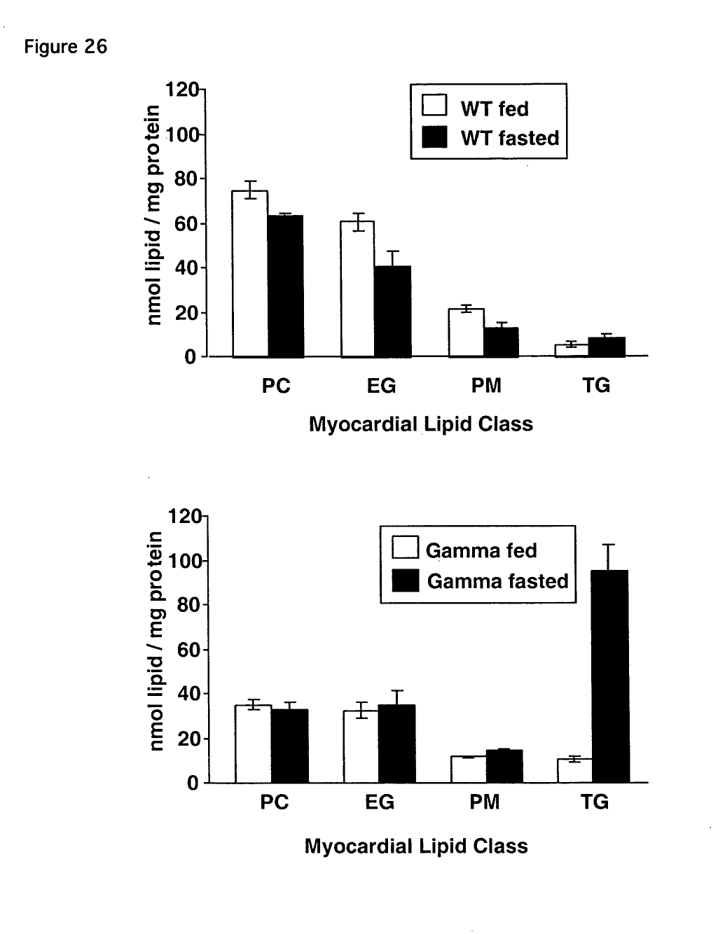

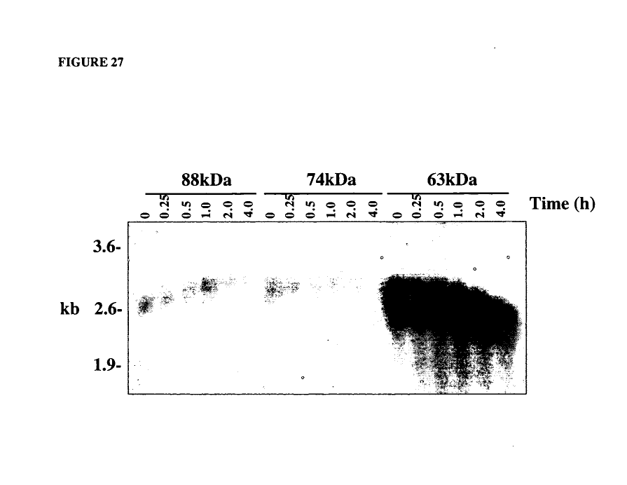

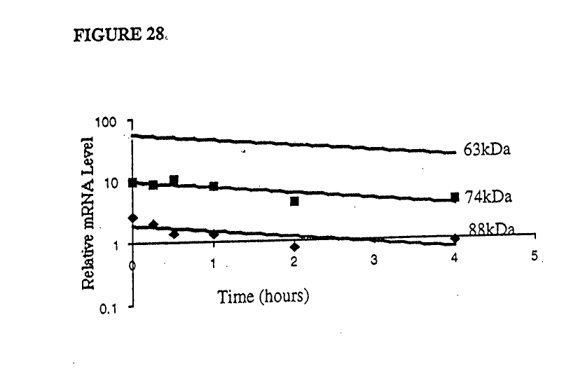

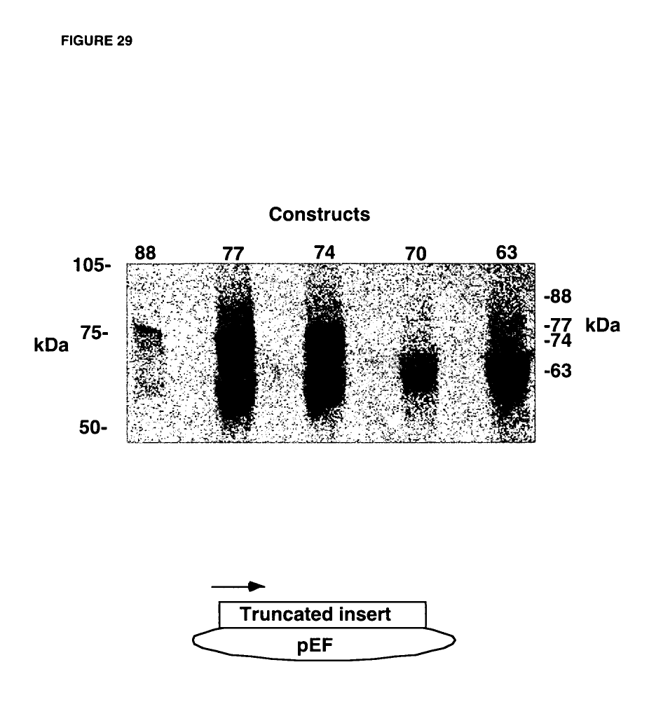

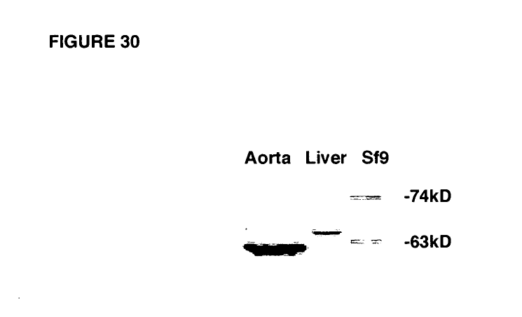

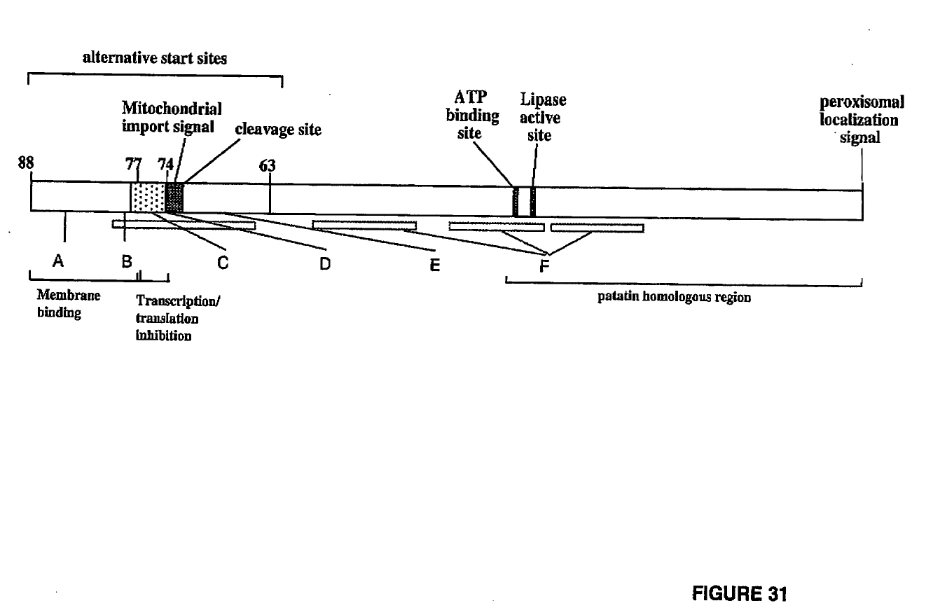

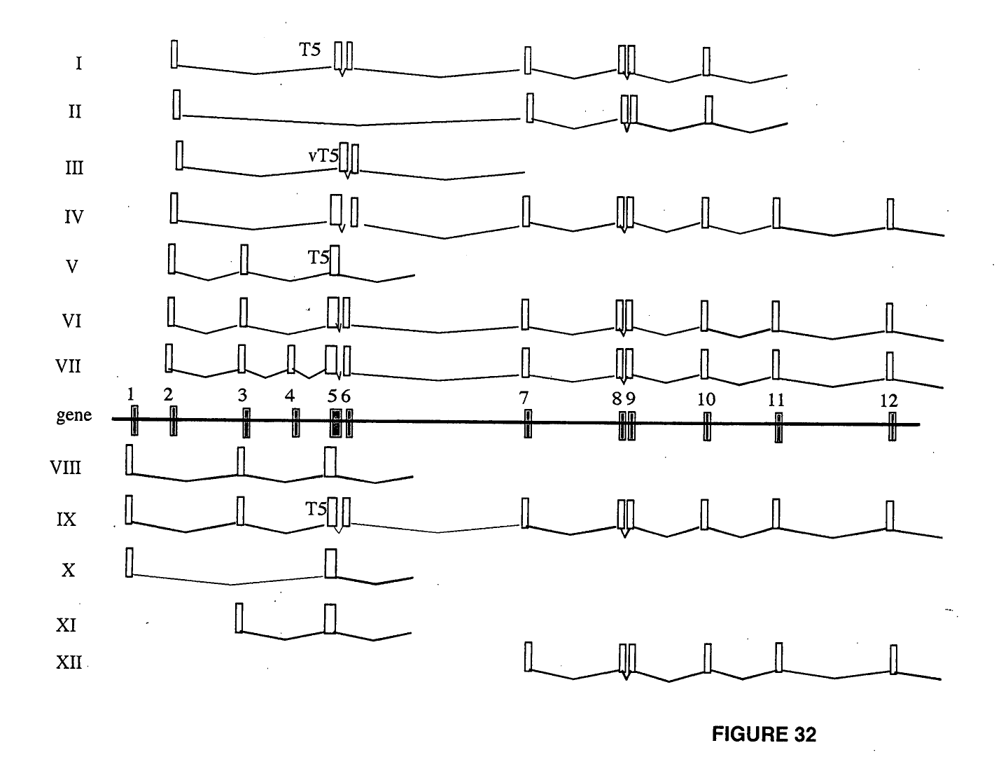

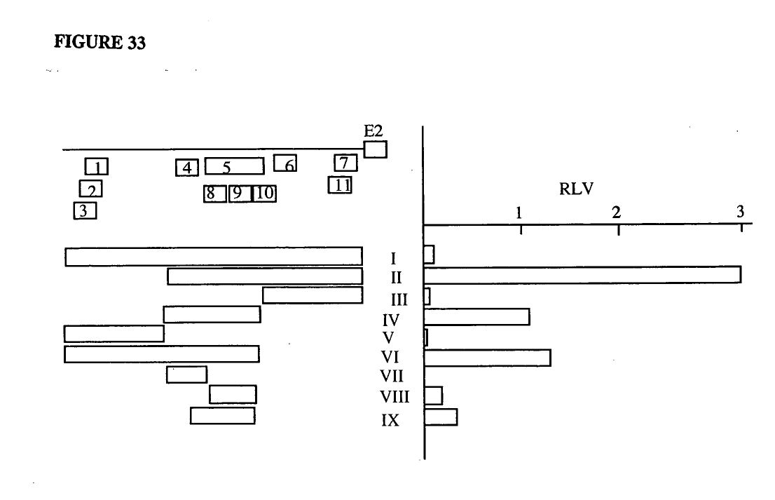

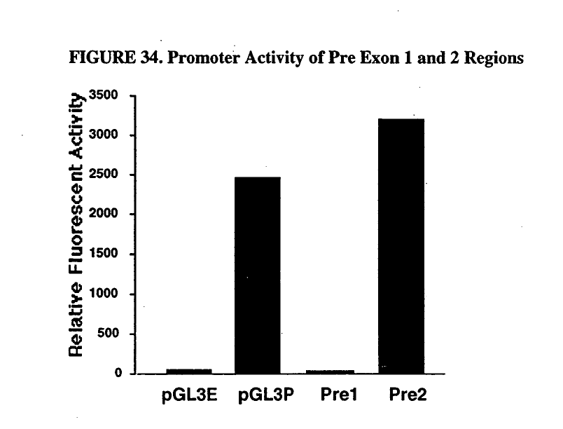

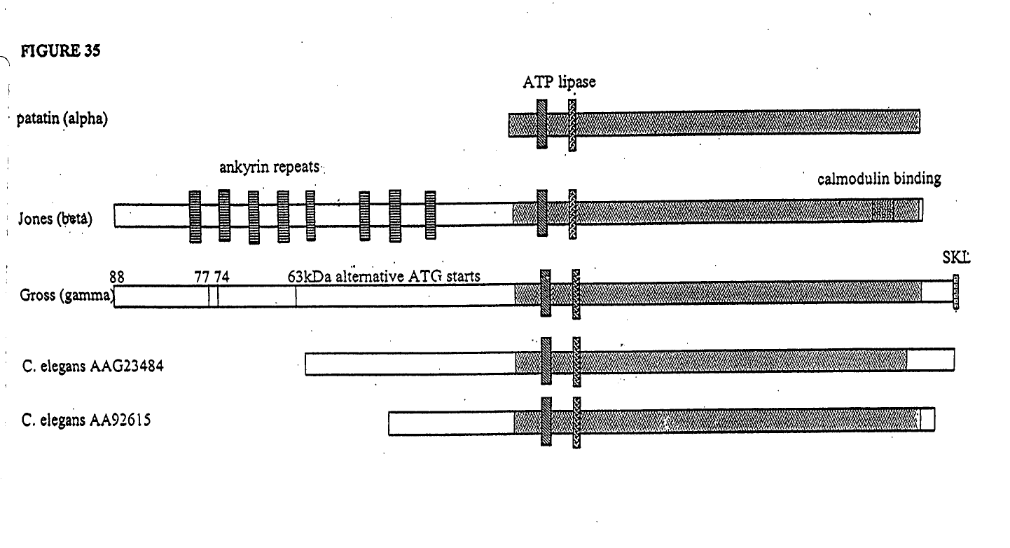

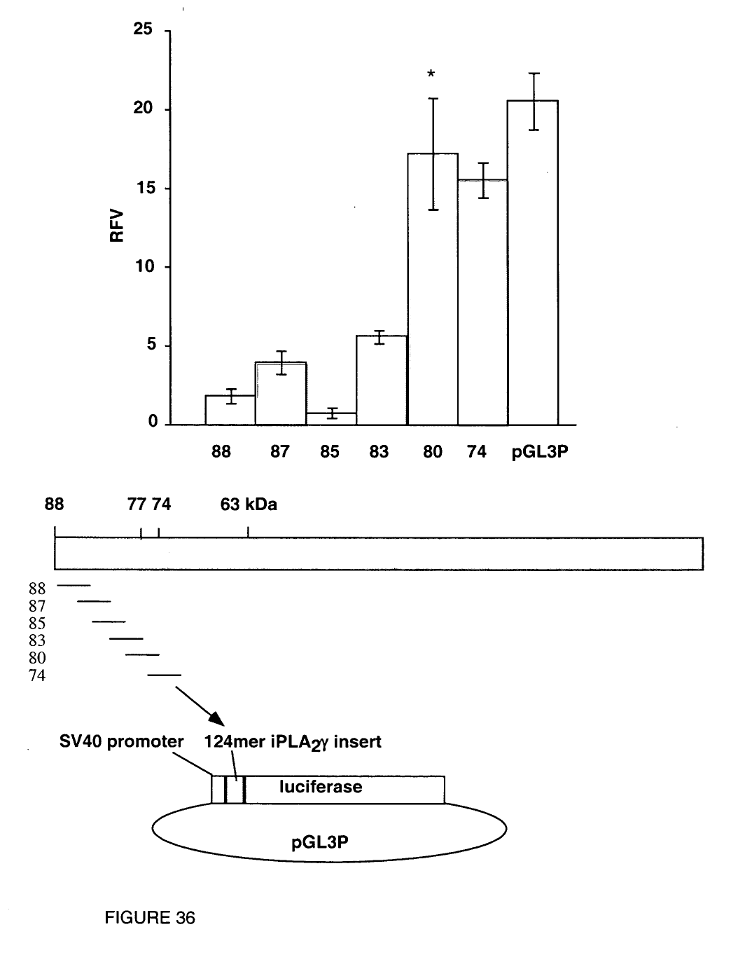

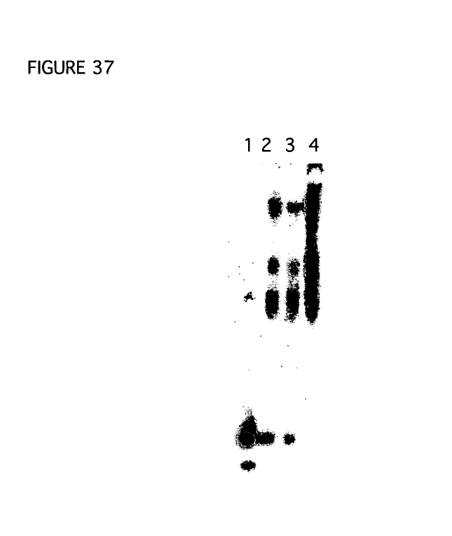

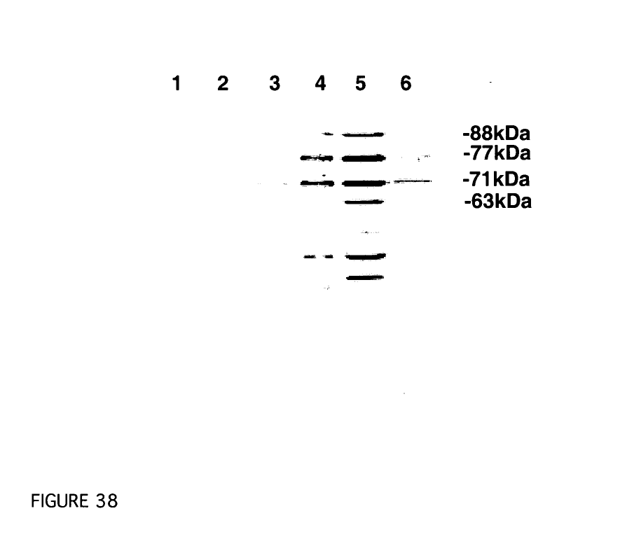

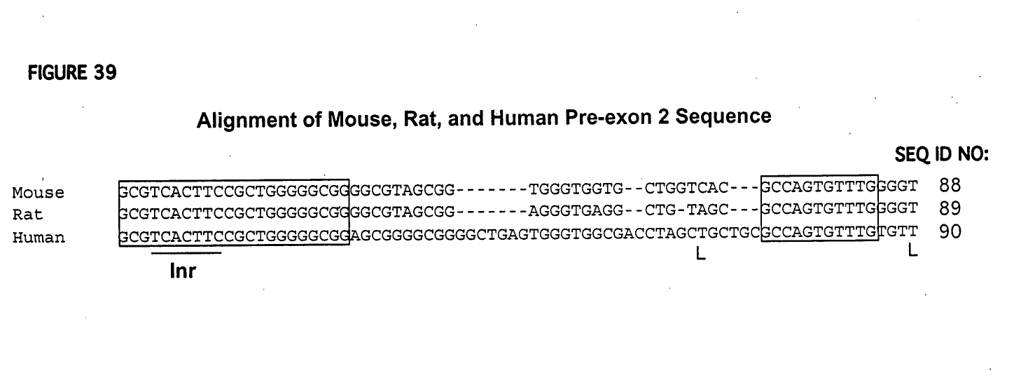

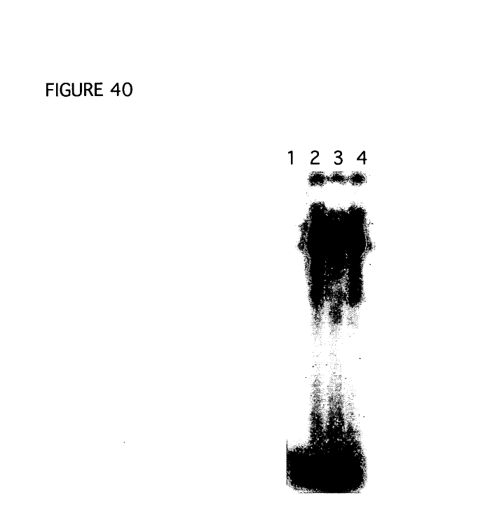

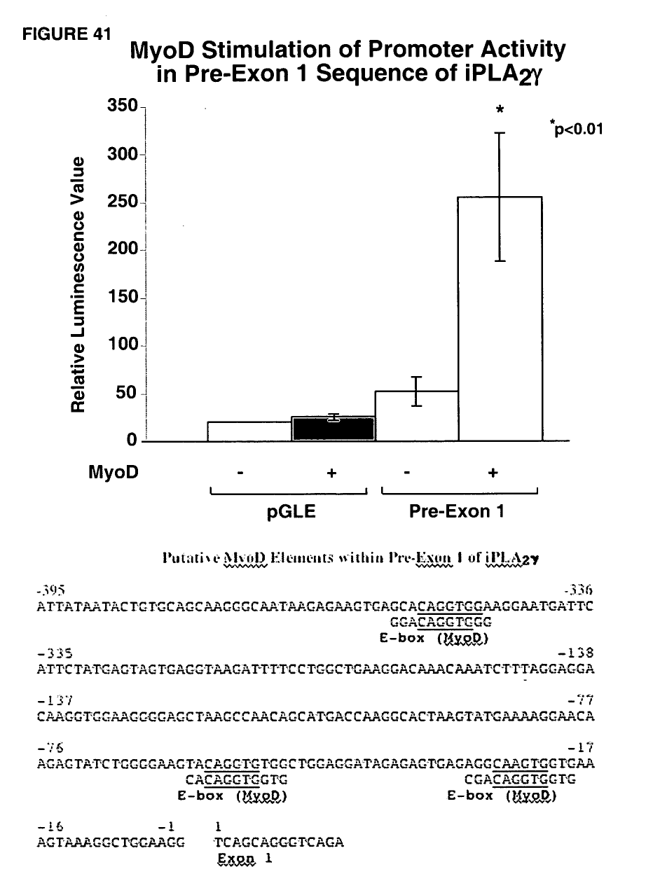

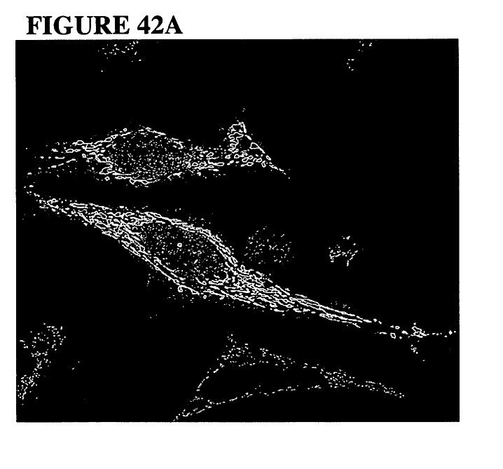

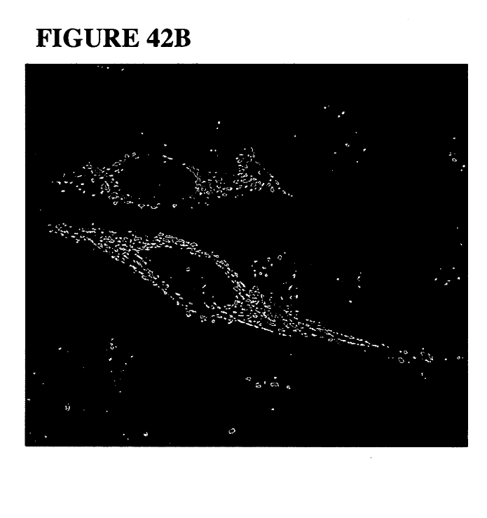

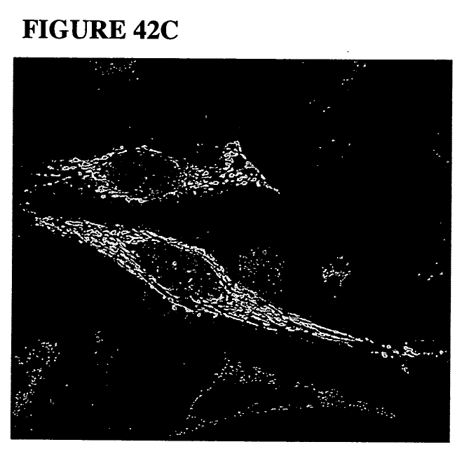

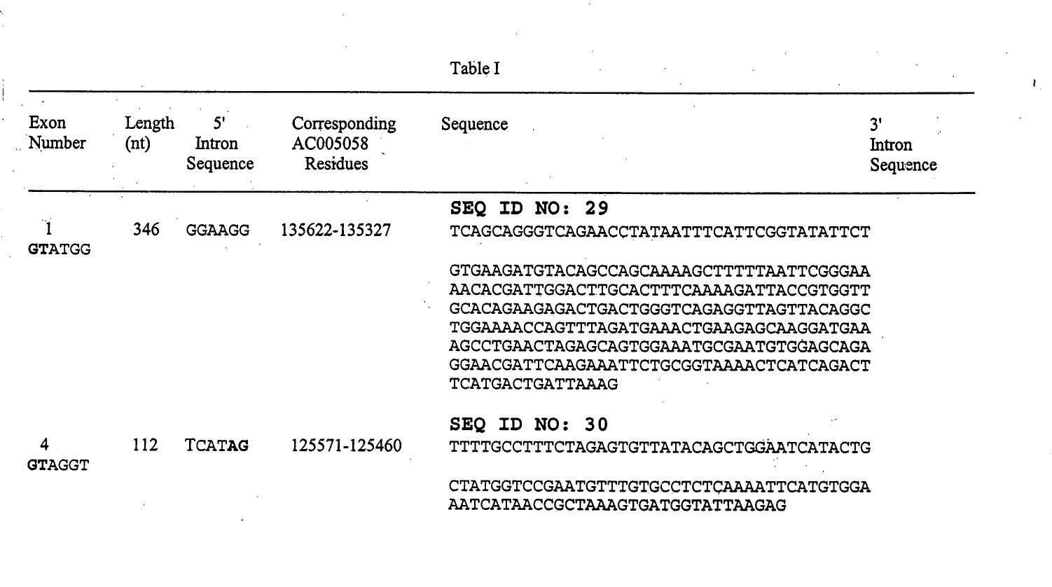

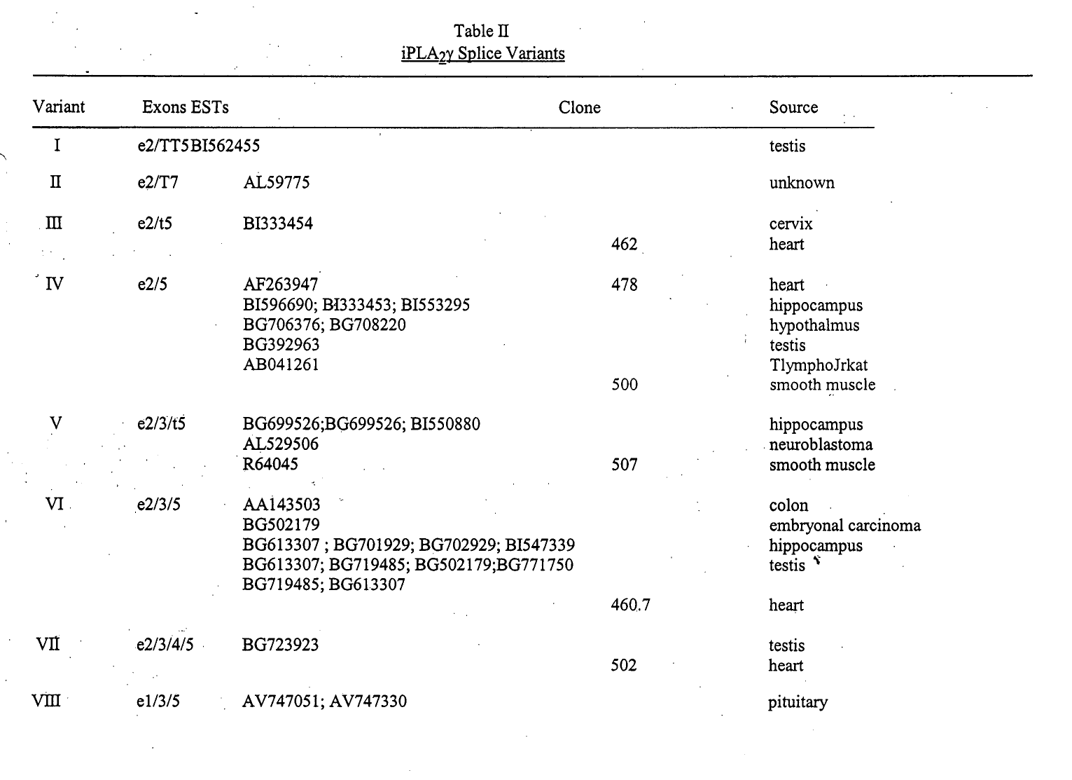

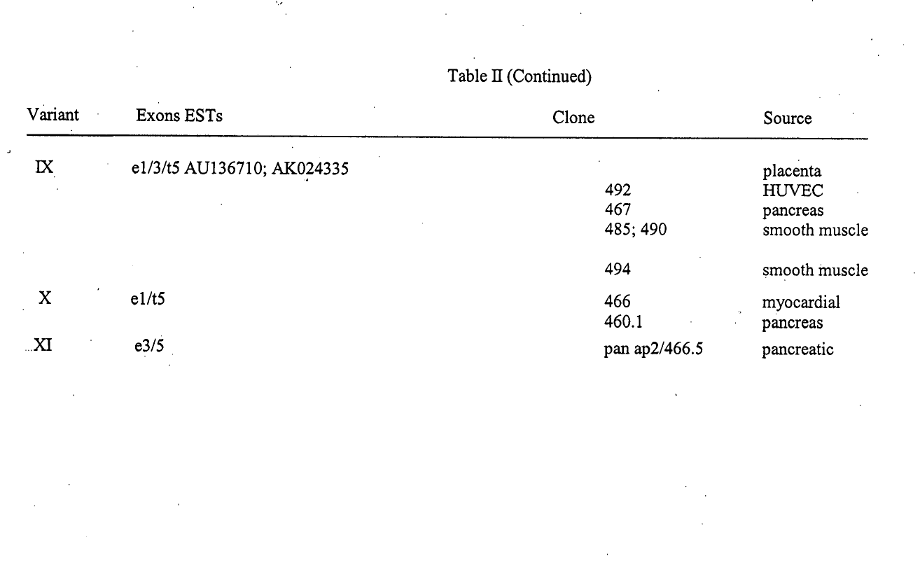

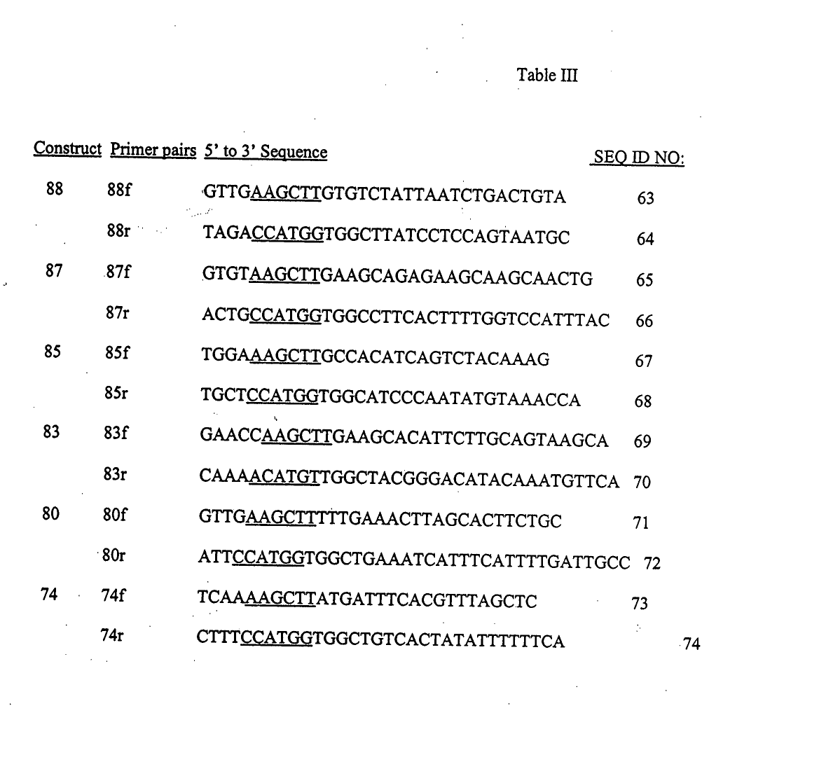

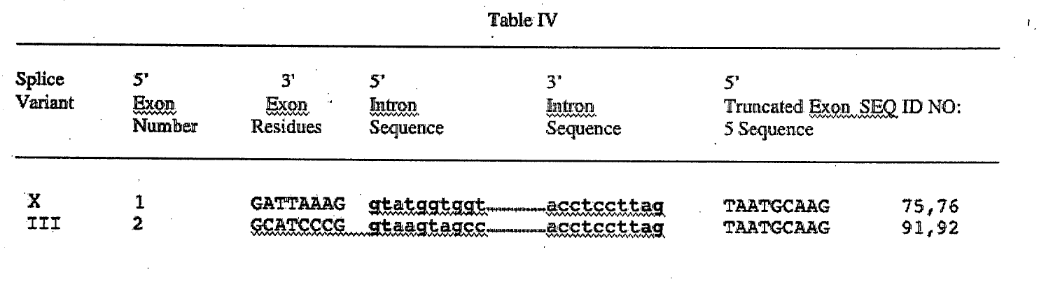

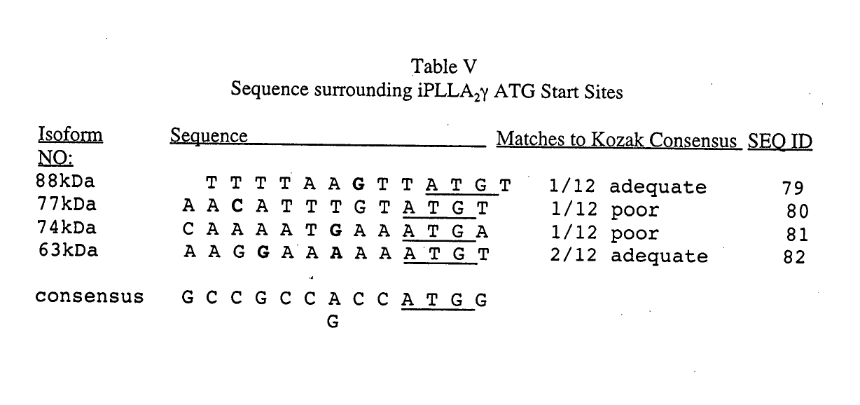

Regarding FIG. 25, arrows indicate methionine residues at which the 88 kDa, 77 kDa, 74 kDa, and 63 kDa polypeptides are encoded for the human iPLA₂γ protein. Boxes shown in FIG. 25 indicate regions of 100% conservation of amino acid sequence across all three species.

Further, regarding FIG. 25, overall, the N-terminal sequence of iPLA₂γ is highly conserved across all three species (71%). Methionine residues corresponding to the 88 kDa, 77 kDa, 74 kDa and 63 kDa polypeptides are conserved across all three species. The conservation of these methionine residues may suggest their use in the mouse and rat for downstream initiation of truncated forms of iPLA₂γ as found in the human. Interestingly, mouse and rat sequences have an additional methionine residue, not present in human iPLA₂γ, upstream from the 77 kDa start site which may potentially also be used for alternative initiation and synthesis of truncated iPLA₂γ forms.

FIG. 26 shows electrospray ionization mass spectroscopy of phospholipids in wild-type and transgenic myocardium. FIG. 26(a) shows a comparison of phosphatidylcholine, ethanolamine glycerophospholipid, plasmalogen, and triacylglyceride levels in fed (open bars) and 16 h fasted (closed bars) WT mice. P<0.01 (n=3). FIG. 26(b) shows a comparison of phosphatidylcholine, ethanolamine glycerophospholipid, plasmalogen, and triacylglyceride levels in fed (open bars) and 16 h fasted (closed bars) transgenic mice. P<0.01. In (a) and (b), other molecular species representing <2% of the total pools were also identified without demonstrable differences between control and transgenic mice.

FIG. 27 shows the result of Northern blot analysis of mRNA extracted from Sf9 cells infected for 48 h with recombinant iPLA2γ baculoviral constructs and then treated with actinomycin D for 0, 0.25, 0.5, 1.0, 2.0 and 4 hours (to resolve RNA stability). 88 kDa construct contained full length iPLA2γ coding sequence (SEQ ID NO: 6) encoding the 88 kDa protein (SEQ ID NO: 1). The 74 kDa construct contained truncated sequence (SEQ ID NO: 19) encoding the 74 kDa protein (SEQ ID NO: 18). The 63 kDa construct contained truncated sequence (SEQ ID NO: 22) encoding the 63 kDa protein (SEQ ID NO: 21).

FIG. 28 shows the observations from quantitative PCR analysis of RNA stability of truncated iPLA₂γ expression in Sf9 cells.

FIG. 29 shows expression of truncated iPLA₂γ forms in an in vitro expression system. Truncated iPLA₂γ constructs in vector pEF were expressed using a TnT® Quick Coupled Transcription Translation System and analyzed by SDS PAGE. The TnT® Quick Coupled Transcription/Translation Systems are single-tube, coupled transcription/translation reactions for eukaryotic in vitro translation. 88 kDa, 77 kDa, 74 kDa, 70 kDa, and 63 kDa truncated forms are represented as 88, 77, 74, 70, and 63. Molecular weight standards are shown on the left and the predicted corresponding sizes of each of the iPLA2γ isoforms synthesized from alternative initiator methionines are indicated on the right.

FIG. 30 shows the results of a Western blot analysis of extracts isolated from canine aorta homogenate and liver peroxisomes along with cellular extract from Sf9 cells infected with a fill-length iPLA₂γ construct. Western blotting, an extension of protein electrophoresis, is sometimes called immunoblotting and is a reliable method for checking any sample for the presence of a specific antigen. The detection is based on the molecular weight of an antigen and the interaction of the antigen with a specific primary antibody. Samples are prepared from tissues or cells that are homogenized in a buffer that protects the protein of interest from degradation. The complex protein mixture from the homogenized sample is separated by size using SDS-PAGE and then transferred to a membrane for detection with a primary antibody.

Regarding, FIG. 30, canine aorta and liver express different molecular weight forms of iPLA₂γ. Furthermore the mass of the canine aorta form is approximately 5 fold greater than the liver form. The lower band in the canine aorta lane is an artifact of the Western staining procedure since it is present even in the absence of primary (anti-iPLA₂γ) antibody. The upper band in the aorta lane is approximately 60 kD while the liver form is approximately 66 kD. While the expressed forms do not correspond in mass to the expressed forms from the baculoviral expression system, the significance of these results is three fold: 1) These results represent the first reported detection of endogenously expressed iPLA₂; 2) These results demonstrate that iPLA₂γ is differentially processed to different molecular masses in different tissues; and 3) These results demonstrate that in the endogenous state, the iPLA₂γ isoforms are expressed to different degrees in different tissues. The significance of the latter two findings is that these results show, that iPLA₂γ is a molecule which undergoes regulated expression (resulting in truncated forms and differential expression). Several mechanisms are disclosed herein by which this regulation can occur (alternative splicing, alternative promoter usage, and use of alternative initiator methionines). Regulation by these means has been demonstrated in artificial expression systems (baculoviral expression or transfection of cells with truncated constructs). These results clearly show that a similar regulation also occurs endogenously. Undoubtedly, additional means of regulation are also used endogenously, e.g. phosphorylation, myristylation, or proteolysis.

FIG. 31 shows a diagrammatic representation of the functional domains of iPLA₂γ. The dark rectangle represents the full-length iPLA₂γ protein (SEQ ID NO: 1). The locations of alternative initiator methionine residues within the region “alternative start sites” and encoding 88 kDa, 77 kDa, 74 kDa, and 63 kDa polypeptides are indicated as 88, 77, 74, and 63 respectively. The N-terminus of the protein is also identified as the membrane binding region due to the hydrophobicity of this region of the polypeptide. Within the “alternative start site” region is the putative “inhibitory region” C-terminal of the 74 kDa start site. The ATP binding motif, lipase consensus site, and peroxisomal localization signal site within the “patatin homologous region” are also identified. Amino acid number is indicated by the scale at the top. Sequences are shown in SEQ ID NOS 95-103, left to right, respectively, starting from the top.

A predicted mitochondrial import signal region is shaded and the putative cleavage site (LRK/VS) (SEQ ID NO: 95) is indicated. This import signal and cleavage site were predicted by the internet program Mitoprot (http://www.mips.biochem.mpg.de/cgi-bin/proj/medgen/mitofilter). In a preliminary study we have shown that in vitro synthesized product corresponding to the 74 kDa protein is cleaved to a smaller protein (approximately 72 kDa) in a mitochondrial uptake assay. These results lend support to our prediction that the 74 kDa iPLA₂γ has a mitochondrial import signal which is cleaved during import into the mitochondria. Based on this information, iPLA₂γ, appears to be a protein which has both a peroxisomal as well as a mitochondrial localization. The regulation of the intracellular localization of iPLA₂γ has important implications in regard to its potential role in modulation of cellular functions. For example, a peroxisomal localization may imply utilization of lipids for heat generation in uncoupling ad free radical generation while a mitochondrial localization may suggest that lipids released through iPLA₂γ action are utilized for cellular energy metabolism and function in the cell or coupling of mitochondrial oxidation and thermogenesis by uncoupling in the mitochondrial membrane. It should also be specifically pointed out that the actions of iPLA₂γ on membrane lipids likely affects the function of membrane associated proteins such as ion channels which can dramatically affect cellular function. Other computer predicted sites of interest are also indicated including a zinc finger, Kringle site, SAP putative DNA binding motif, membrane retention signal, amidation site, and putative M-protein repeats. These significance of these predicted domains remains to be elucidated by further studies including expression of site directed mutated forms of iPLA₂γ.

FIG. 32 shows a total of twelve potential splice variants of iPLA₂γ. A representation of the gene sequence is shown in the middle of the diagram with exons 1-13 (indicated as boxes) numbered in the diagram. Splice variants 1-XII are represented by the exons they contain (boxes) connected by lines which represent the splicing out of intron sequences. For example, splice variant I contains exon 2, a truncated exon 5 (T5), and exons 6, 7, 8, 9, and 10. A line extends beyond exon 10 to indicate the presence or absence of exons 11-13 are unknown at the present. Incomplete sequence is available for variants I, II, III, V, VIII, X, and XI, In contrast, the full sequence of splice variant IV is known and it contains exons 2,5,6,7,8,9,10,11,12, and 13.

These results suggest that two basic types of splice variants of iPLA₂γ exist, those initiating from exon 1 and those initiating from exon 2. Thus far, no splice variants have been identified containing both exons 1 and 2 either through analysis of sequences within GenBank® or by RTPCR analysis performed in our laboratory. These results suggest that iPLA₂γ utilizes two different promoters upstream of exons 1 and 2 respectively. That many splice forms of iPLA_{2γ} have been identified supports our belief that endogenous synthesis of iPLA₂γ is a highly regulated process.

FIG. 33 shows the analysis of promoter activity of the iPLA₂γ nucleotide sequence upstream from exon 2.

FIG. 34 shows promoter activity of the pre exon 1 and 2 regions. Sequence corresponding to approximately 600 nt upstream of exons 1 and 2 were PCR amplified from the genomic BAC clone AC005058 and cloned upstream of the promoterless vector, pGL3E (Promega Corporation, 2800 Woods Hollow Road, Madison, Wis. 53711) to drive expression of the luciferase gene when transfected into CV1 cells. Pre exon 2 sequence had high promoter activity compared to the pGL3P control vector containing SV40 promoter. In contrast, the pre exon 1 sequence had negligible promoter activity as did the pGL3E promoterless negative control vector. These results suggest that pre exon 2 sequence is used endogenously for expression of iPLA₂γ initiating from exon 2. The presence of sequences initiating from exon 1 suggests that sequences upstream from exon 1 are an alternative promoter region for iPLA₂γ expression. These results provide further evidence that the iPLA₂γ expression is regulated by alternative promoter usage. It is easy to speculate that pre exon 1 promoter sequence may be required under specific cell conditions such as differentiation or under conditions of stress. The lack of demonstrable promoter activity from pre exon 1 sequence may suggest that additional specific transcription factors may be required to initiate transcription in the CV1 system or may indicate that additional upstream sequences are required for promoter activity.

FIG. 35 shows the homologies between the three members of the patatin family of proteins: potato patatin (alpha), the iPLA₂β protein described in U.S. Pat. No. 5,976,854 which issued to Simon Jones, et al. on Nov. 2, 1999 (beta), and the iPLA2γ protein described and claimed in this application (gamma). Also shown are representations of two C. elegans protein sequences reported in GenBank® which are iPLA_{2γ} homologs. All the proteins represented contain a common region of homology to patatin (stippled box). Within this region are ATP and lipase consensus sites as indicated by the shaded boxes. That these proteins share a patatin domain has important implications with regard to their evolution, strongly implying that all were evolved from a common ancestor related to potato patatin and eventually diverged into three separate classes of iPLA₂ molecule, alpha, beta, and gamma.

In addition to the patatin domain, iPLA2β (beta) has eight ankyrin repeats and a calmodulin binding domain. iPLA₂γ has 4 alternative ATG start sites at which proteins of 88, 77, 74, and 63 kDa can be synthesized. iPLA₂γ also has a C-terminal SKL peroxisomal localization sequence. To further distinguish iPLA₂β from iPLA₂γ, it should further be pointed out that: 1) They do not share any significant homology in their N-terminal halves; 2) They are encoded by separate genes on different chromosomes (iPLA₂γ is on 7 while iPLA₂β is on 22); 3) Their respective genes contain different numbers of exons (13 for iPLA₂γ and 16 for iPLA₂β); 4) That, unlike iPLA₂β, iPLA₂γ has two alternative promoters; and 5) iPLA₂β and iPLA₂γ have different intracellular localizations (membrane associated and peroxisomal for iPLA₂γ vs. mitochondrial and primarily cytosolic for iPLA₂β). Like iPLA₂γ, the C. elegans proteins contain the patatin region of homology with ATP and lipase consensus sites and they do not contain ankyrin repeats. Blast analysis indicates that, in terms of amino acid homology, these C. elegans sequences are most like iPLA₂γ and thus are likely to represent the C. elegans homolog of human iPLA_{2γ}and belong to the class of molecules designated ‘gamma’ or iPLA₂γ.

The identification of any new or otherwise unknown sequence from sequencing databases as being an iPLA₂γ is made by performing NCBI Blast analysis (http://www.ncbi.nlm.nih.gov/BLAST/) of the sequence in question. Identity or a high degree of alignment with our reported GenBank® sequence (accession number AF263613 or with SEQ ID NOS 3, 4, 5, 6, 13, 16, 19, 22) will characterize the sequence as iPLA₂γ or a homolog thereof. FIG. 25, for example shows the homology between mouse, rat, and human iPLA₂γ. These sequences are all found in the GenBankg database. Boxed areas show amino acids which are identical between the three species.

FIG. 25 reveals the high degree of N-terminal similarity of iPLA_{2γ} from these three species. Within the first 210 amino acids, 145 amino acids are precisely conserved (69%). Comparing the full-length mouse iPLA₂γ sequence (GenBank® accession number NP_{—}080440), there is 82% identity of amino acid sequences when compared with the human sequence (SEQ ID NO: 1). If conservation of amino acid type is included, the homology rises to 90%. The differences in the sequences represent species differences. Nonetheless, all three sequences are clearly iPLA₂γ and likely have the same or very similar functional attributes. In contrast, a similar comparison reveals only 38% homology of mouse NP_{—}080440 to human iPLA₂β.

Other species specific homologs of iPLA₂γ exist with lower homology. The C. elegans polypeptide, GenBank® accession number NP_{—}500969 shares 57% homology with human iPLA₂γ (SEQ ID NO: 1) while C. elegans polypeptide, GenBank® accession number AAF39894, shares 38% homology. And each is more homologous with iPLA₂γ than iPLA₂β sequence. Furthermore, NP_{—}500969 contains sequence which is an identical match at the human iPLA2γ lipase ICGVSTG (SEQ ID NO: 52) and ATP binding LSIDGGGTRG (SEQ ID NO: 53) domains (where underlined amino acids are also identically conserved in other polypeptides containing these domains). And so, in all likelihood, sequence NP_{—}500969 represents the C. elegans homolog of human iPLA₂γ. Phylogenetic analysis suggests that a common ancestor gave rise to three main branches, the patatin, iPLA₂β and iPLA₂γ homologs. The iPLA₂γ branch includes the highly homologous human, mouse, and rat sequences as well as less homologous sequences from C. elegans (AAG23484, CAA92615, T33857, AAK21368, and AAF39894) and Plasmodium falciparum (AAC71877). As of yet, only the human sequence has been expressed and has calcium independent phospholipase A₂ activity. However, each of these, with the exception of AAC71877 and AAF39894 contains closely linked regions with perfect matches to the lipase (GxSxG) and ATP binding (GxGxxG) consensus sequences suggesting that these sequences represent authentic iPLA₂γ in other species.

FIG. 36 illustrates the identification of a transcriptional regulatory domain within the 5′ coding region of iPLA₂γ using a luciferase reporter assay system.

FIG. 37 illustrates gel mobility shift analysis (gel shift) of the iPLA_{2γ} transcriptional regulatory domain within the 5′ coding region of iPLA₂γ for identification of a nuclear binding domain utilizing oligonucleotide CdxA (SEQ ID NO 59).

FIG. 38 illustrates Immunoblot analysis of iPLA₂γ in subcellular fractionations of rat heart showing differential expression of iPLA₂γ isoforms in different subcellular fractions.

FIG. 39 illustrates alignment of mouse (SEQ ID NO: 88), rat (SEQ ID NO: 89), and human (SEQ ID NO:90) pre-exon 2 sequence identifying an initiator (Inr) site as well as a putative core promoter element.

FIG. 40 illustrates competitive gel retardation analysis of iPLA₂γ regions identifying a transcription factor binding site Inr defined by SEQ ID NO: 62.

FIG. 41 illustrates MyoD stimulation of promoter activity in pre-exon 1 sequence of iPLA₂γ by measuring luciferase expression following transfection of CV1 cells with promoterless vector (pGLE) or with a construct in which pre exon 1 sequence is inserted upstream of the luciferase gene in pGLE (pre-exon 1 with (+) or without (−) cotransfection with myoD vector. The full length DNA sequence is shown in SEQ ID NO: 84, while the E-box (MyoD) oligonucleotides are shown in SEQ ID NOS 85-87, respectively, in order of appearance.

FIG. 42-A,B,C illustrates import of GFP into mitochondria mediated by an N-terminal iPLA₂γ mitochondrial import signal in HeLa cells transfected singly or cotransfected with 74 GFP and DSRed2-Mito constructs and visualized by confocal microscopy.

We demonstrate herein: 1) the presence of nuclear protein constituents which bind to the repressor region of the 5′ region of iPLA₂γ by gel shift analysis; 2) tissue specific translational regulation of iPLA₂γ; 3) the presence of a mitochondrial localization signal in the N-terminus of the major heart iPLA₂γ isoforms. The presence of dual localization signals in discrete iPLA₂γ isoforms regulated by tissue-specific genetic programs identifies a heretofore unanticipated complex interplay between cardiac mitochondria and peroxisomes for the uptake and utilization of the nascent iPLA₂γ protein. Since the rate determining step in mitochondrial uncoupling protein (UCP) function is the interaction of mitochondrial non-esterified fatty acids with UCP, the results identify a diverse repertoire of iPLA₂γ proteins which likely serve as important metabolic determinants integrating mitochondrial respiration and thermogenesis.

In summary, human iPLA2γ is synthesized from a single gene made up of 13 exons on chromosome 7q31. Naturally occurring polymorphisms are likely to result in a small percentage of nucleotide or amino acid substitutions (probably less than 1%). On the other hand, alternative splicing of the gene results in at least 10 different splice variants. Since the splicing occurs at the 5′ end of the gene and there is no evidence that splicing involves the lipase active site, each of the splice variants potentially has phospholipase A2 activity. Alternative splicing is a well recognized mechanism of modulation of protein function (Leff, et al. Annual Rev Biochem 55: 1091-1117, 1986) and can affect many cellular processes such as signal transduction, transcriptional regulation, cellular transformation, and subcellular localization. A number of genetic defects have been associated with incorrect splicing. Alternative splicing of iPLA₂γ may thus be a functional mechanism for regulating iPLA₂γ activity in cells in terms of both the amount of activity as well as the specific intracellular sites at which the activity occurs. Further modulation occurs in the form of truncation by use of downstream alternative ATG start sites resulting in 77 kDa, 74 kDa, and 63 kDa polypeptides. This truncation is also likely to have important functional consequences. Indeed, we have shown that transgenic mice generated using a construct containing SEQ ID NO: 6 which encodes the 88 kDa protein (SEQ ID NO: 1) express 74 kDa and 63 Da proteins (SEQ ID NOS 18 and 21). Additionally, we showed that canine iPLA2γ (which cross reacts with antibodies to human iPLA₂γ) is endogenously expressed as two different isoforms. Homologs of iPLA₂γ have been identified in other species besides canine including rat, mouse which share as much as 90% homology with human iPLA₂γ, and in C. elegans which share approximately 38-57% homology with human iPLA_{2γ}.

Three additional exons in the iPLA₂γ gene have been identified. For identification of these exon sequences, we used the same approach employed in our publication (Mancuso et al. J. Biol. Chem. 275: 9937-9945, 2000), alignment of raw genomic and coding (EST) sequences in GenBank® as well as comparison of these sequences with sequences cloned in our laboratory from 5′ RACE of human cDNA libraries and identification of 5′ and 3′ splice signals.

These new exon sequences are shown in Table 1. It should be appreciated that these sequences were present in GenBank® as “raw” sequence within a much larger submitted block of sequence. Within this sequence, they were not identified as being iPLA₂γ sequence nor were they identified as exons or introns nor were the 5′ and 3′ boundaries noted, nor were these sequences identified as having any potential functional or otherwise significance other than being human genomic or coding sequence. We now identify and characterize these sequences as being exons within the iPLA_{2γ} gene and present detailed information in Table 1 including exon number, length of each exon, the 5′ intronic sequence including the ‘ag’ splice signal for exon 4, nucleotide sequences of exon 1 and 4 (SEQ ID NOS: 29 and 30, respectively), and 3′ intron sequence with the ‘gt’ splice signal bolded.

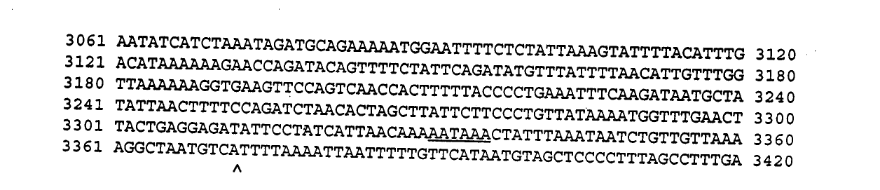

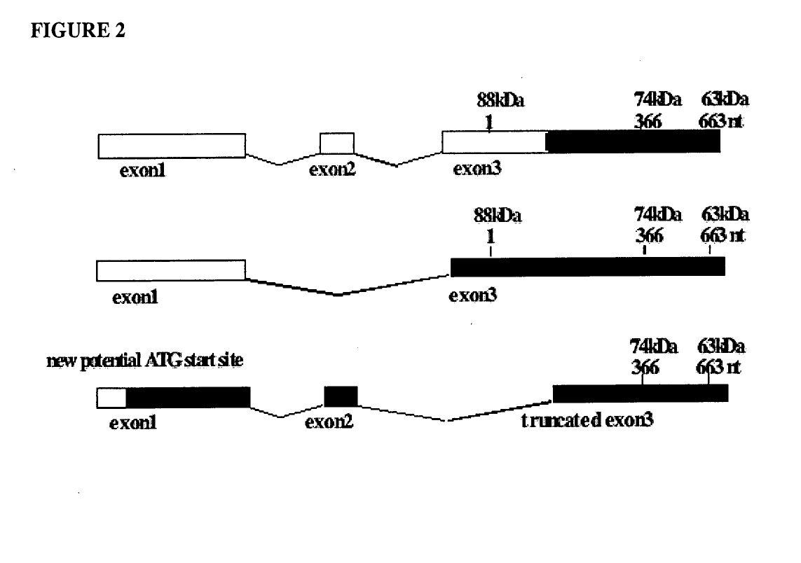

Table II shows that, in addition to the three splice variants described in FIG. 2, we have identified an additional eight (8) iPLA2γ splice variants (for a total of eleven (11) splice variants) through examination of the GenBank® databases as well as by cloning and sequencing of RTPCR in our laboratory.

Table III illustrates PCR Primers (SEQ ID NO: 63-74) for analysis of iPLA₂γ regulatory domain.

Table IV illustrates splice junctions of iPLA₂γ splice variants III (SEQ ID NOS: 91-92) and X (SEQ ID NOS: 75-76).

Table V illustrates sequence surrounding iPLA₂γ AUG start sites (SEQ ID NO: 79-82) in comparison with Kozak consensus sequence (SEQ ID NO: 83).

### DETAILED DESCRIPTION OF THE INVENTION

The present invention is based upon our identification, isolation and sequencing (characterization) of the novel calcium-independent phospholipase A₂, iPLA_{2γ}. The new iPLA₂γ is both membrane-associated and highly sensitive to inhibition by (E)-6-(bromomethylene)-3-(1-napthalenyl)-2H-tetrahydropyran-2-one (BEL), which distinguishes our new iPLA₂γ from previously reported membrane-associated phospholipases A₂. Prior to this invention, iPLA_{2γ} was unknown and had not been distinguished as a biologically active substance discrete from known calcium-independent phospholipases A₂, nor had it been isolated in pure form and its putative sequences determined.

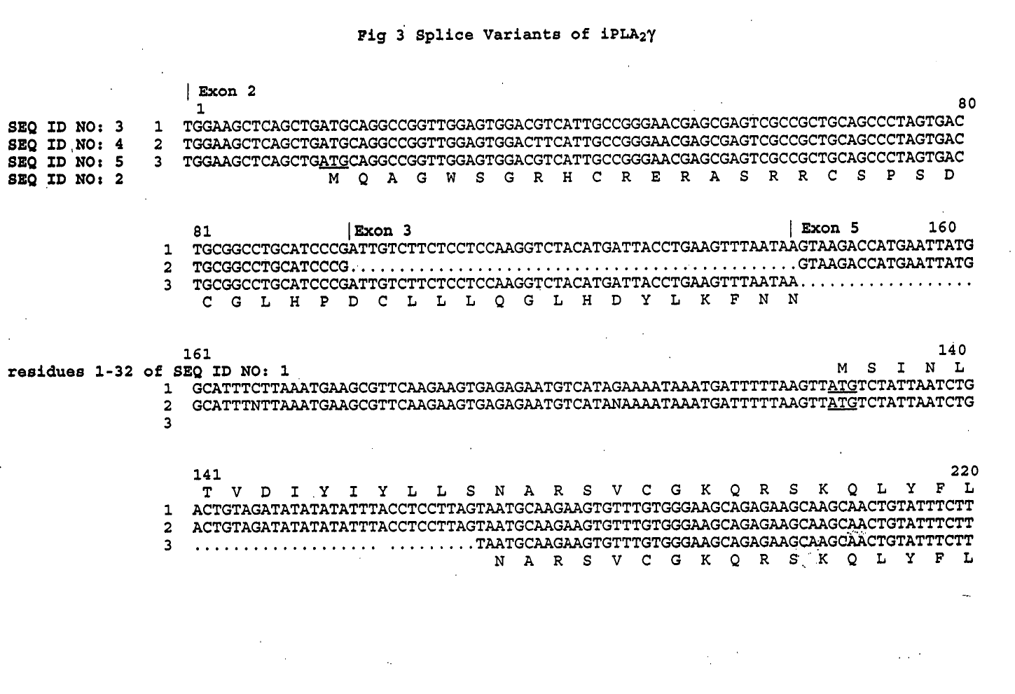

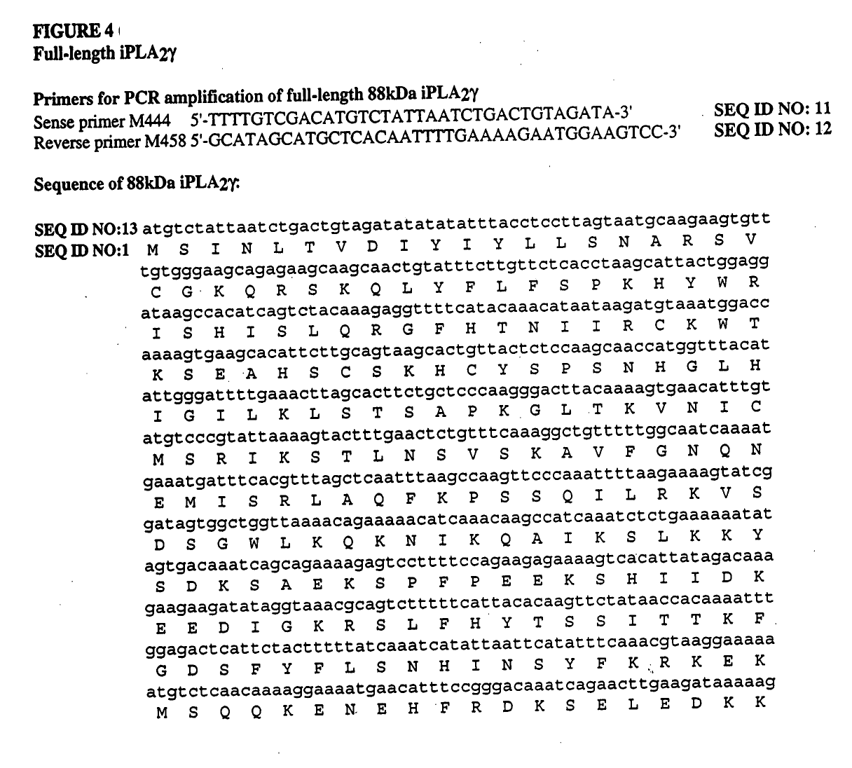

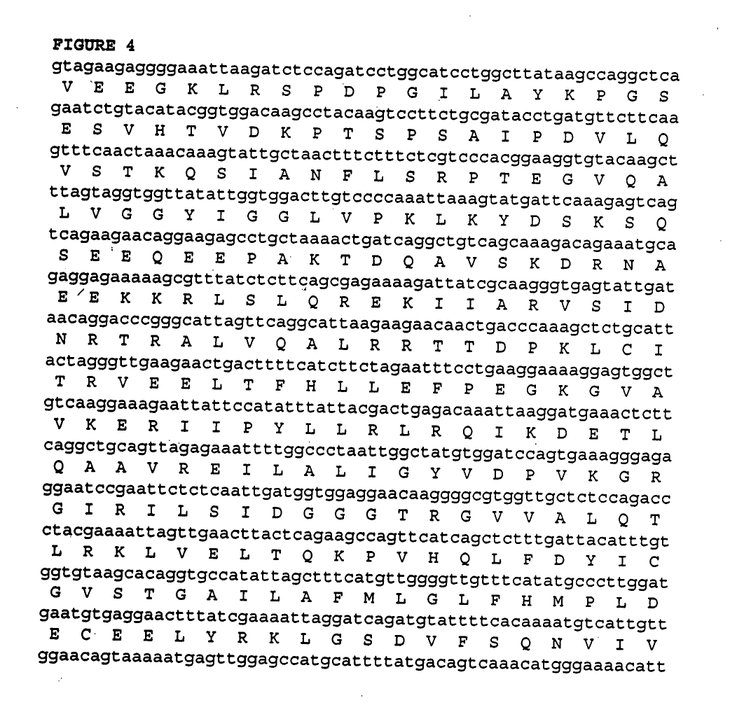

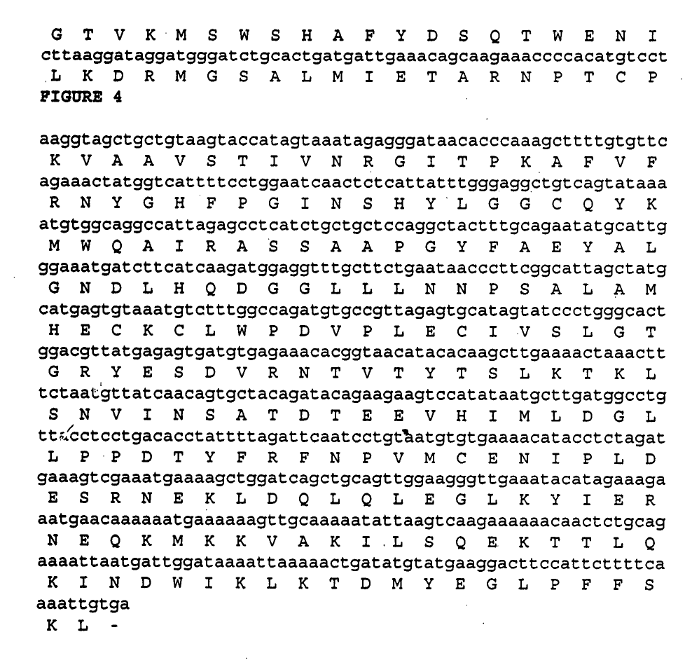

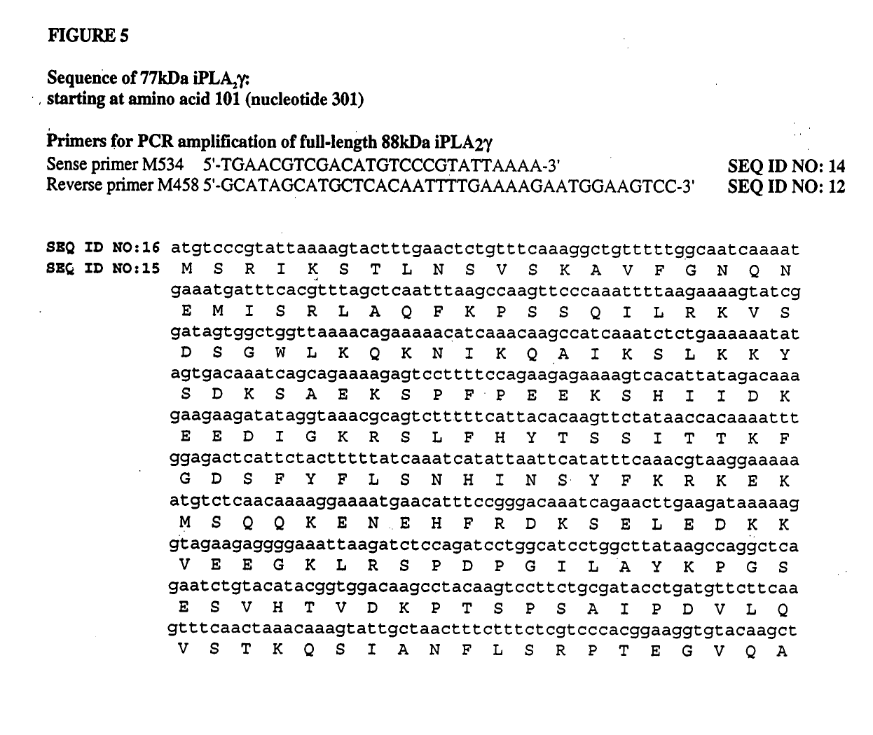

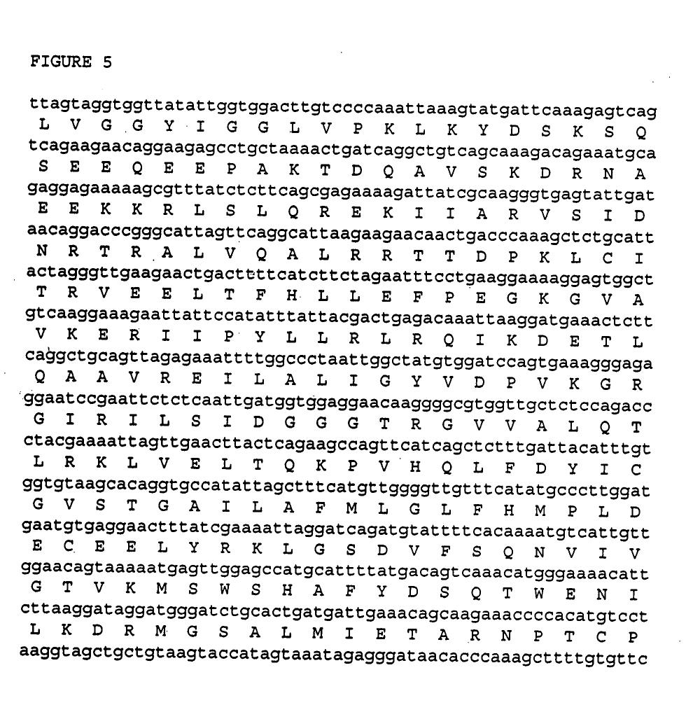

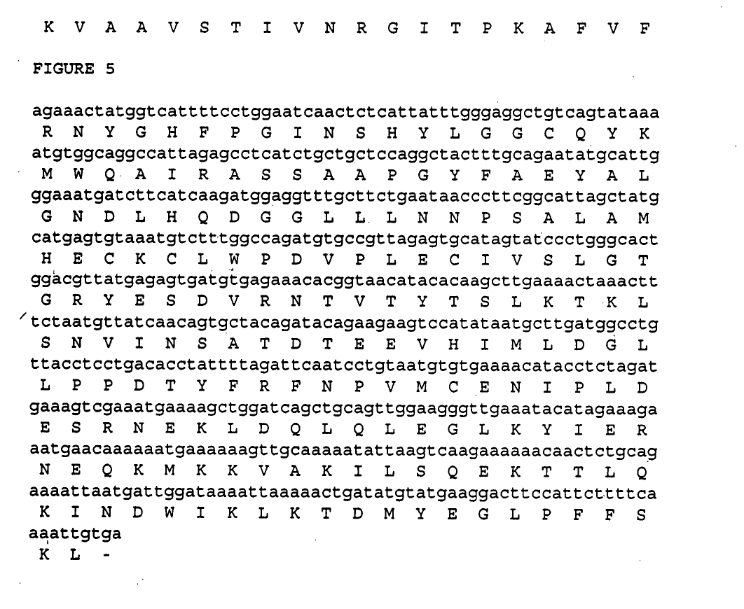

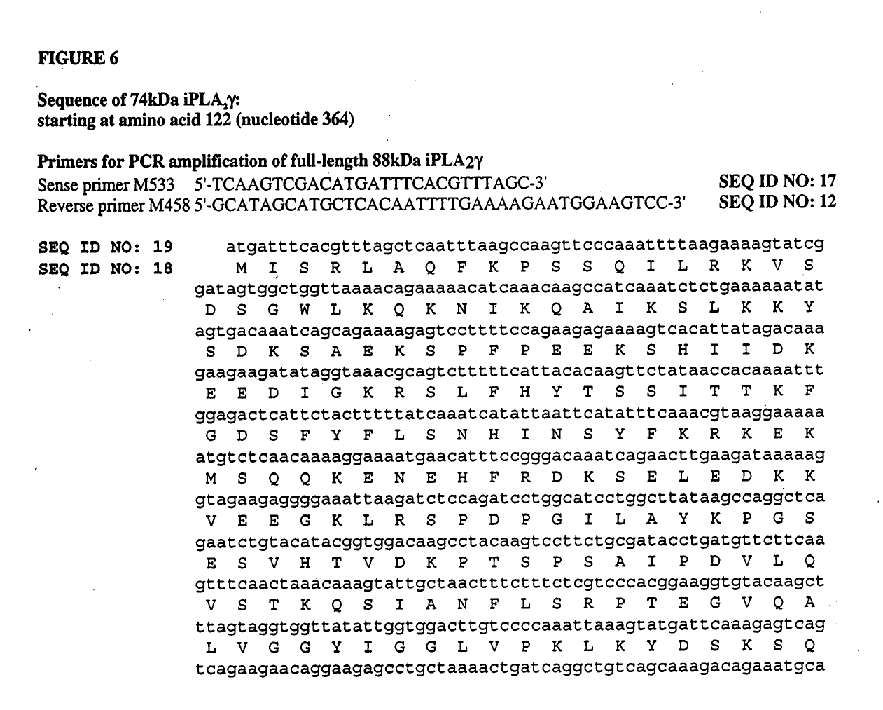

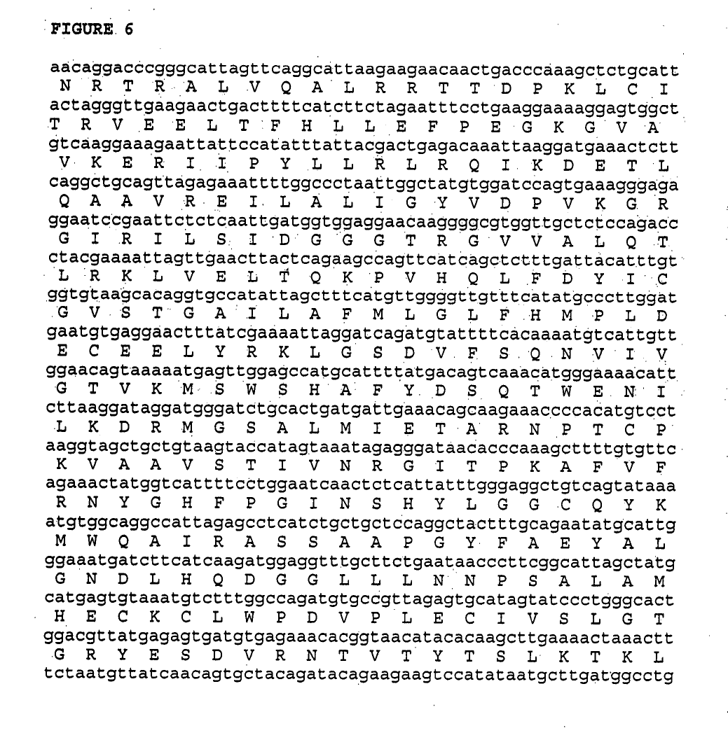

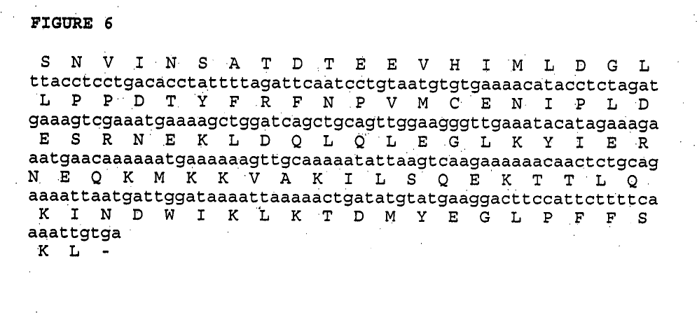

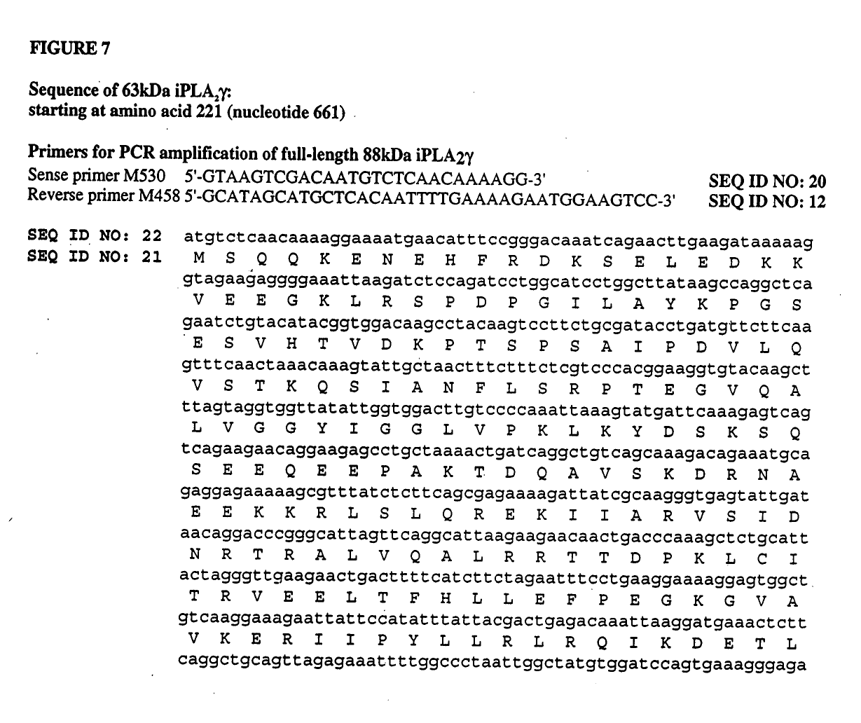

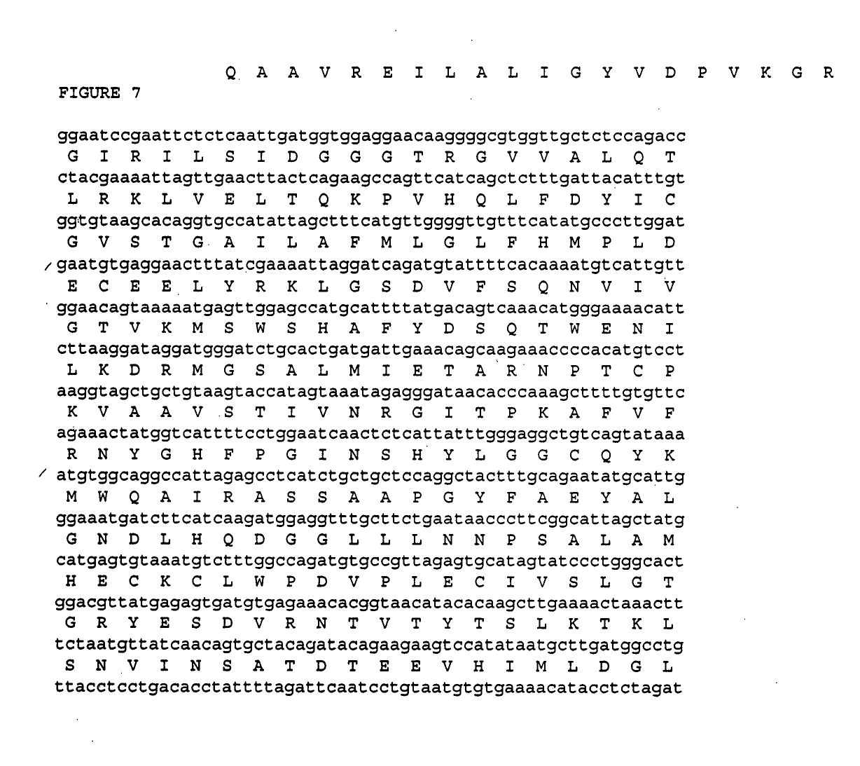

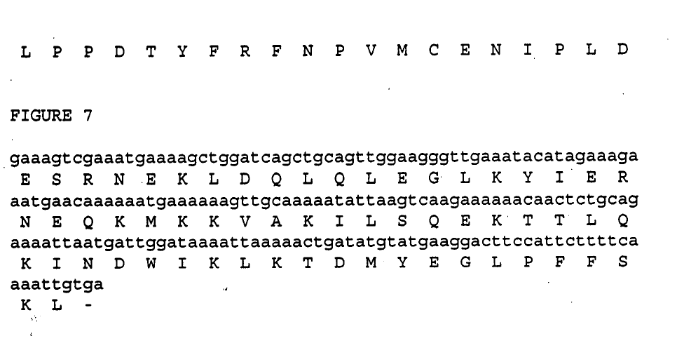

Reference to iPLA₂γ or iPLA₂γ polypeptides herein is intended to be construed to include the polypeptides corresponding to coding regions of three spliced variants identified herein. The amino acid sequences of the polypeptides are as set forth in SEQ ID NO:1 and SEQ ID NO:2. The coding portion of the cDNA of the splice variants are as set forth in SEQ ID NOS 3 and 4 (splice variants gamma 1 and gamma 2) and SEQ ID NO:5 (splice variant gamma 3) (see FIGS. 2-4). The iPLA₂γ polypeptides within the present invention are also intended to include iPLA₂γ of any origin which are substantially identical to and which are biologically equivalent to the iPLA₂γ polypeptides characterized and described herein. Such substantially identical iPLA₂γ may be native to any tissue or species and, similarly, biological activity can be characterized in any of a number of biological assay systems.

As used herein, the term “biologically equivalent” is intended to mean that the compositions of the present invention are capable of demonstrating some or all of the same sn-2-lipase activity, membrane localization and sensitivity to BEL inhibition, however, not necessarily to the same degree as the iPLA2γ isolated herein as described in the examples below.

By the phrase “substantially identical” it is meant that the degree of identity of human iPLA₂γ to the iPLA₂γ from any species is greater than that between the iPLA₂γ and any previously reported member of the iPLA₂ family. Sequence identity or percent identity is intended to mean the percentage of same residues between two sequences when the two sequences are aligned by methods well known to those skilled in the art (for example, see Higgins et al, Cabios 8:189-191, 1992; Dayhoff et al., in ATLAS OF PROTEIN SEQUENCE AND STRUCTURE, Dayhoff, Ed., NDRF, Washington, Vol. 5, suppl. 3, p. 345, 1978).

Further, polypeptides considered to be included within the term iPLA_{2γ} polypeptides also includes conservatively substituted variants of SEQ ID NO:1 or SEQ ID NO:2. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is seine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups include: valine-leucine-isoleucine, phenylalanine-tyrosine, lysine-arginine, alanine-valine, and asparagine-glutamine, glutamic acid-aspartic acid, leucine-methionine; glutamine-histidine.

In one embodiment, iPLA₂γ encompasses a class of molecule which shares homology with human iPLA_{2γ} (SEQ ID NO: 1), includes a patatin domain and ATP binding and lipase consensus sites, and is phylogenetically more closely related to iPLA₂γ sequence than either of the other members of the patatin derived proteins, iPLA₂γ or potato patatin. In this embodiment, iPLA₂γ would include mouse and rat homologs sharing 90% homology as well as other homologs from other species including C. elegans or Plasmodium falciparum which, for example, share 38-57% homology with human iPLA₂γ (SEQ ID NO: 1). In another embodiment, iPLA₂γ includes sequences from species sharing approximately 90% homology with SEQ ID NO: 1 and in particular human sequences derived from the iPLA_{2γ} gene on chromosome 7 which include at least 11 splice variants (Table 2) as well as truncated polypeptides derived from use of alternative ATG start sites. Each of these variants potentially has catalytic activity since, in each case, the lipase active site is not modified by the splicing or truncation. In this embodiment, expression of the full-length form (SEQ ID NO: 13) encoding the 88 kDa polypeptide (SEQ ID NO: 1) results in expression of truncated isoforms with catalytic activity in both a baculoviral expression system and in transgenic mice.

The iPLA₂γ polynucleotides of the present invention include those nucleic acid molecules, both DNA and RNA, which encode the iPLA_{2γ} polypeptides. Such iPLA_{2γ} polypeptides include, in particular, the coding regions of splice variants as set forth in SEQ ID NOS 3 and 4 (splice variants gamma 1 and gamma 2) and SEQ ID NO:5 (splice variant gamma 3) (see FIGS. 2-4) as well as their complements, SEQ ID NOS 7, 8 and 9, respectively. Also included are cDNA sequences which show at least 20% identity with the cDNA sequences of SEQ ID NOS 3, 4, 5, 6, 7, 8 or 9 as defined above and sequences which specifically hybridize to SEQ ID NOS 3, 4, 5, 6, 7, 8 or 9.

The phrase “by specifically hybridizing to said sequences”, is meant that a given sequence hybridizes under high stringency conditions. The stringency of hybridization is determined by a number of factors during hybridization and during the washing procedure, including temperature, ionic strength, length of time and concentration of formamide. Such factors are outlined in, for example, Sambrook et al. (Sambrook et al. Molecular Cloning: A Laboratory Manual, 2d ed. (1989) Cold Spring Harbor Laboratory Press, Cold Spring Harbor, N.Y.). High stringency conditions serve to prevent false positives, that is the hybridization and apparent detection of iPLA₂γ polynucleotide sequences when in fact an intact and functioning iPLA₂γ polynucleotide is not present.

Probes and primers which are capable of hybridizing to an iPLA₂γ sequence are also included within the present invention. Probes can be oligonucleotides of from about 25 to about 100 nucleotides in length which specifically hybridize to a target sequence. Primers within the scope of the present invention are typically short strands of DNA or RNA having a length of at least from about 6 to about 8 nucleotides, more preferably about 12 nucleotides, still more preferably about 15 nucleotides, and most preferably about 20 to about 30 nucleotides or greater in length. Such nucleic acid sequences hybridize to the target region. The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The present invention includes fragments and truncated polynucleotides which can be used in modulating the translational regulation of iPLA₂γ, and in the identification of naturally occurring repressor substances within the cell. Thus, by cloning of portions of the wild type iPLA₂γ polynucleotide into a vector one can identify those portions of the sequence involved in transcriptional or translational regulation within the cell. These polynucleotide portions of the iPLA₂γ sequence can bind to naturally occurring repressor substances in a cell and serve as binding ligands to the repressor substance. Such binding can block the decrease in iPLA₂γ polynucleotide expression action that would otherwise occur if the repressor substance were free to bind to the iPLA₂γ polynucleotide. In addition, the binding to a repressor substance by the polynucleotides can serve as a basis for identifying and isolating new compounds which prevent the binding of the repressor substance to iPLA₂γ polynucleotides. Such binding to the repressor substance produces a de-repressor action to allow an increased expression of iPLA₂γ polynucleotides.

The present invention also includes treatment of certain diseases using iPLA₂γ, protein products thereof, or a translational repressor molecule thereof. Based on its genomic characterization and biological activity, iPLA₂γ is a likely candidate for involvement in a number of disease processes. Without being bound to a particular theory, the inventors herein have discovered that iPLA_{2γ} is a likely candidate for involvement in Alzheimer's disease because its sn-2-lipase activity regulates the release of lipid second messengers. Such second messengers include arachidonic acid and its eicosanoid metabolites which are prominent lipid second messengers for inflammatory processes. (See e.g. Needleman et al., supra). It has been previously established that the neural damage underlying Alzheimer's disease ultimately results from a final common pathway involving precipitation of amyloid protein in a plaque formation that initiates lipid inflammation and produces dystrophic changes in the affected neurons. The amyloid plaques of Alzheimer's disease are a nidus for localized inflammation, and the neural dystrophic process in Alzheimer's disease is characterized by the presence of several biochemical markers of inflammation, and inflammation may be the proximal cause of synaptic loss during neural dystrophy. (See e.g. Farooqui et al., supra). Thus, the inventors herein have, in identifying and characterizing iPLA₂γ, also succeeded in identifying iPLA₂γ as a new candidate for mediating inflammatory processes involved in Alzheimer's disease. Treatment for Alzheimer's disease would thus involve any treatment that decreases the activity of iPLA_{2γ}, especially in brain regions affected by Alzheimer's disease.

Similarly, iPLA₂γ may be regarded as a candidate for mediating other inflammatory processes such as asthma.

The iPLA₂γ polypeptides of the present invention can also be used in the treatment of atherosclerotic heart disease, which represents a major cause of morbidity and mortality in the United States and other industrialized nations. In patients with myocardial infarction, the infarct size and type (i.e. transmural or subendocardial) profoundly affect realignment and remodeling of affected myocardial zones, and hence affect whether the infarct results in heart failure, or merely electrophysiologic dysfunction. Recent experimental results have shown that agents that enhance the efficiency of ATP production from the available O2 supply have a salutary effect on the size and type of myocardial infarction. These findings implicate the phenomenon known as oxygen wasting, and pose as therapeutic candidates any agents that may have a beneficial effect on the efficiency of O2 utilization by reducing oxygen wasting. In normal myocardium, most fatty acid oxidation occurs in the mitochondrial compartment, while a smaller but not insignificant portion of fatty acid oxidation, about 3-4%, occurs in peroxisomes. In the peroxisomal compartment, fatty acid oxidation occurs without efficient production of reducing equivalents but heat and resulting in oxygen wasting. During myocardial ischemia, mitochondrial oxidation of fatty acids is impaired to about 10% of normal levels. Thus, the oxygen wasted in the peroxisomal compartment gains greater significance because oxygen availability becomes rate-limiting. Therefore, reduction in oxygen wasting in the peroxisomal compartment during myocardial ischemia is likely to profoundly benefit myocyte preservation with attendant desirable effects on the clinical sequelae of ischemia or infarct. Thus, the inventors herein have, in identifying and characterizing iPLA_{2γ}, have also succeeded in identifying iPLA₂γ as a new candidate target for limiting oxygen wasting. Treatment for myocardial ischemia or myocardial infarction would thus also involve any treatment that decreases the activity of iPLA_{2γ}, especially in ischemic heart tissue.

One approach for decreasing the activity of iPLA₂γ is by the identification and administration to a living cell, a translational repressor substance which decreases translation of iPLA₂γ or administration of the nucleic acid encoding the translational repressor substance. Such repressor molecules can be identified by virtue of their binding to treanslational repressor binding sites identified herein.

Alternatively, iPLA₂γ activity can be decreased by treatment with iPLA₂γ antisense oligonucleotides. Such antisense oligonucleotides interact through base pairing with a specific complementary nucleic acid sequence involved in the expression of iPLA₂γ such that the expression of iPLA₂ is reduced. The iPLA₂γ antisense oligonucleotides preferably comprise a sequence containing from about 8 to about 100 nucleotides and more preferably the iPLA₂γ antisense oligonucleotides comprise from about 15 to about 30 nucleotides. The iPLA₂γ antisense oligonucleotides can also contain a variety of modifications that confer resistance to nucleolytic degradation such as, for example, modified internucleotide lineage's (Uhlmann and Peyman, Chemical Reviews 90:543-548 1990; Schneider and Banner, Tetrahedron Lett. 31:335, 1990), modified nucleic acid bases and/or sugars and the like.

Agents that regulate or control peroxisomal β-oxidation are also potentially useful in controlling the related diseases of obesity, hyperlipidemia and diabetes, which represent major health problems in the United States and other industrialized nations. Obesity and hyperlipidemia are primary features of the metabolic syndrome, which identifies those at high risk for developing insulin-resistant (Type II) diabetes. Obesity and high serum lipid levels also predispose individuals to developing atherosclerosis and hypertension. Accordingly, agents that decrease body weight, serum lipid levels or the insulin-resistance characterizing Type II diabetes, are avidly sought. One type of such agents operates by increasing caloric expenditure or fatty acid α and β-oxidation, and significant reductions in body weight can be achieved with only small increases (about 2-3%) in caloric expenditure. Since fatty acid β-oxidation in peroxisomes wastes oxygen, producing only heat, and no energy in the form of NADH or ATP, an agent that thus increases caloric expenditure by increasing peroxisomal fatty acid β-oxidation, directly or indirectly, has potentially major beneficial effects in the treatment of obesity, hyperlipidemia, diabetes, atherosclerosis and hypertension. Thus, the inventors herein have, in identifying and characterizing iPLA₂γ, also succeeded in identifying iPLA₂γ as a new candidate target for increasing caloric expenditure by increasing peroxisomal fatty acid γ and β-oxidation. Accordingly, treatment for the related diseases of diabetes, obesity, and hyperlipidemia would thus also involve any treatment that modulates the activity of iPLA₂γ, especially in Type II diabetic or obese individuals.

The present invention, therefore, also includes therapeutic or pharmaceutical compositions comprising iPLA₂γ polypeptides and polynucleotides as well as a blocker of iPLA₂γ translational repressor molecule, in an effective amount for treating patients with inappropriately low levels of iPLA₂γ. Such diseases include but are not limited to Type II diabetes, obesity and hyperlipidemia. The treatment can comprise administering a therapeutically effective amount of iPLA₂γ polynucleotide or iPLA₂γ protein or an effective amount of blocker of iPLA₂γ translational repression.

The therapeutic or pharmaceutical compositions of the present invention can be administered by any suitable route known in the art including for example intravenous, subcutaneous, intramuscular, transdennal, intrathecal or intracerebral. Administration can be either rapid as by injection or over a period of time as by slow infusion or administration of slow release formulation.

iPLA₂γ can also be linked or conjugated with agents that provide desirable efficiencies pharmaceutical or pharmacodynamic properties. For example, iPLA₂γ can be coupled to any substance known in the art to promote penetration or transport across the blood-brain barrier such as an antibody to the transferrin receptor, and administered by intravenous injection (See, for example, Friden et al., Science 259:373-377, 1993). Furthermore, iPLA₂γ can be stably linked to a polymer such as polyethylene glycol to obtain desirable properties of solubility, stability, half-life and other pharmaceutically advantageous properties. (See, for example Davis et al. Enzyme Eng 4:169-73, 1978; Burnham, Am J Hosp Pharm 51:210-218, 1994).

In an aspect, the compositions are employed in the form of pharmaceutical preparations. Such preparations are made in a manner well known in the pharmaceutical art. In an aspect, one preparation utilizes a vehicle of physiological saline solution or, but it is contemplated that other pharmaceutically acceptable carriers such as physiological concentrations of other non-toxic salts, five percent aqueous glucose solution, sterile water may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. Calcium-independent phospholipase A₂γ can also be incorporated into a solid or semi-solid biologically compatible matrix that can be implanted into tissues requiring treatment.

The carrier can also contain other pharmaceutically-acceptable excipients and additives for modifying or maintaining pH, osmolarity, viscosity, clarity, color, sterility, stability, rate of dissolution, or odor of the formulation. Similarly, the carrier may contain still other pharmaceutically acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion. Dose administration can be repeated depending upon the pharmacokinetic parameters of the dosage formulation and the route of administration used.

It is also contemplated that certain formulations containing iPLA₂γ are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl-and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In a number of circumstances it would be desirable to determine the levels of containing iPLA₂γ in a patient sample. The term “detection” as used herein in the context of detecting the presence of iPLA₂γ in a patient is intended to include determining the amount of iPLA₂γ or the ability to express an amount of iPLA₂γ in a patient, the distinguishing of iPLA₂γ from other phospholipases A₂, the estimation of prognosis in terms of probable outcome of a degenerative disease and prospect for recovery, the monitoring of iPLA₂γ levels over a period of time as a measure of status of the condition, and the monitoring of iPLA₂γ levels for determining a preferred therapeutic regimen for the patient.

To detect the presence of iPLA₂γ in a patient, a sample is obtained from the patient. The sample can be a tissue biopsy sample or a sample of blood, plasma, serum, CSF (cerebral spinal fluid) or the like. Samples for detecting iPLA₂γ can be taken from any of these tissues. To detect the presence of iPLA₂γ, an enzymatic analysis or Western blot analysis can be performed.

The term “probe” as used herein refers to a structure comprised of a polynucleotide which forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20, more preferably a minimum of about 25, still more preferably a minimum of about 30, yet still more preferably a minimum of about 40, and most preferably a minimum of about 50. Calcium-independent A₂γ gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence, in situ hybridization, and single-strand conformation polymorphism with PCR amplification.

Hybridization is typically carried out at 25-45° C., more preferably at 32-40° C., and more preferably at 37-38° C. The time required for hybridization is from about 0.25 to about 96 hours, more preferably from about 1 to about 72 hours, and most preferably from about 4 to about 24 hours.

The PCR method for labeling probes or amplifying target sequences uses primers that flank or lie within the target, here iPLA₂γ gene. The PCR method is well known in the art. Briefly, PCR is performed using two oligonucleotide primers which are capable of hybridizing to the nucleic acid sequences flanking a target sequence that lies within a iPLA₂γ gene and amplifying the target sequence. The terms “oligonucleotide primer” as used herein refers to a short strand of DNA or RNA ranging in length from about 8 to about 50 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, the method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified. Preferably, primers are at least 10 nucleotides long, more preferably at least 15 nucleotides long, still more preferably at least 20 nucleotides long, still more preferably at least 25 nucleotides long, still more preferably at least 30 nucleotides long, still more preferably at least 40 nucleotides long, and most preferably at least 50 nucleotides long. After PCR amplification, the DNA sequence comprising iPLA₂γ or a fragment thereof is then directly sequenced and analyzed.

Exemplary embodiments of the invention are described in the following examples. Other alternative embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

The following methods were used in the examples below. Specific materials used in the work reported herein were as described herein, but those skilled in the art will recognize that many suitable alternative materials are readily available from commercial suppliers. [α-³²P]dCTP (6000 Ci/mmol) and ECL detection reagents were purchased from Amersham Pharmacia Biotech. A human heart cDNA library was purchased from Stratagene, Inc. Human heart Marathon cDNA, QuickClone human skeletal cDNA, and human MTN multiple tissue Northern blots were purchased from CLONTECH. For PCR, a Perkin-Elmer Thermocycler was used, and all PCR reagents were purchased from PE Biosystems. The pGEM-T vector and TnT Quick Coupled Transcription/Translation System were purchased from Promega. Vector pcDNA1.1 was purchased from Invitrogen. Culture media, Cellfectin and LipofectAMINE reagents for transfection of baculovirus vectors, and competent DH110Bac Escherichia coli cells were purchased from Life Technologies, Inc., and used according to the manufacturer's protocol. QIAfilter plasmid kits and QIAquick gel extraction kits were obtained from Qiagen, Inc. Keyhole limpet hemocyanin was obtained from Pierce. L-α-1-dipalmitoyl-2-[1-¹⁴C] phosphatidylcholine, L-α-1-palmitoyl-21-[-¹⁴C] linoleoyl phosphatidylcholine, and L-α-1-palmitoyl-2-[1-¹⁴C] linoleoyl phosphatidylcholine, and L-α-1-palmitoyl-2-[1-αC] arachidonyl phosphatidyleholine were purchased from NEN Life Science Products. 1-O-(Z)-hexadec-1′-enyl-2-[9,10-3H]octadec-9′-enoyl-sn-glycero-3-phosphocholine was synthesized and purified as described in Han et al., Anal. Biochem. 200:119-24, 1992, which is herein incorporated by reference. The luciferase assay kit used was that of Promega. BEL was obtained from Sigma. Any other reagents were also obtained primarily from Sigma. Searches of EMBL and NCBI databases were performed using the Basic Local Alignment Search Tool (BLAST) (NCBI). Alignments of all sequences were performed using the MultiAlign computer program, as described in Corpet, F., Nucleic Acids Res. 16:10881-90, 1988, which is herein incorporated by reference.

Expression of iPLA₂γ in an intact eukaryotic system was evaluated using cultured Spodoptera frugiperda (Sf9) cells. Sf9 cells have been widely used to express recombinant proteins as a host cell in the baculovirus expression system. (See e.g. Liu et al., J. Immunol. 156:3292-300, 1996). It is believed that the translational elements of Sf9 cells exhibit a high degree of homology with mammalian translational elements. (See e.g. Sakahira et al., .J. Bio. Chem. 274:15740-4, 1999; Kimball et al., J. Biol. Chem. 273:12841-5, 1998; Gu et al., Biochim. Biophys. Acta 1399:1-9, 1998). Sf9 cells were grown and infected with wild-type or recombinant baculovirus containing human phospholipase A2γ cDNA as described in detail in Wolf, M. J. and Gross, R. W., J. Bio. Chem. 27 1:30879-85, 1996, which is herein incorporated by reference. Culture of Sf9 cells involved, in brief, placing Sf9 cells in growth medium in 100-ml spinner flasks equipped with a magnetic spinner. The rotation speed of the magnetic spinner was set at about 80 rpm, which is believed to be especially suitable for optimizing growth of Sf9 cells. The growth medium included Grace's medium supplemented with Yeastolate, Lactalbumin, 1% Antimycotic (all from Sigma), 10% fetal bovine serum and 1% Plutonic F-68 surfactant. Sf9 cells at a concentration of 1×106 cells/ml were prepared in 50 ml of the growth medium and incubated at 27° C. for one (1) hour prior to infection with either wild-type baculovirus or recombinant baculovirus containing human phospholipase A₂γ cDNA. After 48 hours, cells were repelleted by centrifugation, resuspended in ice-cold phosphate-buffered saline, and repelleted. All subsequent operation were performed at 4° C. The supernatant was decanted, and the cell pellet was re-suspended in 5 ml homogenization buffer (25 mM imiazole, pH 8.0, 1 mM EGTA, 1 mM dithiothreitol, 0.34 M sucrose, 20 MM transepoxysuccinyl-L-leucylamido-(4-guanidino) butane, and 2/ml leupeptin. Cells were lysed at 0° C. by sonication (twenty (20) one-second bursts utilizing a Vibra-cell sonicator at a 30% output) and centrifuged at 100,000×g for one (1) hour. The supernatant containing predominantly cytosol was saved, and the membrane pellet was washed with homogenization buffer and resuspended using a Teflon homogenizer in 6 ml of homogenization buffer. After a brief sonication (ten (10) one-second bursts), the mixture was subjected to recentrifugation at 100,000×g for one (1) hour. After removal of the supernatant, the membrane pellet was resuspended in 1 ml homogenization buffer using a Teflon homogenizer and subsequently sonicated at 0° C. for five (5) one-second bursts.

For PCR, primer design and selection of appropriate restriction sites for constructing vectors proceeded according to the following. The free Internet software program Amplify 1.2 was used to design nucleotides containing appropriate terminal restriction sites. In general, oligonucleotides are at least 20 nucleotides long, with approximately 50% GC/AT ratio, and do not have a T at the 3′ end. The Amplify program predicts the efficiency of nucleotide priming, the presence of primer dimer pairing, if any, and the predicted sizes and approximate amounts of products obtained. The iPLA₂γ used for designing primers has been deposited in the GenBank® database (accession number AF263613).

For PCR amplification of iPLA₂γ, the reagents (10×PCR buffer, Mg buffer, dNTPs, and Amplitaq) were obtained from PE Biosystems. Plugged pipette tips were used in all PCR procedures to minimize cross-contamination. PCR amplification occurred in the Perkin Elmer model 460 thermocycler. PCR reactions (50 l) were set up as follows:

| | |
|---|---|
| 10x PCR buffer | 5 μl |
| Mg | 3 μl |
| 10 mM dNTO mix | 1 μl each |
| Sense primer | 0.5 μl of 0.08 mM stock |
| Antisense primer | 0.5 μl of 0.08 mM stock |
| DNA template 1 | 1 μl of miniprep (from a 50 μl miniprep |
| | stock) |
| Sterile millipore water | 35.75 μl |

The thermocycler was programmed to heat the above reaction mixture for five minutes at 94° C. followed by two minutes at 80° C. during which 0.25 μl Amplitaq and one drop of oil were added prior to initiation of the cycling program. For PCR analysis, a 30-cycle program was employed, with steps at 53° C. for 30 seconds, 72° C. for two minutes, and 94° C. for 30 seconds per cycle. IPLA2γ was amplified utilizing oligonucleotides that flanked the predicted 5′- and 3′-coding region, M444 (5′-TTTTGTCGACATGTCTATTAATCTGACTGTAGATA-3′)(SEQ ID NO: 11) and M449 (5′-GCATACTCGAGTCACAATTTTGAAAAGAATGGAAGTCC-3′) (SEQ ID NO:23), respectively to engineer appropriate restriction sites onto iPLA2γ for subsequent cloning into SalI/SphI restriction sites of a pFASTBAC vector (Life Technologies, Inc.). PCR screening was performed using human skeletal muscle cDNA (0.5 ng), human heart Marathon cDNA (0.5 ng) and a human heart cDNA library (approximately 1×109 plaque-forming units) as templates. To directly compare differences between the sequences reported herein and previously reported sequences, PCR amplification of sequence previously reported in a BAC genomic clone (Research Genetics) was used as a template, and PCR was performed with primers M452 (5′-GTACATACGGTGGACAAGCCTA-3′)(SEQ ID NO:24) and M446 (5′-CATTCCTCTCCCTTTCACTGGATCCACATAGCC-3′)(SEQ ID NO:25).

PCR products were resolved by 1% agarose gel electrophoresis. Candidate bands visualized with ethidium bromide staining were extracted from the agarose gel using the QIAquick Gel extraction kit from Qiagen. The extracted DNA and cloning vector (PFASTBAC) were restriction digested with appropriate restriction enzymes (SaII/SphI for the above primers), resolved on a 1% agarose gel, extracted with the QlAquick Gel extraction kit, and then ligated. In a typical ligation 2 to 10 molar excess restriction digested PCR product was ligated overnight at 16° C. to restriction digested vector in a 20 μl reaction using T4 DNA ligase (Promega). For transformation, Stratagene XL2-Blue MRF ultracompetent cells (#200151) were used according to the manufacturer's protocol. Following bacterial transformation and growth of transformants, plasmids were purified using a QIAfilter plasmid kit from Qiagen, and subjected to automated sequence analysis using either an ABI 373S or 377XL automated DNA sequencer (PE Biosystems).

Bacmids were prepared from the pFASTBAC plasmid constructs using the Bac-to-Bac Baculovirus Expression System protocol from Life Technologies, Inc. In brief, pFASTBAC plasmid constructs were used to transform DH1OBac competent cells resulting in a Tn7 site-specific transposition to produce a composite bacmid containing the cloned sequence of interest. This bacmid was used in Cellfectin-mediated transfection of Sf9 cells in a 35 mm plates for 72 hours resulting in production of infectious recombinant baculovirus in the Sf9 cells. Recombinant baculovirus (1 ml) obtained was used to infect 50 ml of Sf9 cells in a 100 ml spinner for an additional 72 hours to provide a stock of amplified virus.

To prepare truncated iPLA2γ constructs in pFASTBAC, PCR primers containing appropriate restriction sites for cloning were used. For example, the original full-length construct was used as a template in PCR with a sense primer annealing at nt 650-676 (SEQ ID NO:20) paired with reverse primer M458 (SEQ ID NO: 12) to create a truncated iPLA2γ (63 kDapF) with a methionine start site corresponding to amino acid 221 in the full-length protein, expression of this construct in the baculovirus system resulted in a 63 kDa recombinant protein. A second construct was created with a start site corresponding to methionine 122 to produce a 74 kDa recombinant protein (SEQ ID NO. 18) using PCR primers corresponding to SEQ ID NOS 17 and 12. Similarly, PCR primers M534 (SEQ ID NO: 14) and M458 (SEQ ID NO: 12) were used to prepare a PCR product containing the 77 kD coding sequence (SEQ ID NO: 16) for cloning into pFASTBac vector and subsequent expression of the 77 kDa polypeptide (SEQ ID NO: 15 ) in the baculoviral expression system. Segments of iPLA2γ 5′ sequence ranging between 15-663 nt were inserted 5′ of the insert sequence in 63 kDapF to identify potential sequences involved in translational regulation. These sequences were ligated as in frame 5′ coding sequence or as 5′ noncoding sequence to the 63 kDapF construct. For recombinant protein produced in the Sf9 system as described herein, enzymatic activity was determined using the enzyme assay as described below in Example 6, and mass of each of these constructs was determined using Western analysis as described herein. Additionally, full-length truncated iPLA₂γ and truncated iPLA₂γ constructs were inserted 5′ of full-length luciferase coding sequence which was cloned into pFASTBAC via NotI/XbaI restriction sites. Luciferase activity of recombinant protein produced in the Sf9 system was then measured using the Luciferase Assay System of Promega, as described in detail in Example 8 below. (Wood, K. V., (1991) In: Bioluminescence and Chemiluminescence: Current Status, Stanley, P. and Kricka, L. eds., John Wiley and Sons, Chichester, N.Y., 543; and Luciferase Assay System Technical Bulletintus, #TB 101, Promega Corporation, 1996, which are herein incorporated by reference).

Furthermore, each of the truncated forms can be cloned into vectors (such as alpha MHC vector) for generation of transgenic mice expressing each truncated form as a recombinant protein. Constructs can also be modified by addition of appropriate tags (such as a 6×His (SEQ ID NO: 54)) for generation of tagged recombinant protein facilitating their purification and characterization. The procedure for generation of transgenic mice is described in Example 11. Expression of full-length and truncated forms in transgenic mice will facilitate studies of the functions of iPLA₂γ in whole animals (as opposed to in vitro or in tissue culture cells) and will help elucidate the role of iPLA₂γ in normal and abnormal physiology (e.g. lipid homeostasis, myocardial disorders, and diabetes).

The unique ability of antibodies to recognize and specifically bind to target proteins provides an approach for treating an over expression of the protein. Thus, another aspect of the present invention provides for a method for preventing or treating diseases involving over expression of the iPLA₂γ protein by treatment of a patient with specific antibodies to the iPLA₂γ protein. Specific antibodies, either polyclonal or monoclonal, to iPLA₂γ protein can be produced by any suitable method known in the art. For example, the iPLA₂γ protein, or an immunologically active fragment thereof, or an anti-idiotypic antibody, or fragment thereof can be administered to an animal to elicit the production of antibodies capable of recognizing and binding to the iPLA₂γ protein. Such antibodies can be from any class of antibodies including, but not limited to IgG, IgA, IgM, IgD, and IgE or in the case of avian species, IgY and from any subclass of antibodies. Polyclonal antibodies can be prepared by immunizing rabbits or other animals by injecting antigen followed by subsequent boosts at appropriate intervals. The animals are bled and sera assayed against purified iPLA₂γ protein usually by ELISA or by bioassay based upon the ability to block the activity of iPLA₂γ protein in cells. When using avian species, e.g. chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, METHODS IN ENZYMOLOGY: IMMUNOCHEMICAL TECHNIQUES 73:1-46, Langone and Banatis eds., Academic Press, 1981 which are incorporated by reference). The hybridoma cells so formed are then cloned by limiting dilution methods and supemates assayed for antibody production by ELISA, RIA or bioassay.

More specifically, to generate and purify the anti-iPLA₂γ peptide polyclonal antibodies used in the work described herein, New Zealand white rabbits were immunized with the iPLA₂γ synthetic peptide CENIPLDESRNEKLDQ (SEQ ID NO:26). This peptide (G3, 4 mg), dissolved in minimal volume of dimethylsulfoxide, was conjugated to maleimide-activated keyhole limpet hemocyanin (KILH, 2 mg) in 200 ld of 83 mM sodium phosphate, 0.9 M NaCl , and 0.1 mM EDTA at 22° C. for two hours. After extensive dialysis against 83 mM sodium phosphate containing 0.9 mM NaCl, a 1:1 emulsion was made with the peptide-KLH conjugate and Freund's complete adjuvant. This emulsion was then injected subcutaneously into two anaesthetized New Zealand white rabbits. Two booster injections of a 1:1 emulsion of the peptide-KLH conjugate and Freund's incomplete adjuvant were given two and four weeks after the initial immunization. Serum was then collected for purification of the anti iPLA2γ antibodies.

A peptide affinity column was generated by coupling the iPLA_{2γ} synthetic peptide to a thiopropyl-Sepharose (Amersham-Pharmacia) resin in the presence of 100 mM sodium citrate, pH 4.5. The resin-peptide suspension was mixed by inversion overnight at 22° C. Coupling efficiency was monitored by the displacement of the 2-pyridyl sulfide groups as 2-thippyridone through spectrophotometric monitoring at 343 mn. Before chromatography, the resin-peptide suspension was equilibrated with 10 mM Tris-HCl, pH 7.5 in an Econo-pac column (BioRad) by gravity flow. Serum containing the anti the iPLA2γ antibodies was diluted 1:10 (v/v) with 10 mM Tris-HCl, pH 7.5 and then applied to the peptide affinity column by gravity flow. The diluted serum was then re-applied twice to the column to assure binding of the antibody to the column. After washing the column extensively with 10 mM Tris-Hcl, pH 7.5 containing 500 mM NaCl, bound anti-iPLA2γ antibody was eluted from the column with 100 mM glycine, pH 2.5 and the immediately neutralized with 100 mM Tris-Hcl, pH 9.0. The antibody was then concentrated using Centricon-10 (Americon) centrifugal filter devices and stored at 40° C. until use.

### EXAMPLE 1

This example illustrates the approach used in identifying the full-length cDNA encoding phospholipase A₂γ.

Inspection of a sequence encoding a putative 40 kDa phospholipase reported by the Human Genome Sequencing Project (BAC clone RG054D04; GenBank® accession no. AC005058) demonstrated that the sequence did not begin with an initiator methionine codon. Thus, a TBLASTN data base search was undertaken to find expressed sequence tags (EST's) that might align with the 5′-end of the putative phospholipase sequence.

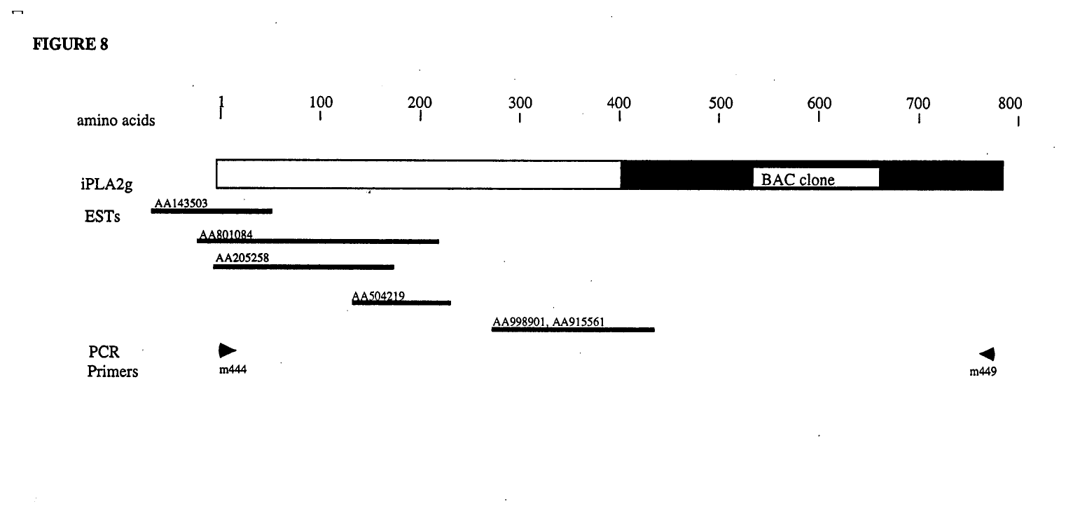

Six expressed sequence tags (EST's) identified in the TBLASTN search were mapped on the basis of their overlapping the 40 kDa cDNA sequence or on the basis of their binding in close spatial proximity to the putative phospholipase sequence. FIG. 8 is a schematic illustration showing this mapping. Amino acid numbering starting from the most 5′ potential translation site is indicated by the scale at top, with a schematic representation of the previously identified putative 40-kDa phospholipase shown shaded below. The EST sequences that were used to identify a potential N-terminal initiator methionine and to map the full-length sequence of a novel phospholipase, calcium-independent phospholipase A₂γ (iPLA_{2γ}), are indicated by solid bars. The accession number for each EST sequence is indicated above each solid bar. The arrows at bottom indicate the position and orientation of PCR primers M444 (5′-end) and M449 (3′-end) used to amplify a full-length 2.4-kb coding sequence for iPLA2γ from human heart cDNA.

Specifically, EST clones vz36b01.ri Soares 2NbMT Mus musculus cDNA IMAGE: 1328521 (accession no. AA915661) and Rattus norvegicus cDNA UI-R-C0-hp-c-06-0-UI (accession no. AA998901) were found to overlap with the BAC clone sequence, thereby adding an additional 360 nt sequence to the previously known 5′ end of the putative phospholipase sequence. Four other EST clones found in the GenBank® database (Stratagene Homo sapiens colon cDNA clone IMAGE:5 88479, accession no. AA143503; Stratagene H. sapiens cDNA clone IMAGE:647744, accession no. AA205528; normalized rat ovary, Bento soares Rattus sp. cDNA clone ROVAA46, accession no. AA801084; and NCI_CGAP_GCB1 H. sapiens cDNA clone IMAGE:825005, accession no. AA504219) were found to be in close spatial proximity to the putative phospholipase sequence from the BAC clone. However, when aligned with the BAC clone sequence, the 3′-end of the EST AA504219 sequence and the 5′-end of EST AA998901 are separated by a 150-nt gap, as shown in FIG. 8. Moreover, when the EST AA998901 sequence was back-translated through the gap and into EST AA504219, a continuous reading frame resulted. Thus, by overlapping known EST sequences and back-translating through the 150-nt gap, the inventors have succeeded in extending the putative phospholipase sequence approximately 1.2 kb upstream from the predicted GenBank® protein product.

The furthest upstream ATG codon that remained in frame with the newly identified full-length coding sequence was located 1210 nt upstream from the previously reported BAC clone sequence. Translation of the reported gene sequence further 5′ from nt 122761 in BAC clone RGO54DO4 results in stop codons in all three reading frames.

Based upon the predicted sequence obtained from this assembly of sequences in the database, primers were constructed to obtain and sequence the putative full-length phospholipase. For PCR, primer design and selection of appropriate restriction sites for constructing vectors proceeded according to the following. The free software program Amplify 1.2 was used to design nucleotides containing appropriate terminal restriction sites. In general, oligonucleotides are at least 20 nucleotides long, with approximately 50% GC/AT ratio, and do not have a T at the 3′ end. The Amplify program predicts the efficiency of nucleotide priming, the presence of primer dimer pairing, if any, and the predicted sizes and approximate amounts of products obtained. The iPLA₂ used for designing primers has been deposited in the GenBankS database (accession number AF263613).

PCR analysis used primers corresponding to the most 5′ candidate initiator methionine and the known 3′ stop codon (nt 79,673) in the gene sequence.

For typical PCR analysis, a 30-cycle program was employed with steps at 53° C. for 30 seconds, 72° C. for two minutes, and 94° C. for 30 seconds per cycle. iPLA₂γ was amplified utilizing oligonucleotides that flanked the predicted 5′-and 3′-coding region, M444 (5′-TTTGTCGACATGTCTATTAATCTGACTGTAGATA-3′) SEQ ID NO:11 and M449 (5′-GCATACTCGAGTCACAATTTTGAAAAGAATGGAAGTCC-3′) (SEQ ID NO: 23), respectively to engineer appropriate restriction sites onto iPLA2γ for subsequent cloning onto SaII/SphI restoration sites of a pFASTBAC vector (Life Technologies, Inc.).

PCR screening was performed using human skeletal muscle eDNA (0.5 ng), human heart Marathon cDNA (0.5 ng) and a human heart cDNA library (approximately 1×109 plaque-forming units) as templates. To directly compare differences between the sequences reported herein and previously reported sequences, PCR amplification of sequence previously reported in BAC genomic clone RGO54 D04 (Research Genetics) was used as a template, and PCR was performed with primers M452 (5′-GTACATACGGTGGACAAGCCTA-3′)(SEQ ID NO:24) and M446 (5′-CATTCCTCTCCCTTTCACTGGATCCACATAGCC-3′)(SEQ ID NO:25).

PCR products were resolved by 1% agarose gel electrophoresis. Candidate bands were extracted from the agarose gel using the QIAquick Gel extraction kit from Qiagen followed by blunt end ligation into the pGEM-T-Vvector (Promega) by standard procedures. (Sambrook et al., Molecular Cloning: A Laboratory Manual, 2nd ed., Cold Spring Harbor Laboratory, Cold Spring Harbor, N.Y., 1989, which is incorporated by reference). Following bacterial transformation and growth of transformants, plasmids were purified using a QIAfilter plasmid kit from CLONTECH, and subjected to automated sequence analysis using either an ABI 373S or 377XL automated DNA sequencer (PE Biosystems).

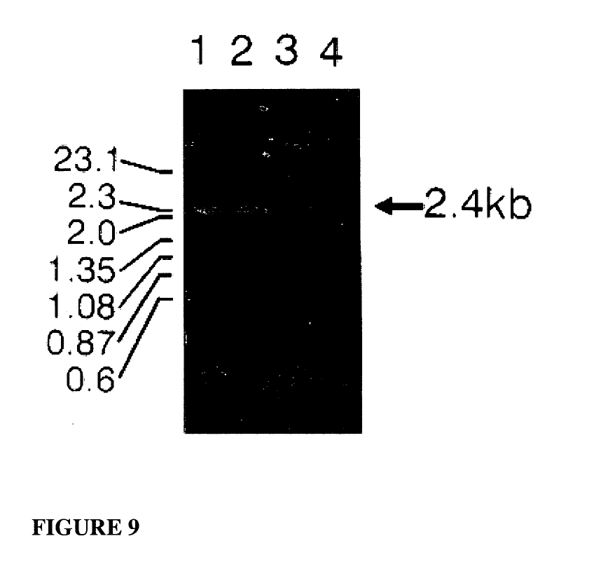

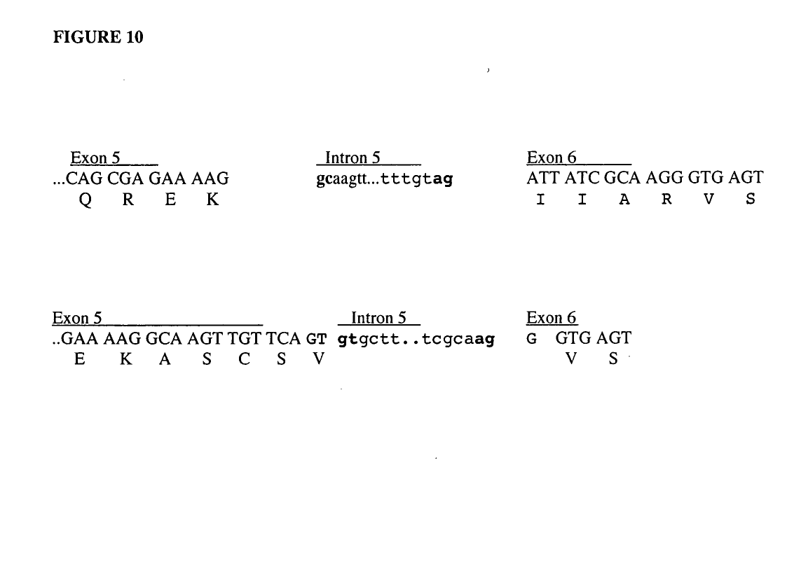

FIG. 9 shows the results of PCR amplification of the novel human iPLA2 sequence, iPLA₂γ. Specifically, PCR was performed using human heart cDNA (0.5 ng; lane 1 in FIG. 9), a cDNA library prepared from human heart (lane 2), human skeletal muscle (lane 3), and a blank control (lane 4), as templates as described in detail above. PCR primer M444 was positioned at the 5′-end, and primer M449 positioned at the 3′-end of the iPLA₂γ coding sequence, in 30 cycles of amplification at 53° C. for 30 seconds, 72° C. for 2 minutes, and 94° C. for 30 seconds. PCR products were analyzed on a 1% agarose gel and visualized using ethidium staining. Molecular size markers are shown at left in kb. PCR analysis gave rise to a single band, which was 2.4 kb in length, as indicated by the arrow.

### EXAMPLE 2

This example illustrates the determination and characterization of the full length amino acid sequence of iPLA_{2γ}, based on the PCR product described in Example 1, FIG. 9.

First, the PCR product was cloned and the sequence analyzed. More specifically, the PCR product was subeloned into pGEM-T Vector and sequenced in both directions. Based on amino acid residue I being the initiator methionine, the message encoded a 782-amino acid polypeptide with a calculated molecular weight of 88,476.9. Contained within the sequence were an ATP binding motif at amino acid residues 449-45, and a lipase consensus sequence at amino acid residues 481-485, as well as multiple cAMP phosphorylation sites, PKC phosphorylation sites, CK2 phosphorylation sites, and a microbody C-terminal targeting sequence as determined by a Prosite pattern search (Hoflnann et al., Nucleic Acids Res. 27: 215-19, 1999; Bucher, P. and Bairoch, A. ISMB-94 Proceedings: 2nd International Conference on Intelligent Systems for Molecular Biology, Menlo Park, Calif., pp. 53-61, AAAI Press, Menlo Park, Calif., 1994, which are incorporated herein by reference).

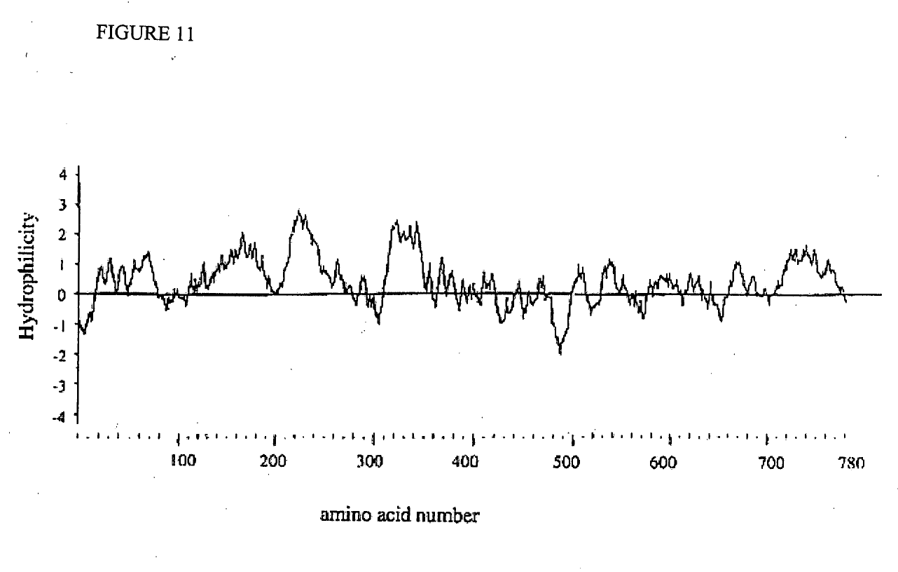

To further analyze the deduced iPLA₂γ amino acid sequence, a Kyte-Doolittle hydrophobicity analysis was performed on the sequence. (Kyte, J. and Doolittle, R. F., J. Mol. Biol. 157:105-132, 1982, which is herein incorporated by reference). FIG. 11 shows a hydropathy plot of the deduced amino acid sequence of human iPLA₂γ. A window size of 17 amino acids was used. Negative values on the y-axis represent increasing hydrophobicity. Hydrophobicity analysis thus revealed that the putative iPLA₂γ had two major hydrophobic domains, a first at the extreme putative N-terminus and a second centered near the lipase consensus sequence, at Ser483.

To determine whether the iPLA₂γ PCR product represented the true 5′-end of the coding sequence, and to locate additional message sequence 5′ of nt 122,761, 5′-RACE was performed using a reverse primer (M460) near the 5′-end of the 2.4 kb phospholipase A_{2γ}PCR product, and a sense primer (API) to the adapter sequence flanking the cDNA template.

More specifically, for 5′-RACE, a 45-cycle program with steps at 58° C. for 30 seconds, 72° C. for 2 minutes, and 94° C. for 30 seconds per cycle was employed. Human heart Marathon-ready cDNA was used as a template (0.5 ng) and primer Apl (CLONTECH) was paired with M460 (5′-GAAAACCTCTTTGTAGACTGATGTGGCTTATCCTCCAG3′)(SEQ ID NO:27) to amplify products. Products were analyzed by electrophoresis utilizing a 1% agarose gel and visualized by ethidium bromide staining. PCR products were excised from the gel, purified with a QlAquick gel extraction kit, and subdloned into pGEM-T vector (Promega) for sequencing and alignment with the iPLA₂γ sequence.

The full-length human iPLA₂γ message sequence as determined using 5′-RACE includes a putative transcription initiation site, 5′-untranslated region, coding sequence, and 3′-untranslated region that together form a 3.4 kb mature message. Thus, this maneuver extended the 5′ sequence from the putative initiator methionine residue an additional 225 base pairs upstream. Because the 225-base pair sequence contained stop codons in all three reading frames, it is believed that no additional coding regions are present in the 225-base pair sequence. The most 5′ ATG site that is in frame with the calcium-independent phospholipase A₂γ is located at nt 226. Additional in-frame ATG sites are located at nt 526, 589 and 886 (based on SEQ ID NO:6 numbering). Initiation of polypeptide synthesis at these potential methionine start sites would result in polypeptides of 77, 74 and 63 kDa, respectively. At the 3′-end of the gene, a signal site for 3′ poly(A) processing (AATAAA) was identified 757 nt 3′ of the TGA stop signal. The actual cleavage site for poly(A) addition usually occurs 10-30 nt 3′ of the poly(A) signal, and at this location, a CA is believed to be the preferred sequence immediately 5′ to the cleavage site. Additionally, GU-rich or U-rich elements are also typically found downstream of the poly(A) site. Since a CA dinucleotide occurs 35 nt 3′ of the poly(A) signal sequence in iPLA₂γ, and highly U-rich sequences occur at nucleotides 3339-3343 and at 3373-3392, the inventors have deduced that the likely point of poly(A) addition occurs following nucleotide 3372. Accordingly, these results identify a putative transcription initiation site, 5′-untranslated region, coding sequence, and 3′-untranslated region that together form a 3.4 kb mature message for iPLA₂γ.

To determine if the 3.4 kb message was the full-length or nearly full-length message, or if additional, as yet unidentified regions of the gene were transcribed to potentially serve a promoter function, Northern blot analysis of human iPLA₂γ mRNA was performed on multiple tissues.

For Northern blot analysis, full-length of iPLA₂γ amplified by PCR was prepared for use as a probe by radiolabeling with [³²P]dCTP for one (1) hour at 37° C. in the presence of Ready-to-Go labeling beads (Amersham Pharmacia) according to the instructions provided by the manufacturer. The radiolabeled probe was purified by gel filtration employing a 1-ml Sepharose G-25 spin column. For Northern analysis, and MTN blot (CLONTECH) containing 2 μg of poly(A)+ RNA/lane from human brain, heart, liver, lung, pancreas and placental tissue was prehybridized at 68° C. for 30 minutes in hybridization buffer, hybridized for one hour at 68° C. with 6 radiolabeled iPLA₂(2×106 cpm/ml), and washed in 2×SSC and 0.1% SDS twice for 30 minutes, followed by two washes with 0.1×SSC and 0.1% SDS for 40 minutes each at 50° C. as per the manufacturer's instructions. Hybridized sequences were identified by autoradiography for 16 hours.

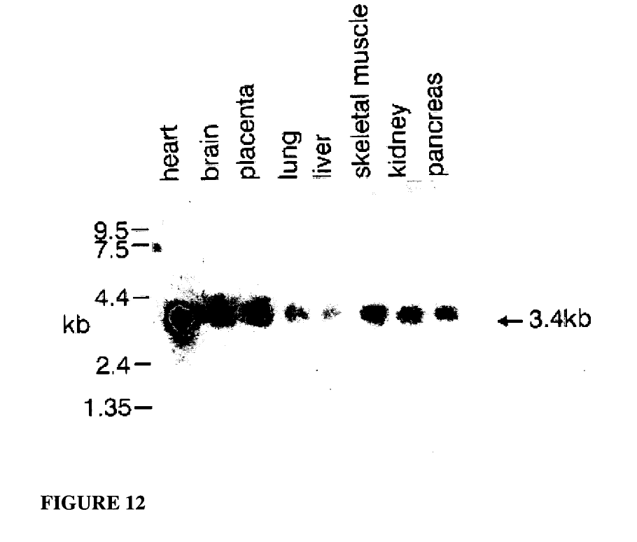

FIG. 12 shows the results of a Northern blot analysis of human iPLA₂γ mRNA. The Northern blot was human mRNA obtained from the multiple tissues, and the tissue distribution of iPLA₂γ mRNA thus examined by hybridization of the ³²P-labeled full-length iPLA₂γ with approximately 2 μg of human poly(A)+ mRNA, followed by autoradiography, as described above. Lane 1 shows the results from heart, lane 2 from brain, lane 3 from placenta, lane 4 lung, lane 5 from liver, lane 6 from skeletal muscle, lane 7 from kidney, and lane 8 from pancreas. The positions of RNA size markers are shown. Relative size in kb is indicated at left based on the mobility of the RNA standard ladder.

Our northern blotting of human heart, brain, placenta, lung, liver, skeletal muscle, kidney and pancreas MRNA demonstrated that each of these tissues possessed a 3.4 kb message that tightly bound to the full-length ³²P-labeled probe. The largest amount of message was present in heart, followed by the placenta and skeletal muscle, with smaller amounts present in brain, kidney, pancreas, lung and liver. Within the limits of resolution of the Northern blotting technique, only a single band was identified. Thus, the results indicate that the message identified by the inventors and shown in FIG. 12 is either full-length or nearly full-length. Multiple additional 5′-RACE reactions from multiple libraries did not identify any further sequence.

### EXAMPLE 3

This example illustrates analysis of the complete genomic organization of the iPLA2γ gene. To determine the genomic organization of the full-length iPLA₂γ gene, an examination was undertaken of sequence upstream from the putative start site of the BAC genomic clone (GenBank® accession number ACOO5 058).

Examination of the iPLA₂γ sequence upstream from the putative transcription start site did not reveal the presence of either TATA box, nor CAAT box consensus sequences. The full-length message of iPLA₂γ was aligned with BAC genomic clone RGO54DO4 sequence to determine the location of intron/exon boundaries. Two intron/exon boundaries were then identified within the 5′-untranslated region of the iPLA₂γ sequence utilizing conventional AG/GT splicing rules.

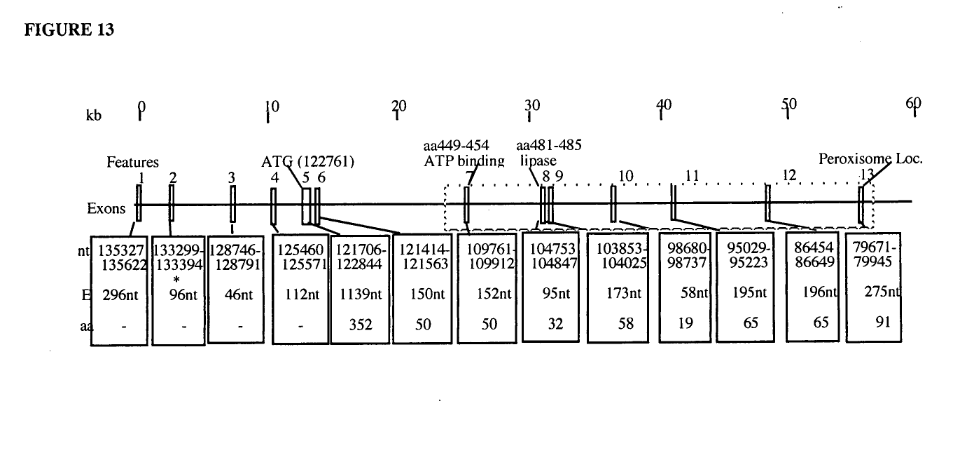

FIG. 13 is a schematic illustration of the human iPLA₂γ gene, deduced by determining the locations of intron/exon boundaries. Intron/exon boundaries of the iPLA₂γ gene are shown in scale (in kilobases). The thirteen (13) exons of the gene are indicated as open boxes. Spaces between the exons represent the relative sizes of the twelve (12) introns contained with in the gene. Regions of the gene that correspond to the ATP binding, lipase and peroxisomal localization consensus sequences are indicated in exons 7, 8 and 13, respectively. The dashed box enclosing exons 5-11 corresponds to previously identified exons in the BAC genomic clone (GenBank® accession number ACOO5058). The boxes at the bottom indicate the nucleotide numbers corresponding to the original BAC genomic clone report, with the sizes of each exon in nucleotides and in amino acids shown within.

More specifically, exon 1 of the iPLA₂γ gene is 346 nt in length, (nucleotides 135322-135627 of BAC genomic clone RGO54DO4) and is followed by a 1.9. kb intron. Exon 2 of the iPLA2γ gene is 96 nt in length, (nucleotides 133,299-133,394 of BAC genomic clone RGO54DO4) and is followed by a 4.5-kb intron. Exon 3 is 46 nt in length, (nucleotides 128,746-128,791 of BAC genomic clone RGO54DO4) and is followed by a 5.9-kb intron. Exon 4 of the iPLA2γ gene is 112 nt in length, (nucleotides 125571-125460 of BAC genomic clone RGO54DO4) and is followed by a 3.2 kb intron. The first candidate ATG start site occurs 84 nt downstream from the start of exon 5 (nucleotides 121,692-122,844 of BAC genomic clone RGO54DO4), which is 1151 nt in length and followed by a 139-nt intron 3. Exon 6 is 139 nt in length, (nucleotides 121,411-121,522 of BAC genomic clone RGO54DO4) and is followed by an 11.5-kb intron. Exon 7 begins the coding sequence previously reported in the GenBank® BAC clone report, as indicated in FIG. 13. Based on these findings, the iPLA2γ gene on chromosome 7 is ˜56 kb in size and contains 13 exons, ranging in size from 46 to 1139 nucleotides. Further, the results indicate that both the ATP binding motif (amino acid residues 449-454), and the lipase consensus sequence (amino acid residues 481-485) of the iPLA2γ gene require splicing of adjacent exons to form complete functional sequences. This splicing requirement distinguishes iPLA₂γ from the iPLA₂β, which contains its functional ATP and lipase sites within a single exon.

Still further, these results show that the promoter region of the iPLA_{2γ} gene contains neither a TATA box nor a CCAAT sequence within 400 nucleotides flanking the putative transcription start site. However, Tfsearch analysis reveals other promoter elements (e.g. several Spl/GC boxes) that are located in the promoter region, as well as several potential cap signals. One of the potential cap signals is located immediately upstream of the predicted transcription initiation site (i.e. at residues −6 to −13). TATA-less promoters are generally thought to typically respond to a variety of stimuli, with a range of transcription regulatory responses including differential expression during embryogenesis, tissue-specific distribution, and regulation by either viral or pharmacologic stimuli. Many TATA-less genes are growth-regulated, so that low levels of gene expression are seen in non-growing cells but then expression up-regulated as cells proliferate. Thus, these results provide a foundation for studies in which the identified promoter region is fused to a reporter gene and subsequently dissected to determine important transcriptional regulatory elements by deletional mutagenesis.

To investigate the possible existence of iPLA₂γ splice variants including alternative locations of intron/exon boundaries, further analysis of the iPLA₂γ sequence upstream from the putative transcription start site of the BAC genomic clone was undertaken. Three alternative splice variants were discovered. FIG. 2 is a schematic illustration of the three alternative splice variants of the iPLA₂γ gene, deduced in this further examination. A region of variation, including exons 2, 3 and 5 as identified above, is shown. Labeling indicates the size of corresponding protein in kDa, and length in nucleotides (nt). Open boxes are noncoding regions, shaded regions are putative coding regions, and stippled lines represent intron splicing. The three splice variants included a first, “Gamma 1”, that includes exon 2, exon 3 and a portion of exon 5 in noncoding region, with the start site in exon 5. A second, “Gamma 2” variant, includes exon 2 and a portion of exon 5 in noncoding region, but excludes exon 3 altogether. A third, “Gamma 3” variant, includes a new potential ATG start site in exon 2, and thus a portion of exon 2, all of exon 3 and a truncated exon 5 in coding region.

### EXAMPLE 4

To determine the molecular weight of the protein(s) translated by the iPLA₂γ message, the 2.4 kb PCR product as described above was expressed in an in vitro reticulocyte lysate translation system, and the molecular weights of the protein products determined.

For in vitro translation, a full-length iPLA₂γ construct in pcDNA1.1 (1 μg) was used in a coupled transcription/translation rabbit reticulocyte translation lysate system (Promega) with RNA synthesis from the T7 promoter of pcDNA1.1 using T7 RNA polymerase and translation in the presence of 20 (μ)Ci of ˜35 methionine for 90 minutes according to the manufacturer's instructions. More specifically, the full-length iPLA2γ PCR product was cloned into peDNA1.1 vector so that the T7 promoter region of pcDNA1.1 was upstream from the iPLA₂γ sequence. For coupled in vitro transcription/translation, RNA was synthesized from 1 μg of iPLA₂γ-pcDNAI1 in the presence of T7 RNA polymerase. Translation of RNA in the rabbit reticulocyte lysate system was performed in the presence of [35S] methionine, and following translation, Std of labeled product was boiled for two minutes in SDS loading buffer. Translated, radiolabeled protein products were then resolved by SDS gel electrophoresis and visualized by autoradiography.

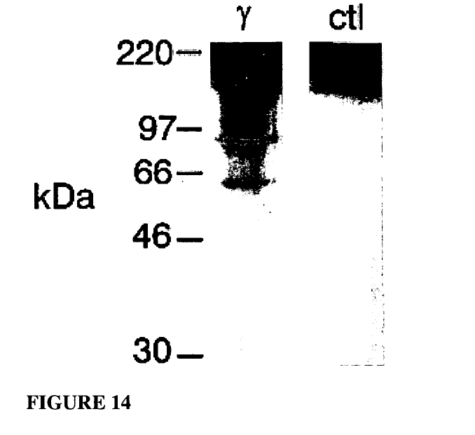

FIG. 14 shows the resolution by gel electrophoresis and visualization by autoradiography of two radiolabeled protein products of iPLA₂γ in vitro translation. In vitro translated product corresponding to iPLA₂γ is shown at left and indicated by “γ”, and a negative control indicated by “ctl” is shown at right. More specifically, FIG. 14 shows the resolution a first product corresponding to a molecular mass of approximately 77 kDa and a second corresponding to a molecular mass of approximately 63 kDa. Sizes of molecular weight standards in kDa are indicated at left.

The first radiolabeled protein product of about 77 kDa corresponds to translation initiation at methionine residue 101 of the full-length 88 kDa. The second radiolabeled protein product of about 63 kDa corresponds to translation initiation at methionine residue 221 of the full-length iPLA2γ.

### EXAMPLE 5

To compare the results of in vitro translation as described in Example 4, with translation in an intact eukaryotic cell, the translation of 2.4 kb iPLA₂γ in Sf9 cells was investigated.

This example thus illustrates the determination of the molecular weights of the two identified protein products of the iPLA₂γ gene as expressed in an intact eukaryotic cell, that the observed products correspond to results obtained in the in vitro translation as described in Example 4, and that iPLA₂γ protein products are present predominantly in the cell membrane.

Expression of iPLA₂γ in an intact eukaryotic system was evaluated using cultured Spodoptera frugiperda (Sf9) cells. Sf9 cells have been widely used to express recombinant proteins as a host cell in the baculovirus expression system. (See e.g. Liu et al., J. Immunol. 156:3292-300, 1996). It is believed that the translational elements of Sf9 cells exhibit a high degree of homology with mammalian translational elements. (See e.g. Sakahira et al., J. Biol. Chem. 274:15740-4, 1999; Kimball et al., J. Biol. Chem. 273:12841-5, 1998; Gu et al., Biochim. Biophys. Acta 1399:1-9, 1998). Sf9 cells were grown and infected with wild-type or recombinant baculovirus containing human phospholipase A₂γ cDNA as described in detail in Wolf, M. J. and Gross, R. W., J. Biol. Chem. 271:30879-85, 1996, which is herein incorporated by reference. Culture of Sf9 cells involved, in brief, placing Sf9 cells in growth medium in 100-ml spinner flasks equipped with a magnetic spinner. The rotation speed of the magnetic spinner was set at about 80 rpm, which is believed to be especially suitable for optimizing growth of Sf9 cells. The growth medium included Grace's medium supplemented with Yeastolate, Lactalbumin, 1% Antimycotic (all from Sigma), 10% fetal bovine serum and 1% Plutonic F-68 surfactant. Sf9 cells at a concentration of 1×106 cells/ml were prepared in 50 ml of the growth medium and incubated at 27° C. for one (1) hour prior to infection with either wild-type baculovirus or recombinant baculovirus containing human phospholipase A₂γ cDNA. After 48 hours, cells were repelleted by centrifugation, resuspended in ice-cold phosphate-buffered saline, and repelleted. All subsequent operation were performed at 4° C. The supernatant was decanted, and the cell pellet was resuspended in 5 ml homogenization buffer (25 mM imiazole, pH 8.0, 1 mM EGTA, 1 mM dithiothreitol, 0.34 M sucrose, 20 MM transepoxysuccinyl-L-leucylamido-(4-guanidino) butane, and 4 g/ml leupeptin). Cells were lysed at 0° C. by sonication (twenty (20) one-second bursts utilizing a Vibra-cell sonicator at a 30% output) and centrifuged at 100,000×g for one (1) hour. The supernatant containing predominantly cytosol was saved, and the membrane pellet was washed with homogenization buffer and resuspended using a Teflon homogenizer in 6 ml of homogenization buffer. After a brief sonication (ten (10) one-second bursts), the mixture was subjected to recentrifugation at 100,000×g for one (1) hour. After removal of the supernatant, the membrane pellet was resuspended in 1 ml homogenization buffer using a Teflon homogenizer and subsequently sonicated at 0° C. for five (5) one-second bursts.

The 2.4-kb message encoding iPLA₂γ as identified was inserted into the Sf9 cell vector pFASTBAC. Spinner cultures of Sf9 cells were infected with either the wild-type pFASTBAC (“Ctl) or pFASTBAC containing the 2.4-kb message (“γ”). At 48 hours post-infection, the cells were collected and cytosolic (“Cyto”) and membrane (“Memb”) fractions prepared as described above.

Sf9 cell cytosol and membrane fractions were loaded on a 10% polyacrylamide gel, resolved by SDS polyacrylamide gel electrophoresis, transferred to a polyvinylidene difluoride membrane, by electroelution in 10 mM CAPS, pH 11, containing 10% methanol. Dry powdered milk (5% w/v) in 20 mM Tris-HCl, pH 7.4, containing 137 mM NaCl and 0.1% Tween 20 was used to block non-specific binding sites before incubation with the primary antibody, immunoaffinity-purified antibody directed against iPLA₂γ, prepared as described below. Secondary antibody (anti-rabbit IgG horseradish peroxidase conjugate) was incubated with the blot for 1 hour and immunoreactive bands were visualized by ECL.

Immunoaffinity-purified polyclonal antibody directed against iPLA_{2γ} was prepared by immunizing rabbits with the iPLA₂γ synthetic peptide CENLIPLDESRNEKLDQ (SEQ ID NO:26). This peptide (G3, 4 mg), dissolved in minimal volume of dimethylsulfoxide, was conjugated to maleimide-activated keyhole limpet hemocyanin (KLH, 2 mg) in 200 td of 83 mM sodium phosphate, 0.9 M NaCl, and 0.1 mM EDTA at 22° C. for two hours. After extensive dialysis against 83 mM sodium phosphate containing 0.9 mM NaCl, a 1:1 emulsion was made with the peptide-KLH conjugate and Freund's complete adjuvant. This emulsion was then injected subcutaneously into two anaesthetized New Zealand white rabbits. Two booster injections of a 1:1 emulsion of the peptide-KLH conjugate and Freund's incomplete adjuvant were given two and four weeks after the initial immunization, and then serum was collected. A peptide affinity column was generated by coupling the iPLA₂γ synthetic peptide to a thiopropyl-Sepharose (Amersham-Pharmacia) resin in the presence of 100 mM sodium citrate, pH 4.5. The resin-peptide suspension was mixed by inversion overnight at 22° C. Coupling efficiency was monitored by the displacement of the 2-pyridyl sulfide groups as 2-thippyridone through spectrophotometric monitoring at 343 nm. Before chromatography, the resin-peptide suspension was equilibrated with 10 mMTris-HCl, pH 7.5 in an Econo-pac column (BioRad) by gravity flow. Serum containing the anti iPLA₂γ antibodies was diluted 1:10 (v/v) with 10 mM Tris-HCl, pH 7.5 and then applied to the peptide affinity column by gravity flow. The diluted serum was then re-applied twice to the column to assure binding of the antibody to the column. After washing the column extensively with 10 mM tris-HCl, pH 7.5 containing 500 mM NaCl, bound anti-iPLA₂γ antibody was eluted from the column with 100 mM glycine, pH 2.5 and the immediately neutralized with 100 mM Tris-HCl, pH 9.0. The antibody was then concentrated using Centricon-10 (Americon) centrifugal filter devices and stored at 40° C. until use.

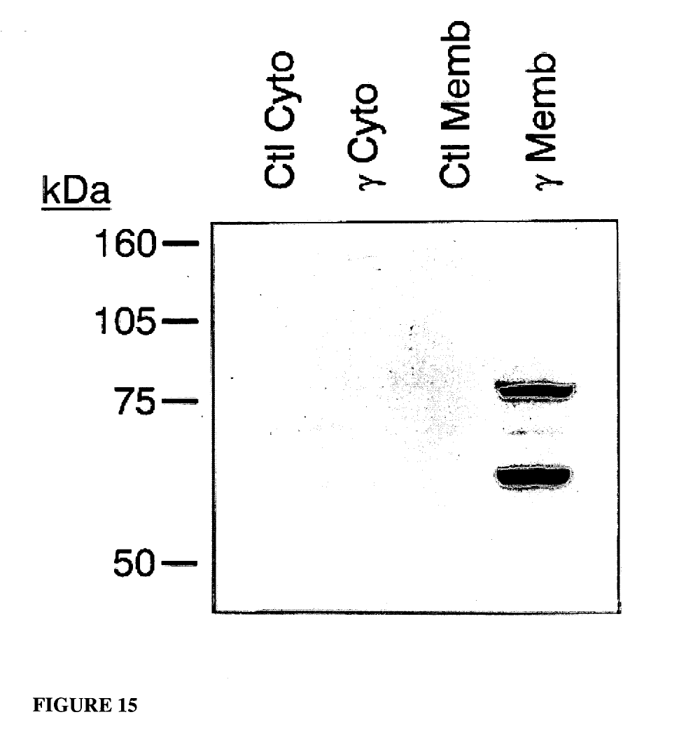

FIG. 15 shows the results of Western analysis of iPLA₂γ expression in Sf9 cells, and confirm in an intact eukaryotic system the identification of translation initiation sites at methionine residues 101 and 221 as identified in the in vitro system as described in Example 4. The results as shown in FIG. 15 are typical of three independent experiments. Specifically, Western analysis of membrane fractions from Sf9 cells infected with vector harboring iPLA₂γ (“γ Memb”) demonstrated the presence of two major bands corresponding to molecular masses of 77 and 63 kDa. However, no such products were present in membrane fractions from Sf9 cultures infected with wild-type virus (“Ctl Memb”). In contrast, cytosolic fractions from control or iPLA2γ-transfected Sf9 cells did not contain any detectable immunoreactive protein. Collectively, these results support the existence of two translation initiation start sites at methionine residues 101 and 221.

### EXAMPLE 6

To determine whether iPLA₂γ translation products as described in Example 5 are biologically active, a phospholipase enzyme activity analysis was undertaken of Sf9 cells infected with recombinant baculovirus encoding iPLA₂γ. This example demonstrates that iPLA₂γ translation products are biologically active and more specifically, are catalysts of cleavage of the sn-2-fatty acid of choline glycerophospholipids.

Briefly, Sf9 cells were infected with either wild-type baculovirus or baculovirus encoding human iPLA_{2γ}, as described in Example 5. Cytosolic and membrane fractions of the infected Sf9 cells were prepared as described above. Phospholipase A₂ activity was assessed as follows. Membrane fractions from control cells or cells expressing iPLA₂γ were incubated with 40 μM L-α-1-palmitoyl-2-[1-¹⁴C]oleoyl phosphatidylcholine in 100 mM Tris-Hac, pH 8.0, containing 1 mM EGTA at 37° C. Aliquots of the reaction were removed at the indicated times, and the amount of [1-¹⁴C]oleic acid released was quantified as described above. Specifically, the release of sn-2-fatty acid from glycero-3-phosphocholine was determined as a function of time.

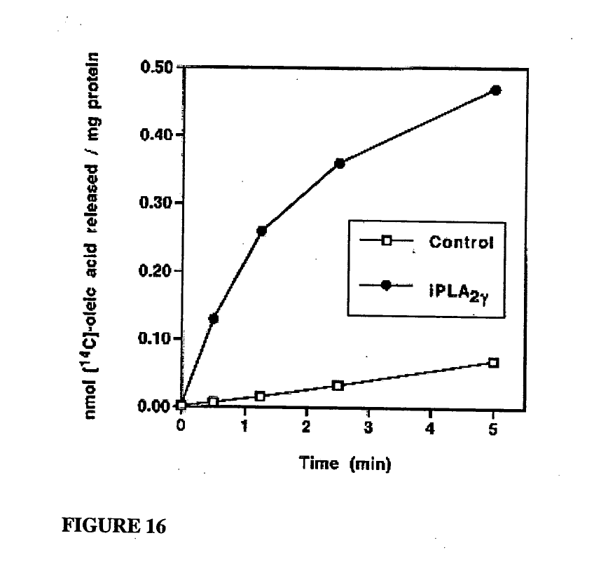

FIG. 16 is a graph showing an initial rate analysis of phospholipase activity in Sf9 cells infected with recombinant baculovirus encoding iPLA₂γ. The results shown in FIG. 16 are representative of three independent experiments.

Membrane fractions from cells infected with vector harboring the iPLA_{2γ} insert released fatty acid from the sn-2-position of phosphatidylcholine approximately 10-fold faster than from membrane fractions prepared control cells infected with wild-type vector. Liberation of radiolabeled fatty acid was nearly linear for 2 minutes at a velocity of approximately 0.3 nmol/mg-min. Phospholipase A₂ activity was entirely calcium independent in the range of 0-10 mM and demonstrated a pH optimum at pH 8.0 (not shown), which is at or near physiologic pH. In contrast, phospholipase A₂ activities in cytosolic fractions of cells infected with wild-type vector and from cells infected with vector harboring the iPLA₂γ insert were found to be similar (not shown).

To determine the substrate selectivity of the phospholipase A₂ activity demonstrated in Sf9 cells infected recombinant baculovirus encoding iPLA₂γ multiple alternative phospholipase A2 substrates were tested. Membrane fractions from both groups of cells were incubated in 100 mM Tris-HCl, pH 8.0, containing 1 mM EGTA for 2 minutes at 37° C. in the presence of either 40 μML-α-1-dipalmitoyl-2-[1-¹⁴C] phosphatidylcholine (“PPPC”), L-α-1-palmitoyl-2-[1-¹⁴C]oleoyl phosphatidylcholine (“POPC”), L-α-1-palmitoyl-2-[1-¹⁴C] linoleoyl phosphatidylcholine (“PLPC”), or L-α-1-palmitoyl-2-[1-¹⁴C] arachidonyl phosphatidylcholine (“PAPC”). Reactions were terminated by butanol extraction, and radiolabeled fatty acids were resolved by TLC and quantitated by scintillation spectrometry.

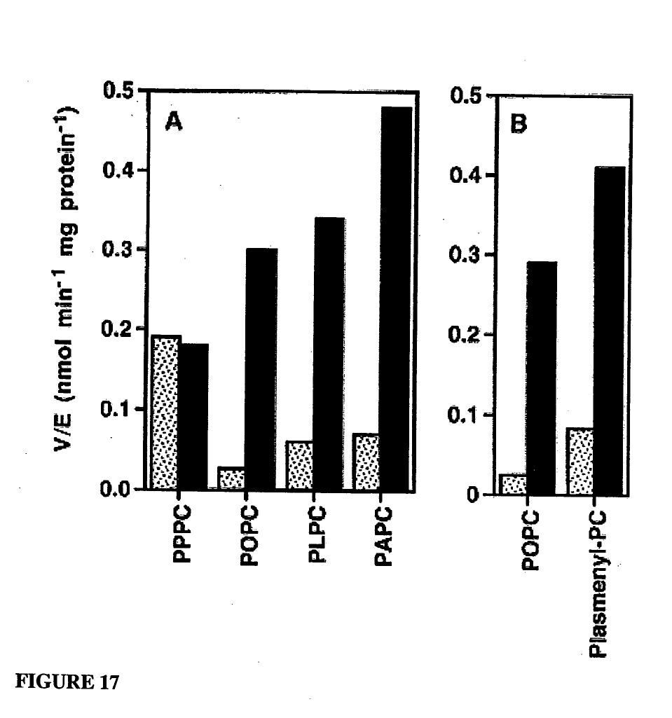

FIG. 17 is a substrate selectivity profile of iPLA₂γ phospholipase A₂ activity in Sf9 cells infected recombinant baculovirus encoding iPLA_{2γ}. Panel A of this figure shows the results of phospholipase A₂ activity analysis from Sf9 cells infected with either wild-type baculovirus (stippled bars) or recombinant baculovirus encoding iPLA₂γ (solid bars). The results are representative of three independent experiments. Thus, incubation of membrane fractions containing iPLA₂γ with phospholipids containing three distinct types of unsaturated fatty acids at the sn-2-position (oleic, linoleic, and arachidonic acids) gave similar specific activities that were each 6-10 times greater than activities manifest in membrane preparations derived from cells infected with wild-type vector.

FIG. 17, Panel B shows the results of further phospholipase A₂ activity analyses undertaken to identify the initial site of hydrolysis of phospholipids by iPLA_{2γ}. Membranes derived from Sf9 infected with either wild-type baculovirus (stippled bars) or recombinant baculovirus encoding iPLA₂γ (solid bars) were incubated with either 40 μM L-α-1-palmitoyl-2-[1-¹⁴C]oleoyl phosphatidylcholine (“POPC”), or 40 μM synthetic radiolabeled 1-O-(Z)-hexadec-1′-enyl-2-[9,10-3H]octadec-9′-enoyl-sn-glycero-3-phosphocholine (“plasmenyl-PC”), under incubation conditions as described for Panel A. Plasmenyl choline has a relatively enzymatically resistant vinyl ether linkage at sn-1.

Panel B of FIG. 17 shows that the rate of hydrolysis using plasmenyl choline was similar to that using sn-2-[1-¹⁴C]phosphatidylcholines, thus supporting the conclusion that hydrolysis of substrate by iPLA2γ occurs predominantly at the sn-2-position. Therefore, iPLA₂γ gene products are catalysts of cleavage of the fatty acid of choline glycerophospholipids.

### EXAMPLE 7

To establish iPLA₂γ as a member of the calcium-independent phospholipase _{A2} family, the inhibition of the phospholipase A₂ activity of iPLA₂γ by (E)-6-(bromomethylene)-3-(1-napthalenyl)-2H-tetrahydropyran-2-one (BEL) was studied.

(E)-6-(bromomethylene)-3-(1-napthalenyl)-2H-tetrahydropyran-2-one (BEL) has previously been shown to be a powerful and irreversible mechanism-based inhibitor of myocardial cytosolic and membrane-associated iPLA2, with nearly complete inhibition of myocardial cytosolic membrane-associated iPLA2 at concentrations of approximately 2-5 μM. (Hazen et al., J. Biol. Chem. 266:7227-32, 1991; Zupan et al., J. Med. Chem. 36: 95-100, 1991; Gross et al., Biochemistry 32:327-36, 1993; Ramanadham et al., Biochemistry 32:337-46, 1993, which are herein incorporated by reference).

Briefly, Sf9 cells were infected with either wild-type baculovirus or baculovirus encoding human iPLA₂γ, as described in Example 5. Membrane fractions of the infected Sf9 cells were prepared as described above, and then incubated for three minutes at room temperature in 100 mM Tris-HAc, pH 8.0, containing lmM EGTA, with varying concentrations of BEL, or ethanol vehicle, as indicated along the x-axis. L-α-1-palmitoyl-2-[1-¹⁴C]oleoyl phosphatidylcholine (40 μM final concentration) in ethanol was then added to each reaction, followed by incubation at 37° C. for two minutes. Released [1-¹⁴C]oleic acid was quantitated as described above.

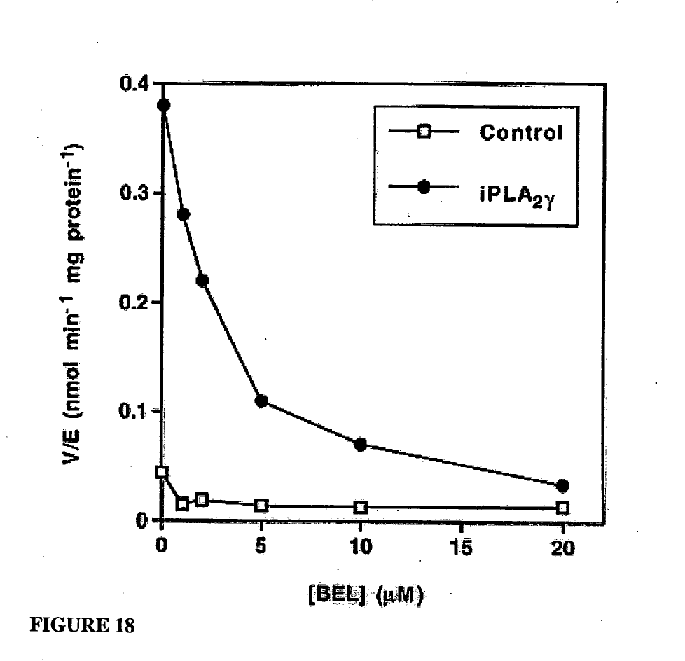

FIG. 18 is a graph showing data of inhibition of the phospholipase A₂ activity of iPLA₂γ by (E)-6-(bromomethylene)-3-(1-napthalenyl)-2H-tetrahydropyran-2-one (BEL). The results shown in FIG. 18 were representative of three independent tests.

As shown in FIG. 18, preincubation of membranes harboring iPLA_{2γ} with 5 μM BEL for three minutes prior to the addition of substrate resulted in the inhibition of approximately 70% of iPLA₂γ phospholipase A₂ activity. The IC50 for BEL inhibition of iPLA₂γ was approximately 3 μM. Thus, the results establish that BEL is an effective inhibitor of iPLA₂γ.

Previous work has characterized iPLA₂ activity in certain tissues, as well as a calcium-facilitated phospholipase A₂γ. Specifically, prior studies have identified iPLA₂ activity in cytosolic and membrane fractions of canine and human myocardium. (Wolf, R. A. and Gross, R. W., J. Biol. Chem. 260:7295-303, 1984; Hazen, S. L. and Gross, R. W., Circ. Res. 70:486-95, 1992, which are herein incorporated by reference). Additional studies have demonstrated an increase in membrane-associated, BEL-inhibitable iPLA₂ activity during myocardial ischemia or hypoxia. (Hazen et al., J. Biol. Chem. 266:5629-33, 1991; Ford et al., J. Clin. Inv. 88:331-35, 1991; McHowat J. and Creer, M. H., Lipids 33:1203-12, 1998; McHowat et al., Am. J. Physiol. 274:C1727-37, 1998, which are herein incorporated by reference). It has also been proposed that increased myocardial iPLA2 activity during ischemia contributes to ventricular arrhythmias and hemodynamic dysfunction through the production of lysophospholipids and arachidonic acid, each of which has potent electrophysiological effects. (Wolf, R. A and Gross, R. W., supra; Gross, Trends Cardiovasc. Med. 26:115-21, 1992; and Gross, Biochem. Soc. Trans. 26:345-49, 1984, which are herein incorporated by reference).

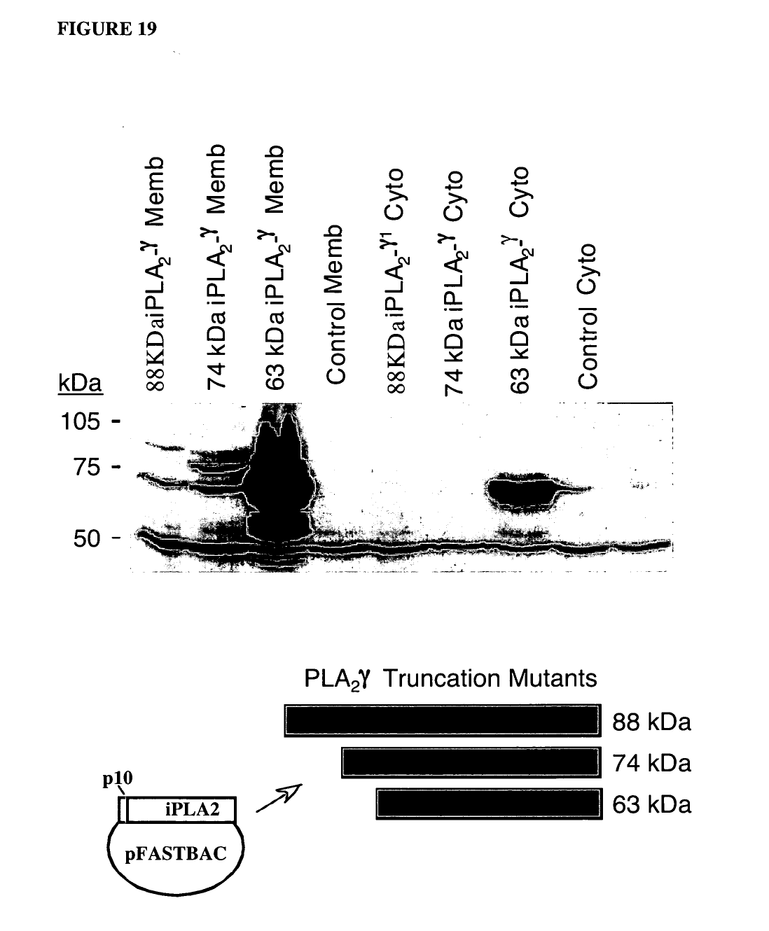

A calcium-facilitated phospholipase A₂γ shares homology with the catalytic domain of calcium-facilitated phospholipase A₂α and has been cloned and expressed. Northern analysis has demonstrated the presence of calcium-facilitated phospholipase A₂γ message in skeletal and heart muscle, and cloning and expression of calcium-facilitated phospholipase A₂γ in Sf9 cells has confirmed the membrane localization and calcium-independence of calcium-facilitated phospholipase A₂γ. (Underwood et al., J. Bio. Chem. 273:21926-32,1998 which is herein incorporated by reference). However, calcium-facilitated phospholipase A₂γ is not inhibited by BEL at <50 μM [BEL] (C. M. Jenkins, D. J. Mancuso, X. Han and R. W. Gross, “Identification of Calcium-independent Phospholipase A₂ (iPLA₂) β, and Not iPLA_{2γ}, as the Mediator of Arginine Vasopressin-induced Arachidonic Acid Release in A-10 Smooth Muscle Cells”, The Journal of Biological Chemistry, Vol. 277, No. 36, Issue of September 6, pp. 32807-32814, 2002 which is herein incorporated by reference in its entirety) and is therefore clearly distinguishable from iPLA_{2γ}, which is subject to potent inhibition by BEL as shown in FIG. 19. In contrast, the large majority of iPLA₂γ activity from ischemic hearts or hypoxic myocytes in culture is membrane-associated and exquisitely sensitive to BEL inhibition. Accordingly, the inventors herein have succeeded in identifying iPLA₂γ as a likely polypeptide for catalyzing the increase in ischemia-induced iPLA₂γ activity. Further, the results suggest that the peroxisomal compartment is a source of regulatory and potentially arrythmogenic eicosanoid metabolites and lysophospholipids.

### EXAMPLE 8

To determine whether iPLA₂γ expression is subject to translational control, multiple iPLA₂γ truncation mutants were prepared and their expression in Sf9 cells studied.

To prepare truncated iPLA₂γ constructs in pFASTBAC, PCR primers containing appropriate restriction sites for cloning were used, and performed as described above. For example, the original full-length 88 kDa construct was used as a template in PCR with a sense primer annealing at nt 650-676 paired with reverse primer M458 (5′-GCATAGCATGCTCACAATTTTGAAAAGAATGCAAGTCC-3′) (SEQ ID NO:12) to create a truncated iPLA₂γ (63 kDapF) with a methionine start site corresponding to amino acid 221 in the full-length protein. Expression of this construct in the baculovirus system as described above resulted in a 63 kDa recombinant protein. A second construct was created with a start site corresponding to methionine 122 to produce a 74 kDa recombinant protein. Additional constructs were also prepared as described above to produce N-terminal truncated recombinant proteins of approximately 77, 70 and 66 kDa that were used in additional studies of translational inhibition as described below in Example 9. Further, the same methods were used to for segments of iPLA_{2γ}5′ sequence ranging between 15-663 nt which were inserted 5′ of the insert sequence in 63 kDapF to identify potential sequences involved in translational regulation, as described below in Example 10.

The expression of the 63 kDa and the 74 kDa truncation mutants were studied in Sf9 cells using the expression system as described above. Cytosolic and membrane fractions were obtained as described above, and a Western analysis performed on the polypeptide products of the 63 kDa and 74 kDa truncation mutants of iPLA₂γ, as expressed in cytosolic and membrane fractions prepared from Sf9 cells.

FIG. 19 shows the results of the Western analysis. Both cytosolic and membrane fractions (“63 kDa iPLA_{2γ}Cyto” and “63 kDa iPLA₂γ Memb”, respectively) from Sf9 cells infected with vector harboring truncation mutants coding the 63 kDa product of iPLA₂γ, demonstrated the presence of major bands corresponding to molecular mass of 63 kDa, though to a lesser extent in the cytosolic fraction. However, analysis of both cytosolic and membrane fractions (“74 kDa iPLA_{2γ}Cyto” and “74 kDa iPLA_{2γ}Memb”, respectively) from Sf9 cells infected with vector harboring truncation mutants coding the 74 kDa product of iPLA₂γ, demonstrated greatly decreased amounts of product in comparison to that expressed using the 63 kDa truncation mutant. Similarly, analysis of both cytosolic and membrane fractions (“iPLA₂γCyto” and “iPLA_{2γ}Memb”, respectively) from Sf9 cells infected with vector harboring truncation mutants coding the iPLA₂γ (i.e. wild-type 88 kDa protein), demonstrated little or no expressed product in comparison to that expressed using the 63 kDa truncation mutant. Collectively these results support the existence of a sequence or sequences involved in translation inhibition. Further, such sequences can be localized to the stretch of sequence representing the difference between the 63 kDa truncation mutant and the 74 kDa truncation mutant.

### EXAMPLE 9

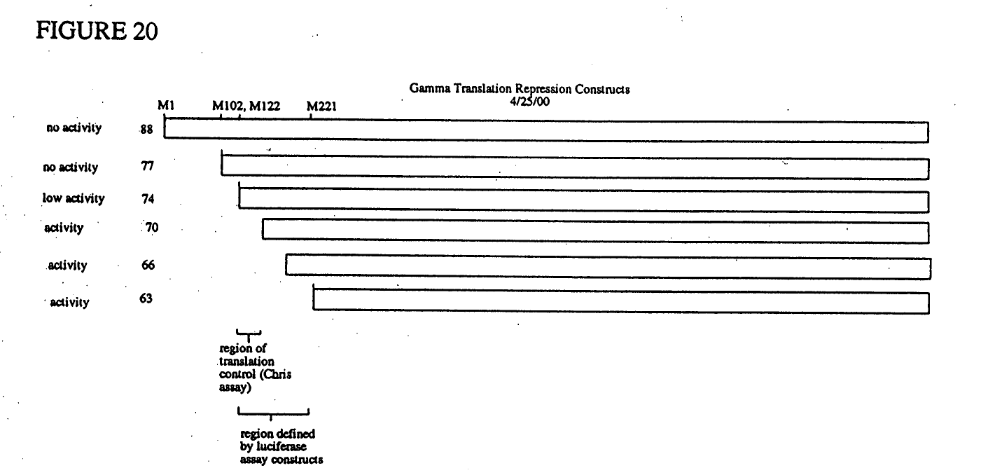

To more precisely localize the sequence involved in transcriptional and/or translational inhibition of iPLA₂γ, additional constructs (70 kDa and 66 kDa) were prepared utilizing the methods described above that were used for preparation of the 88 kDa, 77 kDa, 74 kDa, and 63 kDa in vector pFASTBAC for baculoviral expression. Unlike the 88 kDa, 77 kDa, 74 kDa, and 63 kDa constructs which correspond to authentic ATG start sites in iPLA_{2γ}, artificial ATG start sites were introduced to synthesize the 70 kDa and 66 kDa constructs. In brief, sense PCR primers were prepared which were downstream from the 74 kDa start site and contained a restriction enzyme site (Sall) followed by an ATG start site. The primer for the 70 kDa construct was M33636 (5′-AAAAGTCGACATGAAGCCATCAAATCTC-3′) (SEQ ID NO: 55) corresponding to an ATG start site at nucleotide 452 of SEQ ID NO: 13. The primer for the 66 kDa construct was M33637 (5′-AAGAGTCGACATGAGGTAAACGCAGTC-3′) (SEQ ID NO: 56) corresponding to an ATG start site at nucleotide 549 of SEQ ID NO: 13. These primers was paired with M458 (SEQ ID NO: 12) to amplify products of 1.9 kb and 1.8 kb, respectively for the 70 kDa and 66 kDa constructs, from the full-length 88 kDapF construct used as a template. PCR products were then cloned via SalI/SphI restriction sites into the polylinker region of pFASTBAC vector for use in the baculoviral expression system as described earlier. FIG. 20 is a schematic illustration of each of the polypeptides products. The rectangular block at the top represents the full-length 88 kDa iPLA2γwith the locations of alternative initiator methionine residues M1, M102, M122, and M221 indicated. Below this rectangle are represented each of the truncated constructs prepared, with the left side of the block representing the degree of truncation in reference to the full-length 88 kDa iPLA₂γ. The relative amount of activity of each of these constructs as assayed in the phospholipase A₂ assay described earlier is indicated on the left of each representation of the constructs. Essentially no or very little phospholipase A₂ activity was measured under the assay conditions used until iPLA₂γ sequence was truncated to the 70 kDa construct and high activity was measured for the 66 kDa as well as for the 63 kDa constructs. These results suggested that an inhibitory sequence lay in the region between the 5′ start of the 74 kDa and 70 kDa constructs. This region is represented by the smaller brackets at the bottom of the figure. Further, no immunoreactive band was detectable from cells containing the 77 kDa truncation mutant or the 88 kDa full-length sequence. The larger brackets indicate the results of other studies in which the location of the inhibitory region was defined by using iPLA₂γ sequence placed upstream of the luciferase gene in a vector construct to direct luciferase expression in transfected CV1 cells.

### EXAMPLE 10

To further identify and locate the sequences involved in translational inhibition of iPLA₂γ, the effect on translation of several iPLA_{2γ} 23-mer sequences 5′ of the 63 kDa sequence was studied.

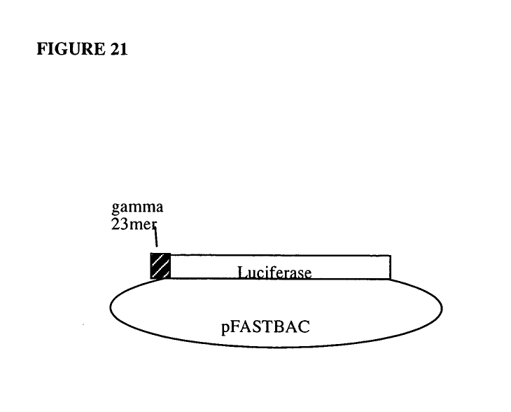

Briefly, sequences from iPLA_{2γ} were inserted by ligation of 15-23-mer annealed phosphorylated oligonucleotide pairs 5′ of full-length luciferase coding sequence which was cloned into pFASTBAC via NotI/XbaI restriction sites. Luciferase activity of recombinant protein produced in the Sf9 system was then measured using the Luciferase Assay System of Promega. (Wood, K. V., (1991) In: Bioluminescence and Chemiluminescence: Current Status, Stanley, P. and Kricka, L. eds., John Wiley and Sons, Chichester, N.Y., 543; and Luciferase Assay System Technical Bulletintus, #TB 101, Promega Corporation, 1996, which are herein incorporated by reference). FIG. 21 is a schematic diagram showing ligation of 23-mer iPLA₂γ sequence to luciferase coding sequence and cloned into pFASTBAC.

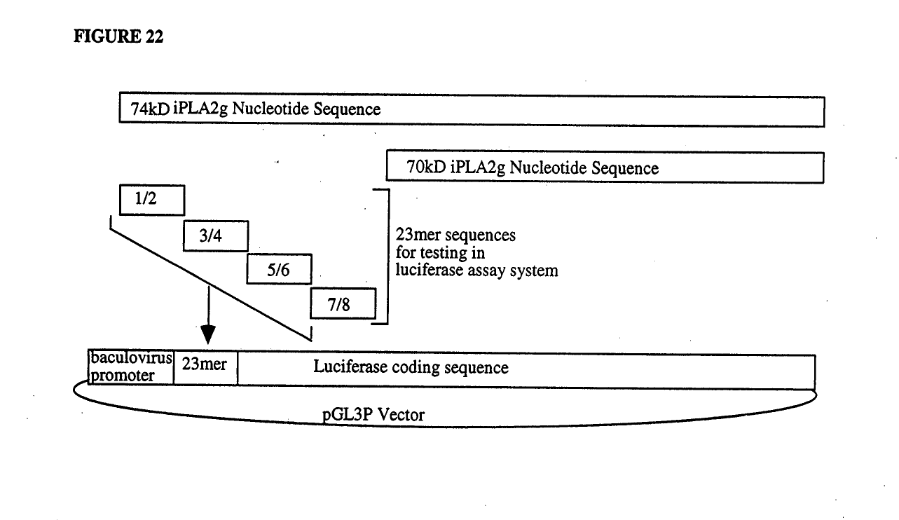

FIG. 22 is a schematic representation of the location of the 23-mer sequences studied. Schematic representations of the 74 kDa protein product of iPLA₂γ as well as the 70 kDa sequence, are shown at the top for comparison. “½”, “¾”. “⅚”, and “⅞” each indicate successive 23-mer DNA sequences taken from coding region representing the difference between the sequences for a 70 kDa iPLA₂γ protein and the 74 kDa protein. The numbers 1-8 indicate the primers that were used to amplify each 23-mer piece. Constructs were prepared by fusing luciferase coding sequence to a one of the four alternative 23mer sequences from iPLA_{2γ}, and to the baculovirus promoter. Four alternative constructs, each containing one of the four different 23mer sequences from the 74 kDa iPLA₂γ truncation mutant, were prepared and then expressed in Sf9 cells as described above. After 48 hours allowed for expression, cell extracts were prepared and then tested for level of expression using the luciferase assay system as described in detail above.

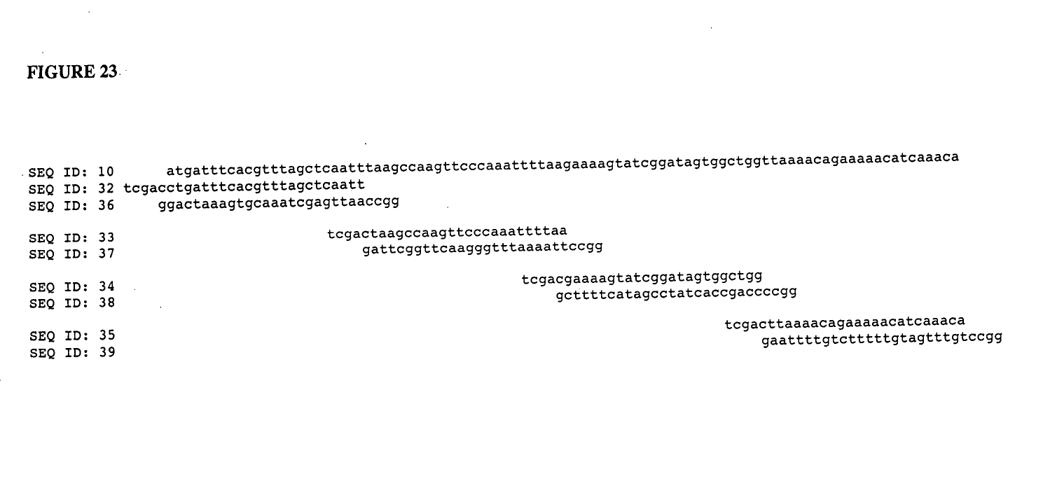

FIG. 23 shows the phosphorylated oligo pairs for sequence between nucleotide 364-455 that is involved in translation inhibition of iPLA₂γ in the luciferase expression system, thus identifying four pairs, ½”, “¾”. “⅚”, and “⅞” which each indicate successive 23-mer DNA sequences taken from coding region representing between the N terminus of the 70 kDa iPLA₂γ protein and the 74 kDa protein.

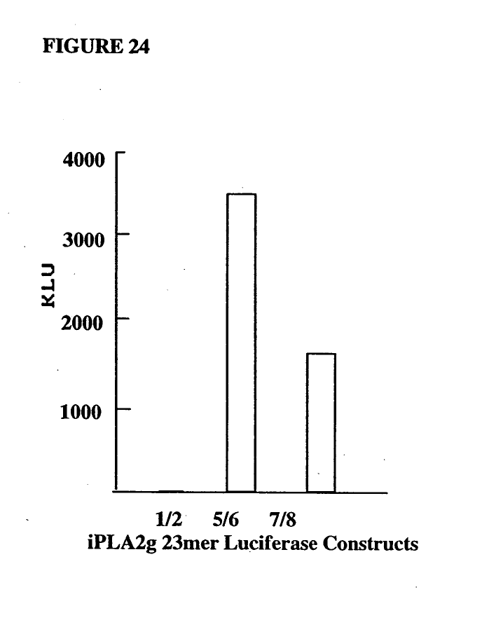

FIG. 24 is a bar graph showing luciferase assay results of the expression of three of the four different iPLA₂γ constructs. The graph plots relative light units (RLU) emitted for each of three constructs containing a different 23mer sequence from iPLA_{2γ} 74 kDa truncation mutant, as described above. The first construct contained the 23-mer sequence identified as “½” on the x-axis, the second construct, contained the 23-mer sequence identified as “⅚” on the x-axis, and the third construct contained the 23-mer identified as “⅞” on the x-axis. The ¾ sequence did not amplify. The results, including little or no expression of the construct containing the ½ sequence, thus show that at least a portion of ½ is involved in translational inhibition of iPLA₂γ. In contrast, the construct containing ⅚ resulted in a relatively high level of expression of iPLA₂γ, showing ⅚ is unlikely to be involved in translational inhibition. The results for the third construct containing ⅞ showed an intermediate level of expression that was not significantly different from the ⅚ construct. Thus, ⅞ is not likely to be involved in translational inhibition of iPLA₂γ. Thus, these results show that iPLA₂γ is subject to translational inhibition by the ½ 23-mer piece taken from coding region representing the difference between the sequences for a 70 kDa iPLA₂γ protein and the 74 kDa protein.

### EXAMPLE 11

To better understand the role of iPLA2γ in an intact animal, mice over-expressing iPLA₂γ were prepared by exploiting the cardiomyocyte specificity of the α-mhc promoter. Briefly, we have engineered SalI sites on the 5′ and 3′ ends of iPLA₂γ by PCR. The SalI digested fragment was cloned into Sall digested and alkaline phosphatase treated a ci-mhc vector and sequenced in both directions. A Not I fragment containing the α-mhc promoter in tandem with the iPLA₂γ sequence was submitted to the Washington University Neuroscience Transgenic facility for microinjection of DNA directly into the pronuclei of mouse (B6CBAF1/J) zygotes which will result in integration of the transgene into the mouse germ line.

After weaning, founder mice will be identified by PCR analysis of mouse tail DNA utilizing specific primers which amplify 600 nt product containing the iPLA₂γ sequence. The founder mice will be bred with WT B6CBAF1/J mice. Offspring harboring the iPLA₂γ sequence overexpressed in a cardiac myocyte specific fashion will be identified by tail DNA PCR analysis. Offspring harboring the gene will be bred and the degree of iPLA₂γ overexpression will be determined by quantitative PCR, Western blotting, and fluorescent measurement of tissue sections.

Lipids were extracted from mouse myocardium (˜50 mg) by a modified Bligh and Dyer technique utilizing 50 mM LiCl in the aqueous layer in the presence of internal standards selected based on their lack of demonstrable endogenous molecular ions in that region(1-3). ESI/MS analysis was performed utilizing a Finnigan TSQ-7000 Spectrometer as previously described (1-3).

Surprisingly, phosphatidyicholine, ethanolamine glycerophospholipids, and plasmaogen levels in fed transgenic mice were each reduced by about 60% of similarly fed WT mice. (FIG. 26) Triacylglyceride levels of fed transgenic vs WT mice were moderately increased (1.7 fold). Upon fasting in WT mice, levels of phosphatidylcholine, ethanolamine glycerophospholipids, and plasmaogen were reduced by 17% with no significant change in triacylglyceride levels. In contrast, no significant alterations of phosphatidylcholine, ethanolamine glycerophospholipids, and plasmaogen levels were detected upon fasting of transgenic mice. However, triacylglyceride levels increased 10 fold resulting in a 2 fold increase in total lipid mass. This increase was 1.9× and 1.6× that of WT fasted and fed mice, respectively (FIG. 26).

Accumulation of triacylglyceride in myocardial tissue has been shown to be associated with increased risk of cardiac dysfunction (Unger and Orei FASEB J 15: 312-321, 2001) resulting in ‘lipotoxic heart’ disease. Diabetes is also associated with increased triacylglyceride accumulation in the heart as well as alterations in lipid metabolism. The accumulation of triacylglycerides and associated membrane lipid alterations in iPLA_{2γ} transgenic mice (particularly under fasting conditions) is anticipated to contribute to an increased susceptibility for development of cardiomyopathy in this animal model. Studies of iPLA₂γ transgenic mice have relevance with regard to the functional role of iPLA₂γ in living organisms. These studies imply that iPLA₂γ plays an important role in maintenance of normal lipid homeostasis in the heart and that dysfunction of iPLA₂γ may lead to a predisposition for disorders of lipid metabolism, development of cardiomyopathy, and susceptibility to diabetic cardiomyopathy. The iPLA₂γ transgenic mouse should be a useful model for study of inherited and acquired lipid disorders and further analysis of the role of triacylglyceride accumulation in diabetic and other metabolic associated cardiomyopathy. The iPLA_{2γ} transgenic mice will also be a useful model to explore therapies for disorders associated with accumulation of triacylglycerides in the heart. In particular, therapies directed toward modulation of myocardial iPLA₂γ levels will be relevant.

References:

1. Han, X. & Gross, R. W. Electrospray ionization mass spectroscopic analysis of human erythrocyte plasma membrane phospholipids. Proc. Natl. Acad Sci. USA 91, 10635-10639 (1994).

2. Han, X. and Gross, R. W. Structural determination of lysophospholipid regioisomers by electrospray ionization tandem mass spectroscopy. J. Am. Chem. Soc. 118, 451-457 (1996).

3. Han, X., Gubitosi-Klug, R. A., Collins, B. J., and Gross, R. W. Alterations in individual molecular species of human platelet phospholipids during thrombin stimulation: Electrospray ionization mass spectrometry-facilitated identification of the boundary conditions for the magnitude and selectivity of thrombin-induced platelet phospholipid hydrolysis. Biochemistry 35, 5822-5832 (1996).

### EXAMPLE 12

To examine the stability of iPLA₂γ message expressed in the baculoviral system, spinners (100 ml) were infected with equivalent volumes of each truncated viral iPLA₂γ construct (m.o.i.=1) and 48 h later, actinomycin D was added to a concentration of 10 g/ml. Following actinomycin D addition, aliquots (2 ml) were removed, centrifuged to collect pellets, and placed on ice at 0, 15, 30, 60, 120, and 240 mm. RNA was prepared from each pellet following quick freezing in liquid N2 following the RNeasy (Qiagen) protocol. Recovery of RNA was determined spectrophotometrically at 260 nm. RNA samples (2 μg) were fractionated on a 10% polyacrylamide Lattitude RNA midi gel (BioWhittaker) and blotted onto a nylon membrane, cross-linked by exposure to a UV light source for 1.5 mm, and then baked at 85° C. for 60 mm. After prehybridization in ExpressHyb hybridization buffer (Clontech) for 30 min, the blot was hybridized 1h at 68° C. with a full-length iPLA₂γ random-primed [³²P]dCTP in a minimal volume of hybridization solution, and washed with 2×SSC containing 0.1% SDS twice for 30 min each, followed by two washes with 0.1×SSC containing 0.1% SDS for 40 min each at 50° C., as described in the manufacturer's instructions. Hybridized sequences were identified by autoradiography for 16 h.

The 63 kD construct expressed over 10 fold more iPLA₂γ message than either the 88 kD or 74 kD constructs. Message levels appeared to be similarly stable over a 4 h period (FIG. 27).

### EXAMPLE 13

To study the regulated expression of in vitro translated truncated iPLA₂γ constructs, PCR primers were designed for PCR amplification of sequences for the full length (88 kDa) and for each of the truncated constructs (77, 74 and 63 kDa) and cloning via EcoRV and NotI sites into the polylinker site of vector pEF vector (Invitrogen). The methodology used is the same as that described earlier in sections 129-132 and 198.

The truncated iPLA₂γ constructs in vector pEF (Invitrogen) were used in a TnT Quick Coupled Transcription/Translation System (Promega) as described by the manufacturer Proteins were separated by SDS PAGE, the gels dried, and exposed to autoradiography.

Truncation resulted in enhanced expression of iPLA₂γ in the mammalian in vitro expression system. Expression of truncated constructs 77 kD, 74 kD, 7OkD, and 63 kD were over 10 fold greater than expression of the full-length 88 kD construct (FIG. 29). These results, in concert with results from expression in the baculoviral expression system suggest the presence of an inhibitory sequence between the 88 kD and 63 kD ATG start sites.

### EXAMPLE 14

Identification of Transcriptional Regulatory Elements in the 5′ Coding Region of iPLA₂γ. The observed differences in baculoviral expression patterns of the sequentially truncated iPLA₂γ message and resultant polypeptide synthesis from the four AUG start sites suggested the presence of a transcriptional inhibitory sequence between nucleic acid residues corresponding to the 88 kDa and 63 kDa AUG start sites. To localize the regulatory domain upstream of the 63 kDa start site of iPLA₂γ responsible for transcriptional repression, PCR primers in Table III were used to amplify segments containing 124 nt of sequence upstream of the iPLA_{2γ} 63 kDa start site. All 3′ PCR primers in Table III were designed to generate identical upstream Kozak (GCCACCATG) sequences upstream from the ATG start. In each case the sequence around the ATG start is“GCCAXCATG” (where ‘X’ is a ‘C’ nucleotide in all constructs except 83 which contains an ‘A’ nucleotide). In each case, PCR products were cloned into HindIII/NcoI restriction sites within the polylinker region of pGL3 promoter vector (pGL3P). Additionally, because of the presence of a naturally occurring NcoI site within the 83 construct, an AflIII restriction site was utilized at the 3′ end of this construct (instead of NcoI) to generate a compatible cohesive end for cloning into the NcoI restriction site of pGL3P. Transient transfection of CV-1 cells with each of the inhibitory constructs was performed using Lipofectamine Plus (Invitrogen). For each transfection, 1-2 μg of luciferase reporter plasmid was cotransfected with 100 ng of pcDNA 3.1/myc-His/lacZ vector and β-galactosidase activity was measured utilizing the β-Galactosidase Enzyme Assay System (Promega) for normalization of results. Background measurements were uniformly low and cell survival was indistinguishable in all transfections performed. The cells were harvested 24 hr later for assay using the Luciferase Assay System (Promega). The luciferase assay was performed using the Luciferase Assay system from Promega following the manufacturer's protocol. Relative luminescence values were measured in a Beckman Scintillation counter with a wide open window. Through this approach, we sought to determine which elements in the 5′ coding sequence acted as transcriptional repressors in a mammalian cell line. Constructs corresponding to each of the first four 123 nucleotide sequences encoding truncated sequences from the 5′ of nucleotide 315 greatly inhibited luciferase expression (on average ˜80%) while segments further 3′ were not inhibitory in comparison with control vector (p<0.001) (FIG. 39).

FIG. 36 Identification of a Regulatory Domain Within the Coding Region of iPLA₂γ Using a Luciferase Reporter Assay System. The bar graph indicates the relative fluorescence value (RFV) of iPLA₂γ-pGL3P constructs compared with unmodified pGL3P vector control used in the luciferase assay system. PCR primer pairs used to prepare each of the 88, 87, 83, 80, and 74 constructs are shown in Table I. Below is a representation of the locations and sizes of iPLA₂γ sequence within each of the constructs in comparison with full-length iPLA₂γ coding sequence (solid bar). The positions of the 88, 77, 74, and 63 kDa start sites is indicated above the bar. A representation of a construct is shown at the bottom with the locations of the SV40 promoter, the iPLA₂γ insert, and luciferase gene indicated.

### EXAMPLE 15

Demonstration of a Nuclear Protein Binding Site within the Transcriptional Regulatory Region of iPLA₂γ. Gel shift analyses were performed with the Promega Gel Shift Assay System according to the manufacturer's specifications by using 2 μg of nuclear protein for each gel shift reaction. For analysis of the 5′ transcription inhibitory region of iPLA₂γ, double-stranded CdXa oligonucleotide containing 5′ iPLA₂γ, sequence and corresponding to a region containing CdXa transcriptional binding sites as predicted by Transfact computer program (5′-TATTAATCTGACTGTAGATATATATATTTACCTCCTTAGTAATGC-3′ (SEQ ID NO: 104) was end-labeled with [−³²P]ATP by using T₄ polynucleotide kinase, as instructed by the manufacturer (Promega). Competition studies were performed by adding a 10-fold molar excess of unlabeled oligonucleotide or randomized control oligonucleotide (5′-TTGATAGTTATCTATTACAGTCTTCTTAGATTGAAACAA-3′) (SEQ ID NO: 94) to the reaction mixture prior to the addition of radiolabeled probe. Reaction mixtures were analyzed on 4% nondenaturing polyacrylamide gels in 0.5 Å˜ TBE (89 mM Tris-HCl, pH 8.0; 89 mM boric acid; 2 mM EDTA) as the running buffer. Electrophoresis was performed at 100 V for 2 to 3 h, followed by drying of the gel at 80° C. under vacuum and visualization of DNA-protein complexes by autoradiography for 12 to 18 h. For analysis of the pre-exon 2 promoter region, Gel shift analysis utilizing radiolabeled CdxA nucleotide (corresponding to residues 6-50 starting from the 88 kDa AUG codon) revealed a single shifted protein-radiolabeled DNA complex utilizing HeLa nuclear extract which was competed out with a 100-fold molar excess of unlabeled CdxA but not with control oligonucelotide (FIG. 37).

FIG. 37 Gel Mobility Shift Analysis of the iPLA₂γ Regulatory Domain. Gel shift utilizing CdxA 1 oligo. Lanes: 1) Negative control minus HeLa nuclear extract, 2) Positive control containing HeLa nuclear extract, 3) Competitive assay containing 10 fold excess CDXA1 oligonucleotide, 4) Noncompetitive assay containing 10-fold excess randomized control oligonucleotide.

### EXAMPLE 16

Tissue Specific Translational Regulation of iPLA₂γ in Myocardium. Due to the obvious complexity of the regulation of iPLA₂γ resulting from the combined presence of transcriptional regulation, complex mRNA processing and utilization of MyoD promotor elements, we recognized that current hypotheses relegating the role of iPLA₂γ exclusively to peroxisomal lipid metabolism were likely limited in appropriate scope. In prior work, we identified robust amounts of calcium independent phospholipase A2 activity in the mitochondrial compartment of both canine and human hearts ( ). Moreover, we recognized early on that the iPLA₂ family of proteins had the potential for providing substrate for mitochondrial fatty acid oxidation by lipid hydrolysis, for regulating complex protein networks by generation of lipid signaling molecules(eicosanoids and lysolipids) and for modulating ion channel structure and function ( ). Accordingly, we considered the possibility that myocardium could process iPLA₂γ in ways completely distinct from that manifest in liver, and that the large amounts of iPLA₂ activity we previously characterized in mammalian mitochondria (including human mitochondria) could result, at least in part, from tissue specific processing of the iPLA₂γ gene. Subcellular fractionation of mouse and rat heart by differential centrifugation was performed essentially as described previously for rat liver (jan). In brief, rat heart was minced and then homogenized in three volumes (w/v) of homogenization buffer (0.25 M sucrose, 5 mM MOPS, pH 7.4, 1 mM EDTA and 0.1% (v/v) of ethanol, 0.2 mM dithiothreitol containing protease inhibitors (phenylmethylsulfonyl fluoride (0.2 mM), leupeptin (1 μg/ml), aprotinin (1 μg/ml) and phosphoramidon (15 μg/ml) using a Potter-Elvehjem homogenizer at 1000 rpm with 8-10 strokes. Homogenate was then centrifuged at 100×g for 10 min to collect cellular debris. Nuclear fraction pelle was obtained following 1000×g for 10 min of the supernatant. Heavy mitochondrial fraction pellet was obtained by centrifugato3000×g for 20 min centrifugation of the light mitochondrial supernatant. Light mitochondrial fraction pellets were obtained by 25,300×g or 70,000×g for 20 min. Finally, 100,000×g was utilized to produce a microsomal pellet and cytosolic fraction. Differential centrifugation demonstrated that iPLA₂γ cosedimented with mitochondria, in particular the light mitochondrial fraction (FIG. 38). Remarkably, the entire series of predicted high molecular weight iPLA₂ proteins were present in rat mitochondria corresponding to utilization of translation initiation sites encoding proteins with Mr=63,74,77, and 88 kDa protein products. These results identified the tissue specific translational regulation of iPLA₂γ in rat myocardium and identified the molecular identity of at least one iPLA₂ activity we previously characterized in mammalian mitochondria.

FIG. 38 Immunoblot Analysis of iPLA₂γ in Subcellular Fractionations of Rat Heart. Equivalent subcellular fractions (100 ug) of rat heart were subjected to SDS PAGE and analyzed by Western blotting. Lanes: 1, rat heart homogenate; 2, crude pellet; 3, heavy mitochondrial fraction; 4, light mitochondrial fraction; 5, A 70,000×g light mitochondrial fraction; 6, Nuclear fraction. Molecular weight markers are indicated on the left and the relative migrations of the 88, 77, 74 and 63 kDa iPLA₂γ (Mancuso et al, J. Biol. Chem. 275:9937 (2000); Tanaka, et al, BBRC 272:320 (2000))polypeptides are indicated by the arrows on the right.

### EXAMPLE 17

Inr sequences typically occur at the transcription start site in genes ( ). An (Inr) sequence with a consensus sequence of Py-Py-A-N-T/A-Py-Py was identified in the pre exon 2 promoter region of iPLA₂γ (FIG. 39). Inr oligonucleotide (5′-GCG TCA CTT CCG CTG GGG GCG G-3′) (SEQ ID NO: 77) was used in competitive competition while randomized control oligo 5′-GTGGCCGGGTGGTCCACCTCGG-3′) (SEQ ID NO: 93) was used for noncompetitive competition in gel shift analysis. Gel shift analysis provided evidence that a specific transcriptional regulatory protein binds to the Inr region in pre-exon 2 sequence (FIG. 40). The second region of 11 nt in length is of unknown function and located near the previously identified start of exon 2 (,). It is interesting to note that this region of high conservation includes a “AG” dinucleotide and thus potentially could identify an intron/exon boundary.

FIG. 39. Alignment of Mouse, Rat, and Human Pre-exon 2 Sequence. Boxed regions indicate highly conserved regions between mouse, rat, and human. A putative Inr site is underlined. Previously reported 5′ ends of the iPLA₂γ (Mancuso et al, J. Biol. Chem. 275:9937 (2000): Tanaka et al, BBRC 272:320 (2000)) message are indicated (<).

FIG. 40. Competitive gel retardation analysis of iPLA₂γ regions. Gel shift utilizing INR oligonucleotide. Lanes: 1) Negative control minus HeLa nuclear extract, 2) Positive control containing HeLa nuclear extract, 3) Competitive assay containing 10 fold excess INR oligonucleotide, 4) Noncompetitive assay containing 10-fold excess randomized control oligonucleotide. Results are representative of three separate Gel shift assays.

### EXAMPLE 18

Identification of MyoD Binding Sites (E-boxes) within the Pre-exon 1 Region of the iPLA₂γ Gene. We noted the presence of sequence corresponding to three E-boxes containing the MyoD consensus binding sequence 5′-CAGGTG-3′)SEQ ID NO: 61) within the 350 nt upstream of exon 1 suggesting that promoter activity may be myoD dependent. To substantiate the functional importance of this assignment, additional experiments were performed. MyoD vector was obtained from Michael Chin (Harvard Medical School) who used it to study MyoD-dependent activation of the myogenin promoter. This vector was used in the Promega luciferase assay system along with iPLA₂γ promoter constructs containing 600nt sequence upstream of exons 1 or 2 inserted upstream via HindIII/NcoI sites into the promoterless vector pGL3E from Promega. Empty pGL3E vector and the SV40 containing promoter vector pGL3P were used as controls. Results were normalized to β-gal resulting from cotransfection with a LacZ vector. Cotransfection of CV1 cells with MyoD and pre-exon 1 containing vectors resulted in a 5 fold stimulation of promoter activity compared with MyoD+pGLE vector cotransfection (FIG. 41). These results suggest the importance of iPLA₂γ in muscle tissues.

FIG. 41. MyoD Stimulation of Promoter Activity in Pre-exon 1 Sequence of iPLA₂γ. CV1 cells were transfected with promoterless vector (pGLE) or with a construct in which pre exon 1 sequence is inserted upstream of the luciferase gene in pGLE (pre-exon 1). Luciferase expression measured as relative luminescence value was determined with (+) or without (−) cotransfection with myoD vector. P<0.01. Sequence of the pre exon 1 promoter region is indicated below. Locations of E-boxes (myoD binding sites) are underlined.

### EXAMPLE 19

Identification of Mitochondrial Import Sequences in iPLA₂γ and Demonstration of Their Functional Integrity. Recent increases in our understanding of mitochondrial import machinery has led to the generation of computer algorithms which can accurately assess the potential for mitochondrial localization of a peptide with great accuracy and predictive probabilities. Using Psort II (http://psort.nibb.acjp/) and MitoProt II 1.0a4 (http://www.mips.biochem.mpg.de/cgi-bin/proj/medgen/mitofilter) online, human iPLA₂γ was predicted to have a high likelihood (>95%) of having an N-terminal mitochondrial localization signal. Since human iPLA₂γ has multiple translation initiation sites resulting in proteins of 88 kDa, 77 kDa, 74 kDa, and 63 kDa, sequences for each of these forms were also utilized in the prediction programs. The identical cleavage site was predicted for the 88 kDa, 77 kDa, and 74 kDa isoforms, while the 63 kDa form was not predicted to have a mitochondrial cleavage site. The 74 kDa mitochondrial localization signal is MISRLAQFKPSSQILRK Δ VS . . . (SEQ ID NO: 58) where the triangle “Δ” indicates the cleavage site between residues lysine (K) and valine (V). To confirm the mitochondrial localization of the iPLA₂γ polypeptide experimentally by yet another independent method, the widely accepted technique of dual image analysis overlay was employed. A commercially available marker for mitochondrial localization (pDsRed2-Mito, BD Biosciences) was employed which encodes mitochondrial targeting sequence of human cytochrome c oxidase fused to red fluorescent protein was utilized as a mitochondrial marker.. To prepare the iPLA_{2γ}-GFP fusion protein construct, site directed mutagenesis was used to make a construct expressing a fusion protein consisting of residues 1-17 of iPLA_{2γ} 74 kDa polypeptide at the N-terminus of GFP. In brief, complementary phosphorylated oligonucleotides were synthesized encoding the predicted 17 amino acid mitochondrial signal of iPLA_{2γ}. The oligonucleotides were also designed so that, upon annealing, they generate appropriate terminal restriction sites for ligation to restriction sites at the 5′ end of the GFP coding sequence in pEGFP vector, thus generating the appropriate fusion construct. Single or cotransfections of Hela cells with these vectors were performed. After 48 h, cells were fixed with 4% paraformaldehyde, coverslipped and fluorescence analyzed by confocal microscopy.

FIG. 42. Import of GFP into mitochondria mediated by an N-terminal iPLA₂γ mitochondrial import signal. HeLa cells were transfected singly or cotransfected with 74GFP and DSRed2-Mito constructs and visualized by confocal microscopy. The co-localization of 74GFP and DsRED2-mito was revealed by the nearly identical staining patterns and by the yellow color resulting from combination of green and red fluorescence.

Dual transfection of HeLa cells with the leader sequence of the 74 kDa polypeptide proximal to GFP (residues 1-32 of the 74 kDa product) and an authentic mitochondrial localization sequence (human cytochrome c oxidase in pDsRed2-Mito) demonstrated that the 74 kDa leader sequence of iPLA₂ resulted in a green punctate pattern and a red punctate protein, respectively (FIG. 42). Critically, merging of the two images yielded an intense yellow punctate pattern indicative of mitochondrial localization mediated by the iPLA₂γ mitochondrial leader sequence (FIG. 42). Thus, by three independent techniques, buoyant density gradient centrifugation, computer prediction of mitochondrial localization sequence(localization in silico), and colocalization of the iPLA₂γ mitochondrial leader sequence-tagged green fluorescent protein with an authentic mitochondrial marker, the results demonstrate that iPLA₂γ is localized, at least in part, to the mitochondrial compartment in myocardium in contrast to its predominant peroxisomal localization in liver. Moreover, these results demonstrate dual localization sequences in a single protein and thus identify a competition for newly synthesized iPLA₂γ between mitochondrial localization(from its N terminal mitochondrial localization sequence) and peroxisomal localization (FIG. 42) (from its C terminal SKL peroxisomal localization sequence).

All references cited in this specification are hereby incorporated by reference. The discussion of the references herein is intended to summarize the assertions made by their authors and no admission is made as to the accuracy or pertinency of the cited references or that any reference is material to patentability.

While the invention has been described in terms of various specific embodiments in the examples, those skilled in the art will recognize that the invention can be practiced with modification within the spirit and scope of the claims which follow.

1. An isolated nucleic acid molecule comprising a polynucleotide encoding a phospholipase A2_{γ} polypeptide. (SEQ ID NO:1)

2. An isolated nucleic acid molecule in accordance with claim 1, wherein said phospholipase A2γ polypeptide catalyzes cleavage of fatty acids from the sn-2-position of phospholipids.

3. An isolated nucleic acid molecule in accordance with claim 2 wherein said polynucleotide encodes a sequence as set forth in SEQ ID NO: 1 or SEQ ID NO:2.

4. A vector comprising a nucleic acid molecule in accordance with claim 1.

5. A cell transformed or transfected with a vector in accordance with claim 4.

6. An isolated nucleic acid molecule comprising a fragment of a polynucleotide encoding a phospholipase A2γ wherein said fragment specifically hybridizes with a sequence as set forth in at least one of SEQ ID NOS 3, SEQ ID NO:4 and SEQ ID NO: 5.

7. An isolated nucleic acid comprising a polynucleotide having at least about 90% identity with SEQ ID NO:3, SEQ ID NO:4, SEQ ID NO:5, or SEQ ID NOS 6, 7, 8 or 9 wherein the encoded polypeptide has or modulates enzymatic activity.

8. An isolated nucleic acid according to claim 7 comprising SEQ ID NO:3, SEQ ID NO:4, SEQ ID NO:5, or SEQ ID NOS 6, 7, 8 or 9.

9. An antisense sequence which specifically hybridizes to SEQ ID NO: 3, or SEQ ID NO:4, SEQ ID NO: 5 or SEQ ID NO: 6.

10. An isolated polypeptide comprising a phospholipase A2γ.

11. An isolated polypeptide in accordance with claim 10 which catalyzes cleavage of fatty acids from the sn-2-position of phospholipids.

12. An isolated polypeptide in accordance with claim 11 which has at least 90% identity with SEQ ID NO: 1 or SEQ ID NO:2.

13. An isolated polypeptide in accordance with claim 12 comprising SEQ ID NO:1 or SEQ ID NO:2.

14. An isolated polypeptide in accordance with claim 12 which is a conservatively substituted variant of SEQ ID NO:1 or SEQ ID NO:2.

15. An antibody capable of binding to a phospholipase A₂γ according to claim 1.

16. A vector comprising a nucleic acid molecule in accordance with claim 1 suitable for vectoring into a transgenic mouse wherein the reporter gene encodes an enzyme capable of being detected by a calorimetric, fluorometric or luminometric assay.

17. A method in accordance with claim 16 wherein said reporter gene encodes a luciferase.

18. A method in accordance with claim 30 comprising administering to the mouse an iPLA₂γ polypeptide as set forth in SEQ ID NO: 1, SEQ ID NO:2 or a conservatively substituted variant thereof or administering a polynucleotide encoding said iPLA_{2γ} polypeptide wherein the repressor binding site comprises SEQ ID NO:10.

19. A genetically engineered cell in accordance with claim 18 wherein said reporter gene encodes and enzyme capable of being detected by a calorimetric, fluorometric or luminometric assay.

20. A genetically engineered cell in accordance with claim 19 wherein said reporter gene encodes a luciferase.

21. A method for preparing a transgenic mouse which further comprises breeding a transgenic founder mouse having SEQ ID 1 stably integrated in its genome with WT B6CBAE1/J mice.

22. A transgenic mouse having in its genome a nucleic acid molecule comprising a polynucleotide encoding a phospholipase A₂γypolypeptide. (SEQ ID NO: 1)

23. A transgenic mouse in accordance with claim 22 wherein said phospholipase A_{2γ} polypeptide catalyzes cleavage of fatty acids from the sn-2-position of phospholipids.

24. A transgenic mouse in accordance with claim 23 wherein said polynucleotide encodes a sequence as set forth in SEQ ID NO:1 or SEQ ID NO:2.

25. A vector which can be used to generate a transgenic mouse in accordance with claim 24.

26. A transgenic mouse having within its genome a nucleic acid molecule comprising a fragment of a polynucleotide encoding a phospholipase A₂γ wherein said fragment specifically hybridizes with a sequence as set forth in SEQ ID NOS:3 or SEQ ID NO:4 or SEQ ID NO:5.

27. A transgenic mouse in accordance with claim 6 wherein said fragment comprises a polynucleotide having at least about 90% identity with SEQ ID NO:3, SEQ ID NO:4, SEQ ID NO:5, or SEQ ID NOS 6, 7, 8 or 9 wherein the encoded polypeptide has or modulates enzymatic activity.

28. A transgenic mouse having within its genome a nucleic acid according to claim 7 comprising SEQ ID NO:3, SEQ ID NO:4, SEQ ID NO:5, or SEQ ID NOS:6, 7, 8 or 9.

29. A transgenic mouse in accordance with claim 8 having within its genome a nucleic acid having an antisense sequence which specifically hybridizes to SEQ ID NO:3, or SEQ ID NO:4, SEQ ID NO:5 or SEQ ID NO:6.

30. A genetically engineered cell in accordance with claim 31 wherein said repressor binding site comprises SEQ ID NO: 10.

31. A mitochondrial targeting sequence MISRLAQFKPSSQILRKΔVS (SEQ ID NO:58) at the N-terminus of the 74 kDa iPLA₂γ product.

32. A mitochondrial import signal and cleavage site (LRK/VS) (SEQ ID NO:95) immediately downstream from the 74 kDa alternative start site which directs iPLA₂γ into mitochondria resulting in a truncated protein of approximately 72 kDa.

33. A subcellular localization of iPLA₂γ into both peroxisomes and mitochondria which may have important implications for the role of iPLA₂γ in modulating cellular function.

34. An alternative exon 5 splice variant utilizing gt/ag splice junction and resulting in a novel 5 amino acid change (ASCSV) SEQ ID NO:28.

35. iPLA₂γ exons designated exons 1 (SEQ ID NO:29) and 4 (SEQ ID NO:30) corresponding to genomic sequence 135327-135622 and 125460-125571 of GenBank genomic clone RG054D04.

36. A truncated iPLA_{2γ} 63 kDa (SEQ ID NO: 21) resulting from initiation at methionine number 122 of iPLA₂γ which is expressed in the baculoviral and in vitro expression systems at least 20 fold greater (at the mRNA and protein levels) than the full-length 88 kDa (SEQ ID NO: 1) protein product.

37. A transgenic construct containing the γ MHC promoter upstream of the full-length iPLA₂γ coding sequence (SEQ ID NO: 6) for myocardial specific expression of recombinant iPLA₂γ in TGiPLA₂γ mice.

38. A transgenic mouse (TGiPLA₂γ)which expresses 77 kDa, 74 kDa, 63 kDa, and 45 kDa isoforms of recombinant human iPLA₂γ.

39. A polypeptide (SEQ ID NO: 1) with alternative ATG start sites encoding 88, 77, 74, and 63 kDa iPLA₂γ proteins

40. An in vitro expression construct in which truncated iPLA₂γ sequences (SEQ ID NO: 6, 15, 18, and 21) are cloned downstream from the SV40 promoter of vector pEF.

41. A transcription factor binding region defined by gel shift analysis between nucleotide residues 6-50 encoding the 88 kDa protein and including the sequence 5′-TATTAATCTGACTGTAGATATATATATTTACCTCCTTAGTAATGC-3′ (SEQ ID NO:59) within the N-terminal coding region of iPLA₂γ.

42. Three MyoD transcription factor binding sites (E-boxes) defined by the consensus nucleotide sequence CANNTG in promoter 1 (pre exon 1) sequence of the iPLA_{2γ} gene corresponding to nucleotide residues −22 thought −27 corresponding to nucleotide sequence 5′-CAAGTG-3′ (SEQ ID NO: 60), −53 through −58 corresponding to nucleotide sequence 5′-CAGGTG-3′ (SEQ ID NO:61), and −349 through −354 corresponding to nucleotide sequence 5′-CAGGTG-3′ (SEQ ID NO:62) upstream from start of exon 1.

43. An initiator (Inr) sequence with a consensus sequence of Py-Py-A-N-T/A-Py-Py at which nuclear protein constituents bind to 5′-GCG TCA CTT CCG CTG GGG GCG G-3′ (SEQ ID NO: 77) at nucleotide residues −54 through −75 upstream from the putative start of exon 2.

44. A pre exon 2 sequence 5′-GCCAGTGTTTG-3′ (SEQ ID NO: 78) which is consistent with a CORE promoter element was identified in comparisons of human, mouse, and rat sequence.

45. A transcriptional regulatory domain within the 5′ coding region (nucleotide residues 1-315)(SEQ ID NO: 57) of iPLA₂γ.

46. A nuclear binding domain corresponding to SEQ ID NO:59 defined by gel shift analysis within the transcriptional regulatory domain.

47. iPLA₂γ exons SEQ ID NO:29 and 30 corresponding respectively to exons 1 and 4.

48. A novel splice variant X resulting from splicing exon 1 and truncated exon 5 sequence (SEQ ID NO:5).
